(19) United States
(12) Patent Application Publication
(10) Pub. No.: US 2021/0277052 A1
(43) Pub. Date: Sep. 9, 2021

(54) RECOMBINANT GRP94 CHAPERONES AND METHODS OF USING THE SAME TO MODULATE GLYCOSYLATION IN FUSION PROTEINS

(71) Applicant: Hauptman-Woodward Medical Research Institute, Buffalo, NY (US)

(72) Inventors: Daniel T. Gewirth, Buffalo, NY (US); John D. Huck, New Haven, CT (US)

(21) Appl. No.: 17/191,387

(22) Filed: Mar. 3, 2021

Related U.S. Application Data
(60) Provisional application No. 62/984,545, filed on Mar. 3, 2020.

(51) Int. Cl. C07K 1/107; C12N 9/10; C07K 14/47

(52) U.S. Cl. CPC C07K 1/1077; C12N 9/1051; C07K 2319/21; C07K 2319/91; C07K 2319/43; C07K 14/47

(57) ABSTRACT

Methods for glycosylation of Grp94 chaperone proteins, including Grp94 fragments, mutants, and fusion proteins, expressed in mammalian cells are presented. The method comprises altering a Pre-N domain of the Grp94 chaperone protein, wherein the alteration of the Pre-N domain of the Grp94 chaperone protein leads to glycosylation at available glycosylation sites downstream of the Pre-N domain of the Grp94 chaperone protein, when expressed in mammalian cells.

### CROSS-REFERENCE TO RELATED APPLICATIONS

This application is a utility application filed under applicable portions of 35 U.S.C. § 111 and 37 CFR § 1.53, which claims priority to U.S. Provisional Application No. 62/984,545, filed on Mar. 3, 2020. The entire contents of the foregoing application is expressly incorporated herein by reference.

### SEQUENCE LISTING

The instant application contains a Sequence Listing, which has been submitted electronically in ASCII format and is hereby incorporated by reference in its entirety. Said ASCII copy, created on Mar. 1, 2021, is named SequenceListing3102632US02 ST25.txt and is 321,853 bytes in size.

### TECHNICAL FIELD

This application is generally directed to the field of molecular biology and more specifically to methodology for using the Grp94 Pre-N Domain to regulate N-linked glycosylation in order to preserve chaperone function.

### BACKGROUND

U.S. Pat. No. 7,589,174 to Argon et al., titled “GRP94-BASED COMPOSITIONS AND METHODS OF USE THEREOF,” filed May 12, 2004, is directed to mini chaperones and methods of use thereof for the treatment of cancer and other disorders. Also disclosed are tools to facilitate screening therapeutic agents that have selective binding affinity for GRP94. However, U.S. Pat. No. 7,589,174 is silent regarding the role of the Pre-N domain in glycosylation of minor glycosylation sites in Grp94.

U.S. Pat. No. 8,404,805 to Nicchitta et al., titled “CHARACTERIZATION OF GRP94-LIGAND INTERACTIONS AND PURIFICATION, SCREENING, AND THERAPEUTIC METHODS RELATING THERETO,” filed Oct. 5, 2009 is directed to characterization of interactions between ligands and Hsp90 proteins, including GRP94, wherein ligand binding to the N-terminal nucleotide binding domain of GRP94 elicits a conformational change that converts the GRP94 from an inactive to an active conformation, and wherein the chaperone and peptide-binding activities of the GRP94 are markedly stimulated. Also disclosed are purification, screening, and therapeutic methods pertaining to the biological activity of GRP94, and in some instances HSP90, based upon the characterization of ligand interactions of Hsp90 peptide-binding proteins, including GRP94. However, U.S. Pat. No. 8,404,805 fails to appreciate that the Pre-N domain regulates glycosylation of minor glycosylation sites in Grp94.

Accordingly, there remains a need in the art to control glycosylation of proteins for use as therapeutics, in screening assays, as research tools, and in the production of antibodies.

### BRIEF SUMMARY

A challenge in the field of recombinant protein production is achieving substantially the same glycosylation pattern as the native protein, so that the recombinant protein exhibits substantially the same structure, function, activity, etc. The ability to control glycosylation of biologics, drug targets, antigens, and other proteins of interest would constitute an advancement in the art. Therefore, the inventive subject matter includes recombinant Grp94 proteins, mutants, and fragments thereof capable of regulating glycosylation of glycosylation sites downstream of the Grp94 Pre-N domain. The inventive subject matter also includes fusion proteins comprising the recombinant Grp94, mutants, and fragments thereof, therapeutic proteins, drug screening targets, antibodies, and other proteins of interest and methods of making and using the same.

In one embodiment, a method for glycosylation of Grp94 chaperone proteins expressed in mammalian cells is presented. For instance, the method comprises altering a Pre-N domain of the Grp94 chaperone protein, wherein the alteration of the Pre-N domain of the Grp94 chaperone protein leads to glycosylation at available glycosylation sites downstream of the Pre-N domain of the Grp94 chaperone protein, when expressed in mammalian cells.

In another embodiment, presented herein is a method for full glycosylation of any protein expressed as a fusion protein at a C-terminus of a Grp94 chaperone protein when the protein is expressed in mammalian cells. The method comprises altering a Pre-N domain of the Grp94 chaperone protein.

In a further embodiment, presented herein is a method for full glycosylation of any protein expressed as a fusion protein at a C-terminus of a Pre-N domain of a Grp94 chaperone protein, when the protein is expressed in mammalian cells. The method comprises altering a Pre-N domain of the Grp94 chaperone protein.

The alterations may include one or more of the following: deleting the Pre-N domain of the Grp94 chaperone protein; deleting residues 22-47 of the Pre-N domain; deleting one or more amino acids of the Pre-N domain of the Grp94 chaperone protein; substituting residues of the Pre-N domain with a different amino acid; or inserting one or more amino acids into the Pre-N domain, at any position.

In one embodiment, a recombinant fusion protein includes Grp94 and a glycosylated protein fused to the C-terminus of Grp94. The Grp94 may be wild type Grp94 or a recombinant Grp94. The glycosylated protein is glycosylated at one or more glycosylation sites as a result of being expressed downstream from the Grp94. Exemplary glycosylated proteins may be glycosylated at one, two, three, four, five or more glycosylation sites following expression of the recombinant fusion protein in a host cell. In some embodiments, the glycosylated protein in the recombinant fusion protein is glycosylated at all potential glycosylation sites, i.e., fully glycosylated.

In an embodiment that includes the recombinant Grp94, suitable recombinant Grp94 include substitution of five or more residues or deletion of seven or more residues from a Pre-N domain of the recombinant Grp94. For example, the recombinant Grp94 may include deletion of residues 22 to 32 from the Pre-N domain (e.g., SEQ ID NO. 2), deletion of residues 22 to 47 from the Pre-N domain (e.g., SEQ ID NO. 3, or deletion of the Pre-N domain (e.g., SEQ ID NO. 4).

The recombinant Grp94 may also include substitution of five or more residues of the Pre-N domain with alanine. Residues 33 to 37 of the Pre-N domain may be substituted with alanine (e.g., SEQ ID NO. 5). Residues 38 to 42 of the Pre-N domain may be substituted with alanine (e.g., SEQ ID NO. 6). Residues 33 to 37 of the Pre-N domain may be substituted with alanine (e.g., SEQ ID NO. 7). Residues 48 to 52 of the Pre-N domain may be substituted with alanine (e.g., SEQ ID NO. 8).

A further embodiment includes methods of producing any of the recombinant fusion proteins described herein. The method includes a step of culturing a host cell including a vector, which comprises a nucleic acid encoding the recombinant fusion protein under conditions permitting the production of the recombinant fusion protein. Exemplary vectors include (i) SEQ ID NO. 35, SEQ ID NO. 36, or SEQ ID NO. 34, which encode Grp94 and in which several residues are deleted from the Pre-N domain, and (ii) the nucleic acid sequence encoding the protein fused to the Grp94. Suitable vectors also include (i) SEQ ID NO. 48, SEQ ID NO. 49, SEQ ID NO. 50, or SEQ ID NO. 51, which encode Grp94 and in which several residues are substituted with alanine, and (ii) the nucleic acid sequence encoding the protein fused to the Grp94. The recombinant fusion protein may also be recovered.

In another embodiment, a method of glycosylating a protein includes the steps of expressing any of the recombinant fusion proteins described herein in a host cell. When the recombinant fusion protein includes a cleavable linker, the method may include a step of cleaving the protein from the wild type or recombinant Grp94. The recombinant fusion protein may also include a tag, such as a histidine tag, a flag tag, or a biotin tag that facilitates isolation of the recombinant fusion protein or the tagged portion thereof. In some embodiment(s), the protein is glycosylated at a glycosylation sequon, such as Asn-X-Ser or Asn-X-Thr (X is any amino acid except proline). Suitable host cells may be mammalian, including human embryonic kidney line (e.g., HEK293).

In further embodiments, the glycosylated protein produced by the methods described herein is a drug target and may be used as substrates for drug screens. In another embodiment, the glycosylated protein is a therapeutic protein that may be formulated into a pharmaceutical composition and optionally included in a kit with reagents, supplies, and/or instructions for use. In another embodiment, the glycosylated protein is an antigen that can be used to produce antibodies for use in assays, protein purification, or as therapeutic antibodies.

The above embodiments are intended to be exemplary only, wherein other suitable embodiments will be apparent to those of sufficient skill. These other embodiments are within the scope of the disclosed subject matter. Furthermore, a number of references are cited throughout this application, each of which are herein incorporated by reference in their entirety.

### BRIEF DESCRIPTION OF THE DRAWINGS

So that the manner in which the features of the disclosure can be understood, a detailed description may be had by reference to certain embodiments, some of which are illustrated in the accompanying drawings. It is to be noted, however, that the drawings illustrate only certain embodiments and are therefore not to be considered limiting of its scope, for the scope of the disclosed subject matter encompasses other embodiments as well. The drawings are not necessarily to scale, emphasis generally being placed upon illustrating the features of certain embodiments. In the drawings, like numerals are used to indicate like parts throughout the various views.

FIGS. 1A-1D show that truncation of the Pre-N domain leads to excessive N-linked glycosylation.

FIG. 2A-2F show that mutation of minor N-linked glycosylation sites restores chaperone function to Pre-N truncated constructs.

FIG. 3 shows glycosylation Modeling at Asn107 of the Grp94 NTD.

FIGS. 4A-4C show Flow Cytometry Experiments and the results of an ATP hydrolysis assay.

FIG. 5 shows alignment of Pre-N Domain Sequences Across Metazoans.

FIG. 6A-6B show that the Function of the Pre-N domain is highly sequence specific.

FIGS. 7A-7C show that HgGrp94 forms heterogeneous oligomers.

FIGS. 8A-8B show structural inspection of Grp94 Glycosylation Sites.

Corresponding reference characters indicate corresponding parts throughout several views. The examples set out herein illustrate several embodiments, but should not be construed as limiting in scope in any manner.

### DETAILED DESCRIPTION

The present disclosure relates to using the Grp94 Pre-N Domain to regulate N-linked glycosylation in order to preserve chaperone function. Many proteins in mammalian cells that are expressed in the endoplasmic reticulum are co- or post-translationally modified by N-linked glycosylation at glycosylation sequons (N-X-S/T, where X is any amino acid but proline) in the amino acid sequence of the protein.

Protein sequences may contain many N-linked glycosylation sequons, but these sites may not always be modified when the protein is expressed in mammalian cells. Many sites are silent or are used only under certain conditions.

Advantageously, the present technique may be used for scientific, diagnostic, or therapeutic applications to produce a protein in mammalian cells that is fully glycosylated at one or more of the glycosylation sites. The present method directs the cellular machinery to attach glycosylation groups at all available sequons, whether these sequons occur naturally or are introduced into the DNA coding for the protein by mutagenesis.

Grp94 is a highly abundant endoplasmic reticulum (ER) lumenal chaperone that is essential for the maturation of a restricted, yet important subset of proteins in the secretory pathway. The mechanisms by which Grp94 is regulated in the rapidly fluctuating folding environment of the ER remain largely unexplored. Here, the inventors report that N-linked glycosylation inactivates client maturation function. Applicant has found that this process is intrinsically regulated by the Pre-N domain, which serves a protective role by inhibiting glycosylation at functionally deleterious sites. When the Pre-N domain is truncated or mutated, normal mono-glycosylation is fundamentally disrupted, leading to hyper-glycosylation and loss-of-function. Applicant has further found that hyper-glycosylation alters the biochemical characteristics and ATPase activity of Grp94 depending on the availability of certain sites, suggesting that differential glycosylation may tune Grp94 chaperone activity and promote new biological functions. In sum, these findings suggest a molecular mechanism by which the Pre-N domain controls N-linked glycosylation and provide new insight into the regulatory function of this modification for Grp94.

Most newly synthesized proteins destined for the cell surface or secretion must transit through the endoplasmic reticulum (ER) for folding and post-translational processing (Schwarz and Blower, 2016). Grp94 is the ER resident member of the hsp90 family of chaperones and it is specifically required for the maturation of various membrane and secreted proteins, such as GARP, pro-insulin, insulin-like growth factors (IGFs), platelet glycoprotein Ib-IX-V complex, and most members of the LDLR, toll-like receptor (TLR), and integrin families (Zhang et al., 2015) (Ansa-Addo et al., 2016) (Ghiasi et al., 2019) (Randow and Seed, 2001) (Weekes et al., 2012) (Yang et al., 2007). Grp94's control over the fate of these diverse proteins makes it an emerging therapeutic target for number of diseases including, but not limited to type 2 diabetes, familial hypercholesterolemia, and sepsis (Ghiasi et al., 2019) (Poirier et al., 2015) (Li et al., 2019), as well as cancers such as HER2-positive breast cancer and multiple myeloma (Patel et al., 2013) (Liu et al., 2013) (Ansa-Addo et al., 2016). A recent proteomics study identified a much larger set of Grp94-associated proteins (Hong et al., 2017), deepening Applicant's understanding of the cellular importance of this paralog.

Like its hsp90 family members, Grp94 is a homodimeric ATP-driven machine containing three (3) major domains: the N-terminal (NTD), Middle (MD), and C-terminal (CTD) domains (Dollins et al., 2007a; Huck et al., 2017a). The NTD is the site of ATP binding, the MD is involved in ATP hydrolysis, and the CTD is the site of the high affinity homo-dimerization interface (Prodromou, 2016). In addition to the three core domains, hsp90s possess divergent regions of variable length, including a Pre-N domain (Pre-N) that precedes the NTD, a Charged Linker (CL) domain between the NTD and MD, and a C-terminal extension (CX) from the CTD (Maharaj et al., 2016). The hsp90 chaperone cycle involves a series of large conformational shifts between open and closed dimer states that are driven by the binding of ATP (Krukenberg et al., 2011). Recent structural and biophysical studies have begun to clarify how Hsp90 engages and matures its clients (Kirschke et al., 2014) (Karagoz et al., 2014) (Verba et al., 2016) (Dahiya et al., 2019) (Boysen et al., 2019). However, the mechanisms by which Grp94 utilizes ATP to activate its own diverse group of clients remain poorly understood.

ER proteostasis is achieved by matching chaperone availability with protein folding load (Gardner et al., 2013). Despite being required for only a limited subset of the proteins that transit the ER, Grp94 is the most abundant ER protein (Itzhak et al., 2016). This apparent contradiction raises the question as to how Grp94 activity is regulated within a dynamic folding environment to ensure efficient folding under non-stress conditions (Braakman and Hebert, 2013) (Schwarz and Blower, 2016). In addition, ER stress conditions, such as the unfolded protein response (UPR), oxidative imbalance, glucose deprivation, calcium perturbation, hypoxia, and glycosylation blockade, as well as certain cancers further induce expression of Grp94 and other ER chaperones in order to meet the increased folding demands and assist in proteasomal degradation of misfolded proteins (Zheng et al., 2008). BiP, another highly abundant ER chaperone, is the central mediator of protein folding and stress response in the ER. It is now appreciated that BiP possesses a multitude of regulatory mechanisms such as oligomerization, AMPylation, and co-factor modulation to regulate activity in response to changes in client protein load (Preissler et al., 2015a) (Preissler et al., 2015b) (Marcinowski et al., 2011). Likewise in the cytosol, Hsp90 possesses several layers of regulation including various post-translational modifications and dedicated co-chaperones that finely tune ATP hydrolysis activity and client recruitment (Schopf et al., 2017).

In contrast, the regulatory roles of protein modifications and co-chaperones for Grp94 function have long been obscure. N-linked glycosylation is an essential co-translational and, in some cases, post-translational modification of proteins that transit the ER due to its critical roles in protein folding, secretion, and protein-protein interactions (Helenius and Aebi, 2004). N-linked glycosylation is carried out by the oligosaccharyltransferase (OST) complex on asparagine (Asn) residues in the consensus sequon Asn-X-Ser/Thr, where X can be any amino acid except proline (Schwarz and Aebi, 2011). Here, “sequon” refers to the N-linked glycosylation motif (Asn-X-Ser/Thr), whereas “site” specifically refers to the modified Asn residue. Grp94 contains six sequons with acceptor sites at Asn62 in the Pre-N domain, Asn107 and Asn217 in the NTD, and Asn445, Asn481, and Asn502 in the Middle domain (MD). Asn217 is the major site of glycosylation and is nearly constitutively modified under normal conditions (Qu et al., 1994; Wearsch and Nicchitta, 1996). The remaining glycosylation sites have been collectively referred to as minor, cryptic, or silent, to reflect their atypical usage (Dersh et al., 2014b)(Cherepanova et al., 2019). Multi-glycosylated forms of Grp94 have been observed (Qu et al., 1994) (Feldweg and Srivastava, 1995) (Cala, 2000) (Yang et al., 2007) (Dersh et al., 2014b) (Seidler et al., 2014), but the biological importance of these species is almost completely unknown. Furthermore, unlike its cytosolic counterpart, Grp94 does not possess a network of co-chaperones to regulate ATP hydrolysis and client binding. Paralogs of cytosolic co-chaperones do not exist in the ER and the only bona fide co-chaperones identified to date include CNPY proteins, which appear to be client specific (Liu et al., 2010) (Rosenbaum et al., 2014).

It has been recently discovered that the Pre-N domain is required for the maturation and cell surface localization of Grp94 client integrins and TLRs (Huck et al., 2017b). The Pre-N domain is a conserved extension preceding the Grp94 NTD, yet it varies substantially in length and sequence among hsp90s, with the Grp94 Pre-N domain (52 amino acids) dwarfing the Pre-N regions of the other paralogs (Trap 1: 24 aa; Hsp90a: 16 aa; Hsp90(3: 11 aa). Given that the Pre-N domain is both intrinsically suppressive of ATP hydrolysis and that it is essential for client maturation, the inventors postulated previously that this unique region might replace some of the mechanistic functions of a regulatory co-chaperone in the ER by, for example, recognizing and recruiting clients or stabilizing Grp94:client complexes. Here, the inventors set out to uncover the essential role of the Pre-N domain in Grp94 chaperone function. Surprisingly and unexpectedly, the inventors found that the Pre-N domain controls the N-linked glycosylation status of Grp94. Truncations and mutations of the Pre-N domain lead to high occupancy glycosylation that render the chaperone non-functional; an effect that can be reversed by mutating deleterious glycosylation sites to prevent their modification. Applicant has further found that hyper-glycosylation alters the biochemical properties and the ATPase activity of Grp94 depending on the availability of certain sites, suggesting that differential N-linked glycosylation may serve to tune Grp94 activity in the cell and promote new biological functions. Taken together, Applicant's results may explain the evolutionary importance of the Pre-N domain and provide clues into the regulatory roles of N-linked glycosylation for Grp94.

In one embodiment, a recombinant Grp94 protein is derived from human Grp94, monkey Grp94, or canine Grp94. In another embodiment, a recombinant fusion protein includes Grp94 and a glycosylated protein fused to the C-terminus of Grp94. The Grp94 in the fusion protein be wild type Grp94 or a recombinant Grp94 as described herein. Non-limiting examples of Grp94 chaperones that may be employed in the inventive compositions and methods include those listed in Table 1 (SEQ ID NOS. 1-32), encoded by the corresponding DNA sequences (SEQ ID NOS. 33-64). Protein and DNA sequences having about 95%, about 96%, about 97%, about 98%, or about 99% sequence homology are also contemplated.

TABLE 1
Exemplary Protein and DNA Sequences.

| SEQ ID NO. | Protein Sequence | SEQ ID NO. | DNA Sequence |
|---|---|---|---|
| 1. | Grp94 WT | 33. | Grp94 WT |
| 2. | Grp94 D22-32 | 34. | Grp94 DPreN |
| 3. | Grp94 D22-47 | 35. | Grp94 D22-32 |
| 4. | Grp94 DPreN | 36. | Grp94 D22-47 |
| 5. | Grp94 33-37Ala | 37. | Grp94 DPreN, MD3xA |
| 6. | Grp94 38-42Ala | 38. | Grp94 DpreN, MD3xA, S109A |
| 7. | Grp94 43-47Ala | 39. | Grp94 DpreN, S109A |
| 8. | Grp94 48-52Ala | 40. | Grp94 DpreN, N445A |
| 9. | Grp94 DPreN, MD3xA | 41. | Grp94 DpreN, N481A |
| 10. | Grp94 DpreN, MD3xA, S109A | 42. | Grp94 DpreN, N502A |
| 11. | Grp94 DpreN, S109A | 43. | Grp94 DpreN, S109A, N445A. |
| 12. | Grp94 DpreN, N445A | 44. | Grp94 DpreN, S109A, N445A, |
| | | | N481A |
| 13. | Grp94 DpreN, N481A | 45. | Grp94 DpreN, S109A, N445A, |
| | | | N502A |
| 14. | Grp94 DpreN, N502A | 46. | Grp94 D22-32, N62A, S109A, |
| | | | N445A, N481A, N502A |
| 15. | Grp94 DpreN, S109A, N445A. | 47. | Grp94 28-32Ala |
| 16. | Grp94 DpreN, S109A, N445A, | 48. | Grp94 33-37Ala |
| | N481A | | |
| 17. | Grp94 DpreN, S109A, N445A, | 49. | Grp94 38-42Ala |
| | N502A | | |
| 18. | Grp94 D22-32, N62A, S109A, | 50. | Grp94 43-47Ala |
| | N445A, N481A, N502A | | |
| 19. | Grp94 28-32Ala | 51. | Grp94 48-52Ala |
| 20. | Grp94 53, 57Ala | 52. | Grp94 53, 57Ala |
| 21. | Grp94 58-62Ala | 53. | Grp94 58-62Ala |
| 22. | Grp94 63-67Ala | 54. | Grp94 63-67Ala |
| 23. | Grp94 68-72Ala | 55. | Grp94 68-72Ala |
| 24. | Grp94 73-77Ala | 56. | Grp94 73-77Ala |
| 25. | Grp94 78-82Ala | 57. | Grp94 78-82Ala |
| 26. | Grp94 D22-32, S109A | 58. | Grp94 D22-32, S109A |
| 27. | Grp94 D22-32, S109A, N445A | 59. | Grp94 D22-32, S109A, N445A |
| 28. | Grp94 D22-32, S109A, N481A | 60. | Grp94 D22-32, S109A, N481A |
| 29. | Grp94 D22-32, S109A, N502A | 61. | Grp94 D22-32, S109A, N502A |
| 30. | Grp94 D22-32, N481A | 62. | Grp94 D22-32, N481A |
| 31. | Grp94 D22-32, N502A | 63. | Grp94 D22-32, N502A |
| 32. | Grp94 human | 64. | Grp94 human |

In an embodiment that includes the recombinant Grp94, suitable recombinant Grp94 include substitution of five or more residues or deletion of seven or more residues from a Pre-N domain of the recombinant Grp94. In some embodiments, recombinant Grp94 includes deletion of 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 amino acid residues from the Pre-N region. In some embodiments, recombinant Grp94 may include deletion of about 13% to 15%, about 15% to about 25%, about 25% to about 50%, about 50% to about 70%, about 70% to about 90%, or about 90% to about 95% of the residues from the Pre-N domain. It should be appreciated that such deletions may be random or sequential. Deletions may begin with amino acid residue 22, 23, 24, 25, 26, 27, 28, 29, 30, 31, 32, or any amino acid residue downstream therefrom, provided there are sufficient amino acid residues to be deleted from the Pre-N domain. For example, the recombinant Grp94 may include deletion of residues 22 to 32 from the Pre-N domain (e.g., SEQ ID NO. 2), deletion of residues 22 to 47 from the Pre-N domain (e.g., SEQ ID NO. 3), or deletion of the Pre-N domain (e.g., SEQ ID NO. 4). In some embodiments, the recombinant Grp94 has about 95%, 96%, 97%, 98%, or 99% sequence homology with SEQ ID NO. 2, SEQ ID NO. 3, or SEQ ID NO. 4.

The recombinant Grp94 may also include substitution of five or more residues of the Pre-N domain. In some embodiments, recombinant Grp94 may include substitution of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 amino acid residues in the Pre-N region. In some embodiments, recombinant Grp94 may include substitution of about 13% to 15%, about 15% to about 25%, about 25% to about 50%, about 50% to about 70%, about 70% to about 90%, or about 90% to about 95% of the residues from the Pre-N domain. It should be appreciated that such substitutions may be random or sequential. Deletions may begin with amino acid residue 22, 23, 24, 25, 26, 27, 28, 29, 30, 31, 32, or any amino acid residue downstream therefrom, provided there are sufficient amino acid residues to be substituted from the Pre-N domain. In one embodiment, amino acid residues in the Pre-N region of Grp94 are substituted with alanine. In another embodiment, amino acid residues in the Pre-N region of Grp94 are substituted with alanine, glycine, valine, leucine, and/or isoleucine. Residues 33 to 37 of the Pre-N domain may be substituted with alanine (e.g., SEQ ID NO. 5). Residues 38 to 42 of the Pre-N domain may be substituted with alanine (e.g., SEQ ID NO. 6). Residues 33 to 37 of the Pre-N domain may be substituted with alanine (e.g., SEQ ID NO. 7). Residues 48 to 52 of the Pre-N domain may be substituted with alanine (e.g., SEQ ID NO. 8).). In some embodiments, the recombinant Grp94 has about 95%, 96%, 97%, 98%, or 99% sequence homology with SEQ ID NO. 5, SEQ ID NO. 6, SEQ ID NO. 7, or SEQ ID NO. 8. In some embodiments, the Pre-N domain of recombinant Grp94 includes a combination of deletion and substitution of residues.

Suitable glycosylated proteins (also referred to as glycoproteins) are listed in Table 2. In one embodiment, the glycosylated protein comprises a fragment of one of the glycoproteins listed in Table 2. In another embodiment, the glycosylated protein comprises a mutant of one of the glycoproteins listed in Table 2. In a further embodiment, the glycosylated protein is a fusion protein comprising a fragment of one of the glycoproteins listed in Table 2 and another protein. The glycosylated protein may be fused to Grp94 directly or indirectly via a linker. Suitable linkers include Tobacco Etch Virus protease cleavage sites (Glu-Asn-Leu-Tyr-Phe-Gln-(Gly/Ser)) and those described by Xiaoying Chen et al. in Design of an in vivo cleavable disulfide linker in recombinant fusion proteins, 49 BIOTECHNIQUES 513-18 (July 2010) and Xiaoying Chen et al. in Fusion protein linkers: Property, design, and functionality, 65(10) ADVANCED DRUG DELIVERY REVIEWS 1357-69 (October 2013), the contents of which are incorporated herein by reference in its entirety. In some embodiments, the linker is cleavable.

TABLE 2
Exemplary Glycoproteins.

| Uniprot | Protein Name |
|---|---|
| P01023 | Alpha-2-macroglobulin (Alpha-2-M) (C3 and PZP-like alpha-2-macroglobulin |
| | domain-containing protein 5) |
| A8K2U0 | Alpha-2-macroglobulin-like protein 1 (C3 and PZP-like alpha-2-macroglobulin |
| | domain-containing protein 9) |
| Q9BZC7 | ATP-binding cassette sub-family A member 2 (ATP-binding cassette transporter |
| | 2) (ATP-binding cassette 2) |
| Q99758 | ATP-binding cassette sub-family A member 3 (ABC-C transporter) (ATP- |
| | binding cassette transporter 3) (ATP-binding cassette 3) |
| Q8WWZ7 | ATP-binding cassette sub-family A member 5 |
| Q8IZY2 | ATP-binding cassette sub-family A member 7 (ABCA-SSN) (Autoantige?S-N) |
| | (Macrophage ABC transporter) |
| O94911 | ATP-binding cassette sub-family A member 8 |
| P08183 | Multidrug resistance protein 1 (EC 3.6.3.44) (ATP-binding cassette sub-family |
| | B member 1) (P-glycoprotein 1) (CD antigen CD243) |
| Q9UNQ0 | ATP-binding cassette sub-family G member 2 (Breast cancer resistance protein) |
| | (CDw338) (Mitoxantrone resistance-associated protein) (Placenta-specific ATP- |
| | binding cassette transporter) (Urate exporter) (CD antigen CD338) |
| Q7Z7G0 | Target of Nesh-SH3 (Tarsh) (ABI gene family member 3-binding protein) |
| | (Nesh-binding protein) (NeshBP) |
| P42765 | 3-ketoacyl-CoA thiolase, mitochondrial (EC 2.3.1.16) (Acetyl-CoA |
| | acyltransferase) (Beta-ketothiolase) (Mitochondrial 3-oxoacyl-CoA thiolase) |
| | (T1) |
| P16112 | Aggrecan core protein (Cartilage-specific proteoglycan core protein) (CSPCP) |
| | (Chondroitin sulfate proteoglycan core protein 1) (Chondroitin sulfate |
| | proteoglycan 1) [Cleaved into: Aggrecan core protein 2] |
| P24752 | Acetyl-CoA acetyltransferase, mitochondrial (EC 2.3.1.9) (Acetoacetyl-CoA |
| | thiolase) (T2) |
| Q5T8D3 | Acyl-CoA-binding domain-containing protein 5 |
| P12821 | Angiotensin-converting enzyme (ACE) (EC 3.2.1.—) (EC 3.4.15.1) (Dipeptidyl |
| | carboxypeptidase I) (Kininase II) (CD antigen CD143) [Cleaved into: |
| | Angiotensin-converting enzyme, soluble form] |
| Q99798 | Aconitate hydratase, mitochondrial (Aconitase) (EC 4.2.1.3) (Citrate |
| | hydrolyase) |
| P11117 | Lysosomal acid phosphatase (LAP) (EC 3.1.3.2) |
| O14672 | Disintegrin and metalloproteinase domain-containing protein 10 (ADAM 10) |
| | (EC 3.4.24.81) (CDw156) (Kuzbanian protein homolog) (Mammalian |
| | disintegrin-metalloprotease) (CD antigen CD156c) |
| O75078 | Disintegrin and metalloproteinase domain-containing protein 11 (ADAM 11) |
| | (Metalloproteinase-like, disintegrin-like, and cysteine-rich protein) (MDC) |
| P78536 | Disintegrin and metalloproteinase domain-containing protein 17 (ADAM 17) |
| | (EC 3.4.24.86) (Snake venom-like protease) (TNF-alpha convertase) (TNF- |
| | alpha-converting enzyme) (CD antigen CD156b) |
| Q9P0K1 | Disintegrin and metalloproteinase domain-containing protein 22 (ADAM 22) |
| | (Metalloproteinase-disintegrin ADAM22-3) (Metalloproteinase-like, disintegrin- |
| | like, and cysteine-rich protein 2) |
| O75077 | Disintegrin and metalloproteinase domain-containing protein 23 (ADAM 23) |
| | (Metalloproteinase-like, disintegrin-like, and cysteine-rich protein 3) (MDC-3) |
| Q6NVV9 | Putative disintegrin and metalloproteinase domain-containing protein 5 (Putative |
| | transmembrane metalloproteinase-like, disintegrin-like, and cysteine-rich protein |
| | II) (tMDC II) |
| Q13443 | Disintegrin and metalloproteinase domain-containing protein 9 (ADAM 9) (EC |
| | 3.4.24.—) (Cellular disintegrin-related protein) (Meltrin-gamma) |
| | (Metalloprotease/disintegrin/cysteine-rich protein 9) (Myeloma cell |
| | metalloproteinase) |
| O14514 | Adhesion G protein-coupled receptor B1 (Brain-specific angiogenesis inhibitor |
| | 1) [Cleaved into: Vasculostatin-40 (Vstat40); Vasculostatin-120 (Vstat120)] |
| O60242 | Adhesion G protein-coupled receptor B3 (Brain-specific angiogenesis inhibitor |
| | 3) |
| Q8IZF2 | Adhesion G protein-coupled receptor F5 (G-protein coupled receptor 116) |
| Q9Y653 | Adhesion G-protein coupled receptor G1 (G-protein coupled receptor 56) |
| | (Protein TM7XN1) [Cleaved into: ADGRG1 N-terminal fragment (ADGRG1 |
| | NT) (GPR56 N-terminal fragment) (GPR56 NT) (GPR56(N)) (GPR56 |
| | extracellular subunit) (GPR56 subunit alpha); ADGRG1 C-terminal fragment |
| | (ADGRG1 CT) (GPR56 C-terminal fragment) (GPR56 CT) (GPR56(C)) |
| | (GPR56 seven-transmembrane subunit) (GPR56 7TM) (GPR56 subunit beta)] |
| O94910 | Adhesion G protein-coupled receptor L1 (Calcium-independent alpha-latrotoxin |
| | receptor 1) (CIRL-1) (Latrophilin-1) (Lectomedin-2) |
| O95490 | Adhesion G protein-coupled receptor L2 (Calcium-independent alpha-latrotoxin |
| | receptor 2) (CIRL-2) (Latrophilin homolog 1) (Latrophilin-2) (Lectomedin-1) |
| Q9HAR2 | Adhesion G protein-coupled receptor L3 (Calcium-independent alpha-latrotoxin |
| | receptor 3) (CIRL-3) (Latrophilin-3) (Lectomedin-3) |
| Q9HBW9 | Adhesion G protein-coupled receptor L4 (EGF, latrophilin and seven |
| | transmembrane domain-containing protein 1) (EGF-TM7-latrophilin-related |
| | protein) (ETL protein) |
| P30542 | Adenosine receptor A1 |
| Q8IUX7 | Adipocyte enhancer-binding protein 1 (AE-binding protein 1) (Aortic |
| | carboxypeptidase-like protein) |
| Q6ZNB7 | Alkylglycerol monooxygenase (EC 1.14.16.5) (Transmembrane protein 195) |
| O00468 | Agrin [Cleaved into: Agrin N-terminal 110 kDa subunit; Agrin C-terminal 110 |
| | kDa subunit; Agrin C-terminal 90 kDa fragment (C90); Agrin C-terminal 22 |
| | kDa fragment (C22)] |
| O43865 | S-adenosylhomocysteine hydrolase-like protein 1 (DC-expressed AHCY-like |
| | molecule) (IP(3)Rs binding protein released with IP(3)) (IRBIT) (Putative |
| | adenosylhomocysteinase 2) (S-adenosyl-L-homocysteine hydrolase 2) |
| | (AdoHcyase 2) |
| P02765 | Alpha-2-HS-glycoprotein (Alpha-2-Z-globulin) (Ba-alpha-2-glycoprotein) |
| | (Fetuin-A) [Cleaved into: Alpha-2-HS-glycoprotein chain A; Alpha-2-HS- |
| | glycoprotein chain B] |
| P12763 | Alpha-2-HS-glycoprotein (Asialofetuin) (Fetuin-A) |
| Q13023 | A-kinase anchor protein 6 (AKAP-6) (A-kinase anchor protein 100 kDa) (AKAP |
| | 100) (Protein kinase A-anchoring protein 6) (PRKA6) (mAKAP) |
| Q13740 | CD166 antigen (Activated leukocyte cell adhesion molecule) (CD antigen |
| | CD166) |
| P05091 | Aldehyde dehydrogenase, mitochondrial (EC 1.2.1.3) (ALDH class 2) (ALDH- |
| | E2) (ALDHI) |
| P30038 | Delta-1-pyrroline-5-carboxylate dehydrogenase, mitochondrial (P5C |
| | dehydrogenase) (EC 1.2.1.88) (Aldehyde dehydrogenase family 4 member A1) |
| | (L-glutamate gamma-semialdehyde dehydrogenase) |
| P51649 | Succinate-semialdehyde dehydrogenase, mitochondrial (EC 1.2.1.24) (Aldehyde |
| | dehydrogenase family 5 member A1) (NAD(+)-dependent succinic |
| | semialdehyde dehydrogenase) |
| Q02252 | Methylmalonate-semialdehyde dehydrogenase [acylating], mitochondrial |
| | (MMSDH) (Malonate-semialdehyde dehydrogenase [acylating]) (EC 1.2.1.18) |
| | (EC 1.2.1.27) (Aldehyde dehydrogenase family 6 member A1) |
| P49189 | 4-trimethylaminobutyraldehyde dehydrogenase (TMABADH) (EC 1.2.1.47) |
| | (Aldehyde dehydrogenase E3 isozyme) (Aldehyde dehydrogenase family 9 |
| | member A1) (EC 1.2.1.3) (Gamma-aminobutyraldehyde dehydrogenase) (EC |
| | 1.2.1.19) (R-aminobutyraldehyde dehydrogenase) [Cleaved into: 4- |
| | trimethylaminobutyraldehyde dehydrogenase, N-terminally processed] |
| Q9UM73 | ALK tyrosine kinase receptor (EC 2.7.10.1) (Anaplastic lymphoma kinase) (CD |
| | antigen CD246) |
| P05186 | Alkaline phosphatase, tissue-nonspecific isozyme (AP-TNAP) (TNSALP) (EC |
| | 3.1.3.1) (Alkaline phosphatase liver/bone/kidney isozyme) |
| Q86WK6 | Amphoterin-induced protein 1 (AMIGO-1) (Alivin-2) |
| Q9BYT9 | Anoctamin-3 (Transmembrane protein 16C) |
| Q4KMQ2 | Anoctamin-6 (Small-conductance calcium-activated nonselective cation |
| | channel) (SCAN channel) (Transmembrane protein 16F) |
| P15144 | Aminopeptidase N (AP-N) (hAPN) (EC 3.4.11.2) (Alanyl aminopeptidase) |
| | (Aminopeptidase M) (AP-M) (Microsomal aminopeptidase) (Myeloid plasma |
| | membrane glycoprotein CD13) (gp150) (CD antigen CD13) |
| Q16853 | Membrane primary amine oxidase (EC 1.4.3.21) (Copper amine oxidase) |
| | (HPAO) (Semicarbazide-sensitive amine oxidase) (SSAO) (Vascular adhesion |
| | protein 1) (VAP-1) |
| P63010 | AP-2 complex subunit beta (AP105B) (Adaptor protein complex AP-2 subunit |
| | beta) (Adaptor-related protein complex 2 subunit beta) (Beta-2-adaptin) (Beta- |
| | adaptin) (Clathrin assembly protein complex 2 beta large chain) (Plasma |
| | membrane adaptor HA2/AP2 adaptin beta subunit) |
| P25054 | Adenomatous polyposis coli protein (Protein APC) (Deleted in polyposis 2.5) |
| Q9UBZ4 | DNA-(apurinic or apyrimidinic site) lyase 2 (EC 3.1.—.—) (EC 4.2.99.18) (AP |
| | endonuclease XTH2) (APEX nuclease 2) (APEX nuclease-like 2) (Apurinic- |
| | apyrimidinic endonuclease 2) (AP endonuclease 2) |
| P51693 | Amyloid-like protein 1 (APLP) (APLP-1) [Cleaved into: C30] |
| Q06481 | Amyloid-like protein 2 (APLP-2) (APPH) (Amyloid protein homolog) (CDEI |
| | box-binding protein) (CDEBP) |
| Q9HDC9 | Adipocyte plasma membrane-associated protein (Protein BSCv) |
| P04114 | Apolipoprotein B-100 (Apo B-100) [Cleaved into: Apolipoprotein B-48 (Apo B- |
| | 48)] |
| P05090 | Apolipoprotein D (Apo-D) (ApoD) |
| P02749 | Beta-2-glycoprotein 1 (APC inhibitor) (Activated protein C-binding protein) |
| | (Anticardiolipin cofactor) (Apolipoprotein H) (Apo-H) (Beta-2-glycoprotein I) |
| | (B2GPI) (Beta(2)GPI) |
| O95445 | Apolipoprotein M (Apo-M) (ApoM) (Protein G3a) |
| P05067 | Amyloid-beta A4 protein (ABPP) (APPI) (APP) (Alzheimer disease amyloid |
| | protein) (Amyloid precursor protein) (Amyloid-beta precursor protein) (Cerebral |
| | vascular amyloid peptide) (CVAP) (PreA4) (Protease nexin-II) (PN-II) [Cleaved |
| | into: N-APP; Soluble APP-alpha (S-APP-alpha); Soluble APP-beta (S-APP- |
| | beta); C99 (Beta-secretase C-terminal fragment) (Beta-CTF); Amyloid-beta |
| | protein 42 (Abeta42) (Beta-APP42); Amyloid-beta protein 40 (Abeta40) (Beta- |
| | APP40); C83 (Alpha-secretase C-terminal fragment) (Alpha-CTF); P3(42); |
| | P3(40); C80; Gamma-secretase C-terminal fragment 59 (Amyloid intracellular |
| | domain 59) (AICD-59) (AID(59)) (Gamma-CTF(59)); Gamma-secretase C- |
| | terminal fragment 57 (Amyloid intracellular domain 57) (AICD-57) (AID(57)) |
| | (Gamma-CTF(57)); Gamma-secretase C-terminal fragment 50 (Amyloid |
| | intracellular domain 50) (AICD-50) (AID(50)) (Gamma-CTF(50)); C31] |
| P29972 | Aquaporin-1 (AQP-1) (Aquaporin-CHIP) (Urine water channel) (Water channel |
| | protein for red blood cells and kidney proximal tubule) |
| P55087 | Aquaporin-4 (AQP-4) (Mercurial-insensitive water channel) (MIWC) (WCH4) |
| P59998 | Actin-related protein 2/3 complex subunit 4 (Arp2/3 complex 20 kDa subunit) |
| | (p20-ARC) |
| P15848 | Arylsulfatase B (ASB) (EC 3.1.6.12) (N-acetylgalactosamine-4-sulfatase) (G4S) |
| P51689 | Arylsulfatase D (ASD) (EC 3.1.6.—) |
| P54793 | Arylsulfatase F (ASF) (EC 3.1.6.—) |
| Q96EG1 | Arylsulfatase G (ASG) (EC 3.1.6.—) |
| Q13510 | Acid ceramidase (AC) (ACDase) (Acid CDase) (EC 3.5.1.23) (Acylsphingosine |
| | deacylase) (N-acylsphingosine amidohydrolase) (Putative 32 kDa heart protein) |
| | (PHP32) [Cleaved into: Acid ceramidase subunit alpha; Acid ceramidase subunit |
| | beta] |
| P78348 | Acid-sensing ion channel 1 (ASIC1) (Amiloride-sensitive cation channel 2, |
| | neuronal) (Brain sodium channel 2) (BNaC2) |
| Q96FT7 | Acid-sensing ion channel 4 (ASIC4) (Amiloride-sensitive cation channel 4) |
| | (Amiloride-sensitive cation channel 4, pituitary) |
| Q12797 | Aspartyl/asparaginyl beta-hydroxylase (EC 1.14.11.16) (Aspartate beta- |
| | hydroxylase) (ASP beta-hydroxylase) (Peptide-aspartate beta-dioxygenase) |
| Q9BXN1 | Asporin (Periodontal ligament-associated protein 1) (PLAP-1) |
| O14525 | Astrotactin-1 |
| O75129 | Astrotactin-2 |
| Q8NBU5 | ATPase family AAA domain-containing protein 1 (EC 3.6.1.3) (Thorase) |
| P05023 | Sodium/potassium-transporting ATPase subunit alpha-1 (Na(+)/K(+) ATPase |
| | alpha-1 subunit) (EC 3.6.3.9) (Sodium pump subunit alpha-1) |
| P50993 | Sodium/potassium-transporting ATPase subunit alpha-2 (Na(+)/K(+) ATPase |
| | alpha-2 subunit) (EC 3.6.3.9) (Sodium pump subunit alpha-2) |
| P13637 | Sodium/potassium-transporting ATPase subunit alpha-3 (Na(+)/K(+) ATPase |
| | alpha-3 subunit) (EC 3.6.3.9) (Na(+)/K(+) ATPase alpha(III) subunit) (Sodium |
| | pump subunit alpha-3) |
| P05026 | Sodium/potassium-transporting ATPase subunit beta-1 (Sodium/potassium- |
| | dependent ATPase subunit beta-1) |
| P14415 | Sodium/potassium-transporting ATPase subunit beta-2 (Adhesion molecule in |
| | glia) (AMOG) (Sodium/potassium-dependent ATPase subunit beta-2) |
| P54709 | Sodium/potassium-transporting ATPase subunit beta-3 (Sodium/potassium- |
| | dependent ATPase subunit beta-3) (ATPB-3) (CD antigen CD298) |
| P16615 | Sarcoplasmic/endoplasmic reticulum calcium ATPase 2 (SERCA2) (SR Ca(2+)- |
| | ATPase 2) (EC 3.6.3.8) (Calcium pump 2) (Calcium-transporting ATPase |
| | sarcoplasmic reticulum type, slow twitch skeletal muscle isoform) (Endoplasmic |
| | reticulum class 1/2 Ca(2+) ATPase) |
| P20020 | Plasma membrane calcium-transporting ATPase 1 (PMCA1) (EC 3.6.3.8) |
| | (Plasma membrane calcium ATPase isoform 1) (Plasma membrane calcium |
| | pump isoform 1) |
| P23634 | Plasma membrane calcium-transporting ATPase 4 (PMCA4) (EC 3.6.3.8) |
| | (Matrix-remodeling-associated protein 1) (Plasma membrane calcium ATPase |
| | isoform 4) (Plasma membrane calcium pump isoform 4) |
| Q15904 | V-type proton ATPase subunit S1 (V-ATPase subunit S1) (Protein XAP-3) (V- |
| | ATPase Ac45 subunit) (V-ATPase S1 accessory protein) (Vacuolar proton pump |
| | subunit S1) |
| O15342 | V-type proton ATPase subunit e 1 (V-ATPase subunit e 1) (V-ATPase 9.2 kDa |
| | membrane accessory protein) (V-ATPase M9.2 subunit) (Vacuolar proton pump |
| | subunit e 1) |
| Q8NHE4 | V-type proton ATPase subunit e 2 (V-ATPase subunit e 2) (Lysosomal 9 kDa |
| | H(+)-transporting ATPase V0 subunit e2) (Vacuolar proton pump subunit e 2) |
| P21281 | V-type proton ATPase subunit B, brain isoform (V-ATPase subunit B 2) |
| | (Endomembrane proton pump 58 kDa subunit) (HO57) (Vacuolar proton pump |
| | subunit B 2) |
| P21283 | V-type proton ATPase subunit C 1 (V-ATPase subunit C 1) (Vacuolar proton |
| | pump subunit C 1) |
| O95670 | V-type proton ATPase subunit G 2 (V-ATPase subunit G 2) (V-ATPase 13 kDa |
| | subunit 2) (Vacuolar proton pump subunit G 2) |
| O75882 | Attractin (DPPT-L) (Mahogany homolog) |
| Q5VV63 | Attractin-like protein 1 |
| P46100 | Transcriptional regulator ATRX (EC 3.6.4.12) (ATP-dependent helicase ATRX) |
| | (X-linked helicase II) (X-linked nuclear protein) (XNP) (Znf-HX) |
| P30530 | Tyrosine-protein kinase receptor UFO (EC 2.7.10.1) (AXL oncogene) |
| P25311 | Zinc-alpha-2-glycoprotein (Zn-alpha-2-GP) (Zn-alpha-2-glycoprotein) |
| P20160 | Azurocidin (Cationic antimicrobial protein CAP37) (Heparin-binding protein) |
| | (HBP) (hHBP) |
| O75752 | UDP-GalNAc: beta-1,3-N-acetylgalactosaminyltransferase 1 (Beta-1,3-GalNAc- |
| | T1) (EC 2.4.1.79) (Beta-1,3-galactosyltransferase 3) (Beta-1,3-GalTase 3) |
| | (Beta3Gal-T3) (Beta3GalT3) (b3Gal-T3) (Beta-3-Gx-T3) |
| | (Galactosylgalactosylglucosylceramide beta-D-acetyl- |
| | galactosaminyltransferase) (Globoside synthase) (UDP-N- |
| | acetylgalactosamine: globotriaosylceramidebeta-1,3-N- |
| | acetylgalactosaminyltransferase) |
| Q6Y288 | Beta-1,3-glucosyltransferase (Beta3Glc-T) (EC 2.4.1.—) (Beta 3- |
| | glucosyltransferase) (Beta-3-glycosyltransferase-like) |
| Q9NY97 | N-acetyllactosaminide beta-1,3-N-acetylglucosaminyltransferase 2 (EC |
| | 2.4.1.149) (Beta-1,3-N-acetylglucosaminyltransferase 1) (BGnT-1) (Beta-1,3- |
| | Gn-T1) (Beta3Gn-T1) (Beta-1,3-galactosyltransferase 7) (Beta-1,3-GalTase 7) |
| | (Beta3Gal-T7) (Beta3GalT7) (b3Gal-T7) (Beta-3-Gx-T7) (UDP-Gal: beta- |
| | GlcNAc beta-1,3-galactosyltransferase 7) (UDP-GlcNAc: betaGal beta-1,3-N- |
| | acetylglucosaminyltransferase 2) (BGnT-2) (Beta-1,3-Gn-T2) (Beta-1,3-N- |
| | acetylglucosaminyltransferase 2) (Beta3Gn-T2) (UDP-galactose: beta-N- |
| | acetylglucosamine beta-1,3-galactosyltransferase 7) |
| O43505 | Beta-1,4-glucuronyltransferase 1 (EC 2.4.1.—) (I-beta-1,3-N- |
| | acetylglucosaminyltransferase) (iGnT) (N-acetyllactosaminide beta-1,3-N- |
| | acetylglucosaminyltransferase) (Poly-N-acetyllactosamine extension enzyme) |
| | (UDP-GlcNAc: betaGal beta-1,3-N-acetylglucosaminyltransferase 1) |
| P56817 | Beta-secretase 1 (EC 3.4.23.46) (Aspartyl protease 2) (ASP2) (Asp 2) (Beta-site |
| | amyloid precursor protein cleaving enzyme 1) (Beta-site APP cleaving enzyme |
| | 1) (Memapsin-2) (Membrane-associated aspartic protease 2) |
| P50895 | Basal cell adhesion molecule (Auberger B antigen) (B-CAM cell surface |
| | glycoprotein) (F8/G253 antigen) (Lutheran antigen) (Lutheran blood group |
| | glycoprotein) (CD antigen CD239) |
| Q96GW7 | Brevican core protein (Brain-enriched hyaluronan-binding protein) (BEHAB) |
| | (Chondroitin sulfate proteoglycan 7) |
| P06276 | Cholinesterase (EC 3.1.1.8) (Acylcholine acylhydrolase) (Butyrylcholine |
| | esterase) (Choline esterase II) (Pseudocholinesterase) |
| Q9BXK5 | Bcl-2-like protein 13 (Bcl2-L-13) (Bcl-rambo) (Protein Mil1) |
| O00512 | B-cell CLL/lymphoma 9 protein (B-cell lymphoma 9 protein) (Bcl-9) (Protein |
| | legless homolog) |
| Q8N7W2 | BEN domain-containing protein 7 |
| P21810 | Biglycan (Bone/cartilage proteoglycan I) (PG-S1) |
| Q8N8U9 | BMP-binding endothelial regulator protein (Bone morphogenetic protein- |
| | binding endothelial cell precursor-derived regulator) (Protein crossveinless-2) |
| | (hCV2) |
| P51813 | Cytoplasmic tyrosine-protein kinase BMX (EC 2.7.10.2) (Bone marrow tyrosine |
| | kinase gene in chromosome X protein) (Epithelial and endothelial tyrosine |
| | kinase) (ETK) (NTK38) |
| Q9BWV1 | Brother of CDO (Protein BOC) |
| O60477 | BMP/retinoic acid-inducible neural-specific protein 1 (Deleted in bladder cancer |
| | protein 1) |
| Q9C0B6 | BMP/retinoic acid-inducible neural-specific protein 2 (DBCCR1-like protein 2) |
| Q76B58 | BMP/retinoic acid-inducible neural-specific protein 3 (DBCCR1-like protein 1) |
| Q96G97 | Seipin (Bernardinelli-Seip congenital lipodystrophy type 2 protein) |
| P35613 | Basigin (5F7) (Collagenase stimulatory factor) (Extracellular matrix |
| | metalloproteinase inducer) (EMMPRIN) (Leukocyte activation antigen M6) |
| | (OK blood group antigen) (Tumor cell-derived collagenase stimulatory factor) |
| | (TCSF) (CD antigen CD147) |
| Q10588 | ADP-ribosyl cyclase/cyclic ADP-ribose hydrolase 2 (EC 3.2.2.6) (ADP-ribosyl |
| | cyclase 2) (Bone marrow stromal antigen 1) (BST-1) (Cyclic ADP-ribose |
| | hydrolase 2) (cADPr hydrolase 2) (CD antigen CD157) |
| Q10589 | Bone marrow stromal antigen 2 (BST-2) (HM1.24 antigen) (Tetherin) (CD |
| | antigen CD317) |
| A6NE02 | BTB/POZ domain-containing protein 17 (Galectin-3-binding protein-like) |
| P43251 | Biotinidase (Biotinase) (EC 3.5.1.12) |
| P20290 | Transcription factor BTF3 (Nascent polypeptide-associated complex subunit |
| | beta) (NAC-beta) (RNA polymerase B transcription factor 3) |
| Q7KYR7 | Butyrophilin subfamily 2 member A1 |
| Q96F05 | Uncharacterized protein C11orf24 (Protein DM4E3) |
| P02745 | Complement C1q subcomponent subunit A |
| P00736 | Complement C1r subcomponent (EC 3.4.21.41) (Complement component 1 |
| | subcomponent r) [Cleaved into: Complement C1r subcomponent heavy chain; |
| | Complement C1r subcomponent light chain] |
| P09871 | Complement C1s subcomponent (EC 3.4.21.42) (C1 esterase) (Complement |
| | component 1 subcomponent s) [Cleaved into: Complement C1s subcomponent |
| | heavy chain; Complement C1s subcomponent light chain] |
| P06681 | Complement C2 (EC 3.4.21.43) (C3/C5 convertase) [Cleaved into: Complement |
| | C2b fragment; Complement C2a fragment] |
| P01024 | Complement C3 (C3 and PZP-like alpha-2-macroglobulin domain-containing |
| | protein 1) [Cleaved into: Complement C3 beta chain; C3-beta-c (C3bc); |
| | Complement C3 alpha chain; C3a anaphylatoxin; Acylation stimulating protein |
| | (ASP) (C3adesArg); Complement C3b alpha′ chain; Complement C3c alpha′ |
| | chain fragment 1; Complement C3dg fragment; Complement C3g fragment; |
| | Complement C3d fragment; Complement C3f fragment; Complement C3c alpha′ |
| | chain fragment 2] |
| P0C0L4 | Complement C4-A (Acidic complement C4) (C3 and PZP-like alpha-2- |
| | macroglobulin domain-containing protein 2) [Cleaved into: Complement C4 |
| | beta chain; Complement C4-A alpha chain; C4a anaphylatoxin; C4b-A; C4d-A; |
| | Complement C4 gamma chain] |
| P04003 | C4b-binding protein alpha chain (C4bp) (Proline-rich protein) (PRP) |
| P20851 | C4b-binding protein beta chain |
| Q9NS85 | Carbonic anhydrase-related protein 10 (Carbonic anhydrase-related protein X) |
| | (CA-RP X) (CARP X) (Cerebral protein 15) |
| Q5VU97 | VWFA and cache domain-containing protein 1 (Cache domain-containing |
| | protein 1) |
| Q00975 | Voltage-dependent N-type calcium channel subunit alpha-1B (Brain calcium |
| | channel III) (BIII) (Calcium channel, L type, alpha-1 polypeptide isoform 5) |
| | (Voltage-gated calcium channel subunit alpha Cav2.2) |
| Q13936 | Voltage-dependent L-type calcium channel subunit alpha-1C (Calcium channel, |
| | L type, alpha-1 polypeptide, isoform 1, cardiac muscle) (Voltage-gated calcium |
| | channel subunit alpha Cav1.2) |
| P54289 | Voltage-dependent calcium channel subunit alpha-2/delta-1 (Voltage-gated |
| | calcium channel subunit alpha-2/delta-1) [Cleaved into: Voltage-dependent |
| | calcium channel subunit alpha-2-1; Voltage-dependent calcium channel subunit |
| | delta-1] |
| Q9NY47 | Voltage-dependent calcium channel subunit alpha-2/delta-2 (Voltage-gated |
| | calcium channel subunit alpha-2/delta-2) [Cleaved into: Voltage-dependent |
| | calcium channel subunit alpha-2-2; Voltage-dependent calcium channel subunit |
| | delta-2] |
| Q8IZS8 | Voltage-dependent calcium channel subunit alpha-2/delta-3 (Voltage-gated |
| | calcium channel subunit alpha-2/delta-3) [Cleaved into: Voltage-dependent |
| | calcium channel subunit alpha-2-3; Voltage-dependent calcium channel subunit |
| | delta-3] |
| Q8WXS5 | Voltage-dependent calcium channel gamma-8 subunit (Neuronal voltage-gated |
| | calcium channel gamma-8 subunit) (Transmembrane AMPAR regulatory protein |
| | gamma-8) (TARP gamma-8) |
| Q9BY67 | Cell adhesion molecule 1 (Immunoglobulin superfamily member 4) (IgSF4) |
| | (Nectin-like protein 2) (NECL-2) (Spermatogenic immunoglobulin superfamily) |
| | (SgIgSF) (Synaptic cell adhesion molecule) (SynCAM) (Tumor suppressor in |
| | lung cancer 1) (TSLC-1) |
| Q8N3J6 | Cell adhesion molecule 2 (Immunoglobulin superfamily member 4D) (IgSF4D) |
| | (Nectin-like protein 3) (NECL-3) (Synaptic cell adhesion molecule 2) (SynCAM |
| | 2) |
| Q8NFZ8 | Cell adhesion molecule 4 (Immunoglobulin superfamily member 4C) (IgSF4C) |
| | (Nectin-like protein 4) (NECL-4) (TSLC1-like protein 2) |
| Q16602 | Calcitonin gene-related peptide type 1 receptor (CGRP type 1 receptor) |
| | (Calcitonin receptor-like receptor) |
| Q9NZT1 | Calmodulin-like protein 5 (Calmodulin-like skin protein) |
| P27797 | Calreticulin (CRP55) (Calregulin) (Endoplasmic reticulum resident protein 60) |
| | (ERp60) (HACBP) (grp60) |
| O43852 | Calumenin (Crocalbin) (IEF SSP 9302) |
| Q13557 | Calcium/calmodulin-dependent protein kinase type II subunit delta (CaM kinase |
| | II subunit delta) (CaMK-II subunit delta) (EC 2.7.11.17) |
| Q6P4E1 | Protein CASC4 (Cancer susceptibility candidate gene 4 protein) |
| Q96PB1 | N-acetylneuraminate 9-O-acetyltransferase (EC 2.3.1.45) (CAS1 domain- |
| | containing protein 1) (Sialate O-acetyltransferase) (SOAT) |
| Q9UKL3 | CASP8-associated protein 2 (FLICE-associated huge protein) |
| Q8IUK8 | Cerebellin-2 |
| Q8TBZ0 | Coiled-coil domain-containing protein 110 (Cancer/testis antigen 52) (CT52) |
| | (Cancer/testis antigen KM-HN-1) |
| Q7Z6B0 | Coiled-coil domain-containing protein 91 (GGA-binding partner) (p56 accessory |
| | protein) |
| O14618 | Copper chaperone for superoxide dismutase (Superoxide dismutase copper |
| | chaperone) |
| Q6YHK3 | CD109 antigen (150 kDa TGF-beta-1-binding protein) (C3 and PZP-like alpha- |
| | 2-macroglobulin domain-containing protein 7) (Platelet-specific Gov antigen) |
| | (p180) (r150) (CD antigen CD109) |
| P08571 | Monocyte differentiation antigen CD14 (Myeloid cell-specific leucine-rich |
| | glycoprotein) (CD antigen CD14) [Cleaved into: Monocyte differentiation |
| | antigen CD14, urinary form; Monocyte differentiation antigen CD14, |
| | membrane-bound form] |
| P48509 | CD151 antigen (GP27) (Membrane glycoprotei?FA-1) (Platelet-endothelial |
| | tetraspan antigen 3) (PETA-3) (Tetraspanin-24) (Tspan-24) (CD antigen CD151) |
| Q86VB7 | Scavenger receptor cysteine-rich type 1 protein M130 (Hemoglobin scavenger |
| | receptor) (CD antigen CD163) [Cleaved into: Soluble CD163 (sCD163)] |
| Q8N6Q3 | CD177 antigen (Human neutrophil alloantigen 2a) (HNA-2a) (NB1 |
| | glycoprotein) (NB1 GP) (Polycythemia rubra vera protein 1) (PRV-1) (CD |
| | antigen CD177) |
| Q99467 | CD180 antigen (Lymphocyte antigen 64) (Radioprotective 105 kDa protein) |
| | (CD antigen CD180) |
| P41217 | OX-2 membrane glycoprotein (CD antigen CD200) |
| Q9NZQ7 | Programmed cell death 1 ligand 1 (PD-L1) (PDCD1 ligand 1) (Programmed |
| | death ligand 1) (B7 homolog 1) (B7-H1) (CD antigen CD274) |
| Q5ZPR3 | CD276 antigen (4Ig-B7-H3) (B7 homolog 3) (B7-H3) (Costimulatory molecule) |
| | (CD antigen CD276) |
| P20138 | Myeloid cell surface antigen CD33 (Sialic acid-binding Ig-like lectin 3) (Siglec- |
| | 3) (gp67) (CD antigen CD33) |
| P16671 | Platelet glycoprotein 4 (Fatty acid translocase) (FAT) (Glycoprotein IIIb) |
| | (GPIIIB) (Leukocyte differentiation antigen CD36) (PAS IV) (PAS-4) (Platelet |
| | collagen receptor) (Platelet glycoprotein IV) (GPIV) (Thrombospondin receptor) |
| | (CD antigen CD36) |
| P28907 | ADP-ribosyl cyclase/cyclic ADP-ribose hydrolase 1 (EC 3.2.2.6) (2′-phospho- |
| | ADP-ribosyl cyclase) (2′-phospho-ADP-ribosyl cyclase/2′-phospho-cyclic-ADP- |
| | ribose transferase) (EC 2.4.99.20) (2′-phospho-cyclic-ADP-ribose transferase) |
| | (ADP-ribosyl cyclase 1) (ADPRC 1) (Cyclic ADP-ribose hydrolase 1) (cADPr |
| | hydrolase 1) (T10) (CD antigen CD38) |
| P16070 | CD44 antigen (CDw44) (Epican) (Extracellular matrix receptor III) (ECMR-III) |
| | (GP90 lymphocyte homing/adhesion receptor) (HUTCH-I) (Heparan sulfate |
| | proteoglycan) (Hermes antigen) (Hyaluronate receptor) (Phagocytic |
| | glycoprotein 1) (PGP-1) (Phagocytic glycoprotein I) (PGP-I) (CD antigen |
| | CD44) |
| P15529 | Membrane cofactor protein (TLX) (Trophoblast leukocyte common antigen) |
| | (CD antigen CD46) |
| Q08722 | Leukocyte surface antigen CD47 (Antigenic surface determinant protein OA3) |
| | (Integrin-associated protein) (IAP) (Protein MER6) (CD antigen CD47) |
| P19397 | Leukocyte surface antigen CD53 (Cell surface glycoprotein CD53) |
| | (Tetraspanin-25) (Tspan-25) (CD antigen CD53) |
| P19256 | Lymphocyte function-associated antigen 3 (Ag3) (Surface glycoprotein LFA-3) |
| | (CD antigen CD58) |
| P13987 | CD59 glycoprotein (1F5 antigen) (20 kDa homologous restriction factor) (HRF- |
| | 20) (HRF20) (MAC-inhibitory protein) (MAC-IP) (MEM43 antigen) |
| | (Membrane attack complex inhibition factor) (MACIF) (Membrane inhibitor of |
| | reactive lysis) (MIRL) (Protectin) (CD antigen CD59) |
| P08962 | CD63 antigen (Granulophysin) (Lysosomal-associated membrane protein 3) |
| | (LAMP-3) (Melanoma-associated antigen ME491) (OMA81H) (Ocular |
| | melanoma-associated antigen) (Tetraspanin-30) (Tspan-30) (CD antigen CD63) |
| P34810 | Macrosialin (Gp110) (CD antigen CD68) |
| P04233 | HLA class II histocompatibility antigen gamma chain (HLA-DR antigens- |
| | associated invariant chain) (Ia antigen-associated invariant chain) (Ii) (p33) (CD |
| | antigen CD74) |
| P60033 | CD81 antigen (26 kDa cell surface protein TAPA-1) (Target of the |
| | antiproliferative antibody 1) (Tetraspanin-28) (Tspan-28) (CD antigen CD81) |
| P27701 | CD82 antigen (C33 antigen) (IA4) (Inducible membrane protein R2) (Metastasis |
| | suppressor Kangai-1) (Suppressor of turnorigenicity 6 protein) (Tetraspanin-27) |
| | (Tspan-27) (CD antigen CD82) |
| P21926 | CD9 antigen (5H9 antigen) (Cell growth-inhibiting gene 2 protein) (Leukocyte |
| | antigen MIC3) (Motility-related protein) (MRP-1) (Tetraspanin-29) (Tspan-29) |
| | (p24) (CD antigen CD9) |
| P48960 | CD97 antigen (Leukocyte antigen CD97) (CD antigen CD97) [Cleaved into: |
| | CD97 antigen subunit alpha; CD97 antigen subunit beta] |
| Q9Y6N8 | Cadherin-10 (T2-cadherin) |
| P55287 | Cadherin-11 (OSF-4) (Osteoblast cadherin) (OB-cadherin) |
| P55290 | Cadherin-13 (Heart cadherin) (H-cadherin) (P105) (Truncated cadherin) (T-cad) |
| | (T-cadherin) |
| Q13634 | Cadherin-18 (Cadherin-14) |
| P19022 | Cadherin-2 (CDw325) (Neural cadherin) (N-cadherin) (CD antigen CD325) |
| Q9HBT6 | Cadherin-20 |
| P55283 | Cadherin-4 (Retinal cadherin) (R-CAD) (R-cadherin) |
| P55285 | Cadherin-6 (Kidney cadherin) (K-cadherin) |
| P55286 | Cadherin-8 |
| Q9ULB4 | Cadherin-9 |
| O95674 | Phosphatidate cytidylyltransferase 2 (EC 2.7.7.41) (CDP-DAG synthase 2) |
| | (CDP-DG synthase 2) (CDP-diacylglycerol synthase 2) (CDS 2) (CDP- |
| | diglyceride pyrophosphorylase 2) (CDP-diglyceride synthase 2) |
| | (CTP: phosphatidate cytidylyltransferase 2) |
| Q9HCU4 | Cadherin EGF LAG seven-pass G-type receptor 2 (Cadherin family member 10) |
| | (Epidermal growth factor-like protein 2) (EGF-like protein 2) (Flamingo |
| | homolog 3) (Multiple epidermal growth factor-like domains protein 3) (Multiple |
| | EGF-like domains protein 3) |
| Q9HC77 | Centromere protein J (CENP-J) (Centrosomal P4.1-associated protein) (LAG-3- |
| | associated protein) (LYST-interacting protein 1) |
| Q96G23 | Ceramide synthase 2 (CerS2) (LAG1 longevity assurance homolog 2) (SP260) |
| | (Tumor metastasis-suppressor gene 1 protein) |
| Q6ZMG9 | Ceramide synthase 6 (CerS6) (LAG1 longevity assurance homolog 6) |
| P23141 | Liver carboxylesterase 1 (Acyl-coenzyme A: cholesterol acyltransferase) |
| | (ACAT) (Brain carboxylesterase hBr1) (Carboxylesterase 1) (CE-1) (hCE-1) |
| | (EC 3.1.1.1) (Cocaine carboxylesterase) (Egasyn) (HMSE) (Methylumbelliferyl- |
| | acetate deacetylase 1) (EC 3.1.1.56) (Monocyte/macrophage serine esterase) |
| | (Retinyl ester hydrolase) (REH) (Serine esterase 1) (Triacylglycerol hydrolase) |
| | (TGH) |
| Q6NT32 | Carboxylesterase 5A (EC 3.1.1.1) (Carboxylesterase-like urinary excreted |
| | protein homolog) (Cauxin) |
| P08603 | Complement factor H (H factor 1) |
| P36980 | Complement factor H-related protein 2 (FHR-2) (DDESK59) (H factor-like 3) |
| | (H factor-like protein 2) |
| O00533 | Neural cell adhesion molecule L1-like protein (Close homolog of L1) [Cleaved |
| | into: Processed neural cell adhesion molecule L1-like protein] |
| Q9UDT6 | CAP-Gly domain-containing linker protein 2 (Cytoplasmic linker protein 115) |
| | (CLIP-115) (Cytoplasmic linker protein 2) (Williams-Beuren syndrome |
| | chromosomal region 3 protein) (Williams-Beuren syndrome chromosomal |
| | region 4 protein) |
| O75503 | Ceroid-lipofuscinosis neuronal protein 5 (Protein CLN5) [Cleaved into: Ceroid- |
| | lipofuscinosis neuronal protein 5, secreted form] |
| O96005 | Cleft lip and palate transmembrane protein 1 |
| Q00610 | Clathrin heavy chain 1 (Clathrin heavy chain on chromosome 17) (CLH-17) |
| P10909 | Clusterin (Aging-associated gene 4 protein) (Apolipoprotein J) (Apo-J) |
| | (Complement cytolysis inhibitor) (CLI) (Complement-associated protein SP- |
| | 40,40) (Ku70-binding protein 1) (NA1/NA2) (Testosterone-repressed prostate |
| | message 2) (TRPM-2) [Cleaved into: Clusterin beta chain (ApoJalpha) |
| | (Complement cytolysis inhibitor a chain); Clusterin alpha chain (ApoJbeta) |
| | (Complement cytolysis inhibitor b chain)] |
| P10909 | Clusterin (Aging-associated gene 4 protein) (Apolipoprotein J) (Apo-J) |
| | (Complement cytolysis inhibitor) (CLI) (Complement-associated protei?P- |
| | 40,40) (Ku70-binding protein 1) (NA1/NA2) (Testosterone-repressed prostate |
| | message 2) (TRPM-2) [Cleaved into: Clusterin beta chain (ApoJalpha) |
| | (Complement cytolysis inhibitor a chain); Clusterin alpha chain (ApoJbeta) |
| | (Complement cytolysis inhibitor b chain)] |
| Q96AJ1 | Clusterin-associated protein 1 (Qilin) |
| Q96KN2 | Beta-Ala-His dipeptidase (EC 3.4.13.20) (CNDP dipeptidase 1) (Carnosine |
| | dipeptidase 1) (Glutamate carboxypeptidase-like protein 2) (Serum carnosinase) |
| Q16280 | Cyclic nucleotide-gated olfactory channel (Cyclic nucleotide-gated cation |
| | channel 2) (Cyclic nucleotide-gated channel alpha-2) (CNG channel alpha-2) |
| | (CNG-2) (CNG2) |
| Q9H8M5 | Metal transporter CNNM2 (Ancient conserved domain-containing protein 2) |
| | (Cyclin-M2) |
| Q8NE01 | Metal transporter CNNM3 (Ancient conserved domain-containing protein 3) |
| | (Cyclin-M3) |
| Q6P4Q7 | Metal transporter CNNM4 (Ancient conserved domain-containing protein 4) |
| | (Cyclin-M4) |
| Q9UIV1 | CCR4-NOT transcription complex subunit 7 (EC 3.1.13.4) (BTG1-binding |
| | factor 1) (CCR4-associated factor 1) (CAF-1) (Caf1a) |
| Q9BT09 | Protein canopy homolog 3 (CTG repeat protein 4a) (Expanded repeat-domain |
| | protein CAG/CTG 5) (Protein associated with TLR4) (Trinucleotide repeat- |
| | containing gene 5 protein) |
| P21554 | Cannabinoid receptor 1 (CB-R) (CB1) (CANN6) |
| Q96F85 | CB1 cannabinoid receptor-interacting protein 1 (CRIP-1) |
| Q12860 | Contactin-1 (Glycoprotein gp135) (Neural cell surface protein F3) |
| Q02246 | Contactin-2 (Axonal glycoprotein TAG-1) (Axonin-1) (Transient axonal |
| | glycoprotein 1) (TAX-1) |
| Q9P232 | Contactin-3 (Brain-derived immunoglobulin superfamily protein 1) (BIG-1) |
| | (Plasmacytoma-associated neuronal glycoprotein) |
| Q8IWV2 | Contactin-4 (Brain-derived immunoglobulin superfamily protein 2) (BIG-2) |
| O94779 | Contactin-5 (Neural recognition molecule NB-2) (hNB-2) |
| Q9UQ52 | Contactin-6 (Neural recognition molecule NB-3) (hNB-3) |
| P78357 | Contactin-associated protein 1 (Caspr) (Caspr1) (Neurexin IV) (Neurexin-4) |
| | (p190) |
| Q9UHC6 | Contactin-associated protein-like 2 (Cell recognition molecule Caspr2) |
| Q9C0A0 | Contactin-associated protein-like 4 (Cell recognition molecule Caspr4) |
| Q8WYK1 | Contactin-associated protein-like 5 (Cell recognition molecule Caspr5) |
| Q99715 | Collagen alpha-1(XII) chain |
| Q05707 | Collagen alpha-1(XIV) chain (Undulin) |
| Q96P44 | Collagen alpha-1(XXI) chain |
| P12109 | Collagen alpha-1(VI) chain |
| P12110 | Collagen alpha-2(VI) chain |
| P12111 | Collagen alpha-3(VI) chain |
| Q5KU26 | Collectin-12 (Collectin placenta protein 1) (CL-P1) (hCL-P1) (Nurse cell |
| | scavenger receptor 2) (Scavenger receptor class A member 4) (Scavenger |
| | receptor with C-type lectin) |
| Q8NBJ5 | Procollagen galactosyltransferase 1 (EC 2.4.1.50) (Collagen beta(1- |
| | O)galactosyltransferase 1) (ColGalT 1) (Glycosyltransferase 25 family member |
| | 1) (Hydroxylysine galactosyltransferase 1) |
| P00450 | Ceruloplasmin (EC 1.16.3.1) (Ferroxidase) |
| O75976 | Carboxypeptidase D (EC 3.4.17.22) (Metallocarboxypeptidase D) (gp180) |
| P16870 | Carboxypeptidase E (CPE) (EC 3.4.17.10) (Carboxypeptidase H) (CPH) |
| | (Enkephalin convertase) (Prohormone-processing carboxypeptidase) |
| P14384 | Carboxypeptidase M (CPM) (EC 3.4.17.12) |
| P22792 | Carboxypeptidase N subunit 2 (Carboxypeptidase N 83 kDa chain) |
| | (Carboxypeptidase N large subunit) (Carboxypeptidase N polypeptide 2) |
| | (Carboxypeptidase N regulatory subunit) |
| Q9Y646 | Carboxypeptidase Q (EC 3.4.17.—) (Lysosomal dipeptidase) (Plasma glutamate |
| | carboxypeptidase) |
| Q9H3G5 | Probable serine carboxypeptidase CPVL (EC 3.4.16.—) (Carboxypeptidase, |
| | vitellogenic-like) (Vitellogenic carboxypeptidase-like protein) (VCP-like |
| | protein) (hVLP) |
| Q66K79 | Carboxypeptidase Z (CPZ) (EC 3.4.17.—) |
| O75629 | Protein CREG1 (Cellular repressor of E1 A-stimulated genes 1) |
| P24387 | Corticotropin-releasing factor-binding protein (CRF-BP) (CRF-binding protein) |
| | (Corticotropin-releasing hormone-binding protein) (CRH-BP) |
| P34998 | Corticotropin-releasing factor receptor 1 (CRF-R-1) (CRF-R1) (CRFR-1) |
| | (Corticotropin-releasing hormone receptor 1) (CRH-R-1) (CRH-R1) |
| P46108 | Adapter molecule crk (Proto-oncogene c-Crk) (p38) |
| O75462 | Cytokine receptor-like factor 1 (Cytokine-like factor 1) (CLF-1) (ZcytoR5) |
| Q14894 | Ketimine reductase mu-crystallin (EC 1.5.1.25) (NADP-regulated thyroid- |
| | hormone-binding protein) |
| O75390 | Citrate synthase, mitochondrial (EC 2.3.3.1) (Citrate (Si)-synthase) |
| P07333 | Macrophage colony-stimulating factor 1 receptor (CSF-1 receptor) (CSF-1-R) |
| | (CSF-1R) (M-CSF-R) (EC 2.7.10.1) (Proto-oncogene c-Fms) (CD antigen |
| | CD115) |
| Q96PZ7 | CUB and sushi domain-containing protein 1 (CUB and sushi multiple domains |
| | protein 1) |
| Q7Z408 | CUB and sushi domain-containing protein 2 (CUB and sushi multiple domains |
| | protein 2) |
| Q6UVK1 | Chondroitin sulfate proteoglycan 4 (Chondroitin sulfate proteoglycan NG2) |
| | (Melanoma chondroitin sulfate proteoglycan) (Melanoma-associated chondroitin |
| | sulfate proteoglycan) |
| O95196 | Chondroitin sulfate proteoglycan 5 (Acidic leucine-rich EGF-like domain- |
| | containing brain protein) (Neuroglycan C) |
| Q16527 | Cysteine and glycine-rich protein 2 (Cysteine-rich protein 2) (CRP2) (LIM |
| | domain only protein 5) (LMO-5) (Smooth muscle cell LIM protein) (SmLIM) |
| Q01459 | Di-N-acetylchitobiase (EC 3.2.1.—) |
| P10619 | Lysosomal protective protein (EC 3.4.16.5) (Carboxypeptidase C) |
| | (Carboxypeptidase L) (Cathepsin A) (Protective protein cathepsin A) (PPCA) |
| | (Protective protein for beta-galactosidase) [Cleaved into: Lysosomal protective |
| | protein 32 kDa chain; Lysosomal protective protein 20 kDa chain] |
| P53634 | Dipeptidyl peptidase 1 (EC 3.4.14.1) (Cathepsin C) (Cathepsin J) (Dipeptidyl |
| | peptidase I) (DPP-I) (DPPI) (Dipeptidyl transferase) [Cleaved into: Dipeptidyl |
| | peptidase 1 exclusion domain chain (Dipeptidyl peptidase I exclusion domain |
| | chain); Dipeptidyl peptidase 1 heavy chain (Dipeptidyl peptidase I heavy chain); |
| | Dipeptidyl peptidase 1 light chain (Dipeptidyl peptidase I light chain)] |
| P07339 | Cathepsin D (EC 3.4.23.5) [Cleaved into: Cathepsin D light chain; Cathepsin D |
| | heavy chain] |
| Q9UBX1 | Cathepsin F (CATSF) (EC 3.4.22.41) |
| P09668 | Pro-cathepsin H [Cleaved into: Cathepsin H mini chain; Cathepsin H (EC |
| | 3.4.22.16); Cathepsin H heavy chain; Cathepsin H light chain] |
| P07711 | Cathepsin L1 (EC 3.4.22.15) (Cathepsin L) (Major excreted protein) (MEP) |
| | [Cleaved into: Cathepsin L1 heavy chain; Cathepsin L1 light chain] |
| P78310 | Coxsackievirus and adenovirus receptor (CAR) (hCAR) (CVB3-binding protein) |
| | (Coxsackievirus B-adenovirus receptor) (HCVADR) |
| P04839 | Cytochrome b-245 heavy chain (EC 1.—.—.—) (CGD91-phox) (Cytochrome b(558) |
| | subunit beta) (Cytochrome b558 subunit beta) (Heme-binding membrane |
| | glycoprotein gp91phox) (NADPH oxidase 2) (Neutrophil cytochrome b 91 kDa |
| | polypeptide) (Superoxide-generating NADPH oxidase heavy chain subunit) |
| | (gp91-1) (gp91-phox) (p22 phagocyte B-cytochrome) |
| Q14118 | Dystroglycan (Dystrophin-associated glycoprotein 1) [Cleaved into: Alpha- |
| | dystroglycan (Alpha-DG); Beta-dystroglycan (Beta-DG)] |
| Q96JQ0 | Protocadherin-16 (Cadherin-19) (Cadherin-25) (Fibroblast cadherin-1) (Protein |
| | dachsous homolog 1) |
| P07585 | Decorin (Bone proteoglycan II) (PG-S2) (PG40) |
| Q6IQ26 | DENN domain-containing protein 5A (Rab6-interacting protein 1) (Rab6IP1) |
| O75140 | GATOR complex protein DEPDC5 (DEP domain-containing protein 5) |
| P98153 | Integral membrane protein DGCR2/IDD |
| P49619 | Diacylglycerol kinase gamma (DAG kinase gamma) (EC 2.7.1.107) |
| | (Diglyceride kinase gamma) (DGK-gamma) |
| Q9UBP4 | Dickkopf-related protein 3 (Dickkopf-3) (Dkk-3) (hDkk-3) |
| Q92796 | Disks large homolog 3 (Neuroendocrine-DLG) (Synapse-associated protein 102) |
| | (SAP-102) (SAP102) (XLMR) |
| O00115 | Deoxyribonuclease-2-alpha (EC 3.1.22.1) (Acid DNase) (Deoxyribonuclease II |
| | alpha) (DNase II alpha) (Lysosomal DNase II) (R31240_2) |
| Q8NFT8 | Delta and Notch-like epidermal growth factor-related receptor |
| Q05193 | Dynamin-1 (EC 3.6.5.5) |
| P26358 | DNA (cytosine-5)-methyltransferase 1 (Dnmt1) (EC 2.1.1.37) (CXXC-type zinc |
| | finger protein 9) (DNA methyltransferase HsaI) (DNA MTase HsaI) (M.HsaI) |
| | (MCMT) |
| Q8IZD9 | Dedicator of cytokinesis protein 3 (Modifier of cell adhesion) (Presenilin- |
| | binding protein) (PBP) |
| Q8N608 | Inactive dipeptidyl peptidase 10 (Dipeptidyl peptidase IV-related protein 3) |
| | (DPRP-3) (Dipeptidyl peptidase X) (DPP X) (Dipeptidyl peptidase-like protein |
| | 2) (DPL2) |
| P42658 | Dipeptidyl aminopeptidase-like protein 6 (DPPX) (Dipeptidyl aminopeptidase- |
| | related protein) (Dipeptidyl peptidase 6) (Dipeptidyl peptidase IV-like protein) |
| | (Dipeptidyl peptidase VI) (DPP VI) |
| Q9UHL4 | Dipeptidyl peptidase 2 (EC 3.4.14.2) (Dipeptidyl aminopeptidase II) (Dipeptidyl |
| | peptidase 7) (Dipeptidyl peptidase II) (DPP II) (Quiescent cell proline |
| | dipeptidase) |
| Q16555 | Dihydropyrimidinase-related protein 2 (DRP-2) (Collapsin response mediator |
| | protein 2) (CRMP-2) (N2A3) (Unc-33-like phosphoprotein 2) (ULIP-2) |
| Q14195 | Dihydropyrimidinase-related protein 3 (DRP-3) (Collapsin response mediator |
| | protein 4) (CRMP-4) (Unc-33-like phosphoprotein 1) (ULIP-1) |
| P21728 | D(1A) dopamine receptor (Dopamine D1 receptor) |
| Q02487 | Desmocollin-2 (Cadherin family member 2) (Desmocollin-3) (Desmosomal |
| | glycoprotein II) (Desmosomal glycoprotein III) |
| Q14574 | Desmocollin-3 (Cadherin family member 3) (Desmocollin-4) (HT-CP) |
| Q14126 | Desmoglein-2 (Cadherin family member 5) (HDGC) |
| P42892 | Endothelin-converting enzyme 1 (ECE-1) (EC 3.4.24.71) |
| Q15075 | Early endosome antigen 1 (Endosome-associated protein p162) (Zinc finger |
| | FYVE domain-containing protein 2) |
| P68104 | Elongation factor 1-alpha 1 (EF-1-alpha-1) (Elongation factor Tu) (EF-Tu) |
| | (Eukaryotic elongation factor 1 A-1) (eEF1A-1) (Leukocyte receptor cluster |
| | member 7) |
| Q05639 | Elongation factor 1-alpha 2 (EF-1-alpha-2) (Eukaryotic elongation factor 1 A-2) |
| | (eEF1A-2) (Stati?1) |
| Q12805 | EGF-containing fibulin-like extracellular matrix protein 1 (Extracellular protein |
| | S1-5) (Fibrillin-like protein) (Fibulin-3) (FIBL-3) |
| P52797 | Ephrin-A3 (EFL-2) (EHK1 ligand) (EHK1-L) (EPH-related receptor tyrosine |
| | kinase ligand 3) (LERK-3) |
| P52803 | Ephrin-A5 (AL-1) (EPH-related receptor tyrosine kinase ligand 7) (LERK-7) |
| P98172 | Ephrin-B1 (EFL-3) (ELK ligand) (ELK-L) (EPH-related receptor tyrosine |
| | kinase ligand 2) (LERK-2) |
| P52799 | Ephrin-B2 (EPH-related receptor tyrosine kinase ligand 5) (LERK-5) (HTK |
| | ligand) (HTK-L) |
| Q15768 | Ephrin-B3 (EPH-related receptor transmembrane ligand ELK-L3) (EPH-related |
| | receptor tyrosine kinase ligand 8) (LERK-8) |
| P00533 | Epidermal growth factor receptor (EC 2.7.10.1) (Proto-oncogene c-ErbB-1) |
| | (Receptor tyrosine-protein kinase erbB-1) |
| P08246 | Neutrophil elastase (EC 3.4.21.37) (Bone marrow serine protease) (Elastase-2) |
| | (Human leukocyte elastase) (HLE) (Medullasin) (PMN elastase) |
| P0C7U0 | Protein ELFN1 (Extracellular leucine-rich repeat and fibronectin type-III |
| | domain-containing protein 1) (Protein phosphatase 1 regulatory subunit 28) |
| Q5R3F8 | Protein phosphatase 1 regulatory subunit 29 (Extracellular leucine-rich repeat |
| | and fibronectin type III domain-containing protein 2) (Leucine-rich repeat and |
| | fibronectin type-III domain-containing protein 6) (Leucine-rich repeat- |
| | containing protein 62) |
| Q96BJ8 | Engulfment and cell motility protein 3 |
| Q6PCB8 | Embigin |
| Q8N766 | ER membrane protein complex subunit 1 |
| Q5UCC4 | ER membrane protein complex subunit 10 (Hematopoietic signal peptide- |
| | containing membrane domain-containing protein 1) |
| Q9Y6C2 | EMILIN-1 (Elastin microfibril interface-located protein 1) (Elastin microfibril |
| | interfacer 1) |
| Q9BXX0 | EMILIN-2 (Elastin microfibril interface-located protein 2) (Elastin microfibril |
| | interfacer 2) (Protein FOAP-10) |
| P54849 | Epithelial membrane protein 1 (EMP-1) (CL-20) (Protein B4B) (Tumor- |
| | associated membrane protein) |
| P54852 | Epithelial membrane protein 3 (EMP-3) (Hematopoietic neural membrane |
| | protein 1) (HNMP-1) (Protein YMP) |
| O94919 | Endonuclease domain-containing 1 protein (EC 3.1.30.—) |
| P09104 | Gamma-enolase (EC 4.2.1.11) (2-phospho-D-glycerate hydro-lyase) (Enolase 2) |
| | (Neural enolase) (Neuron-specific enolase) (NSE) |
| Q07075 | Glutamyl aminopeptidase (EAP) (EC 3.4.11.7) (Aminopeptidase A) (AP-A) |
| | (Differentiation antigen gp160) (CD antigen CD249) |
| P22413 | Ectonucleotide pyrophosphatase/phosphodiesterase family member 1 (E-NPP 1) |
| | (Membrane component chromosome 6 surface marker 1) (Phosphodiesterase |
| | Enucleotide pyrophosphatase 1) (Plasma-cell membrane glycoprotein PC-1) |
| | [Includes: Alkaline phosphodiesterase I (EC 3.1.4.1); Nucleotide |
| | pyrophosphatase (NPPase) (EC 3.6.1.9) (Nucleotide diphosphatase)] |
| Q13822 | Ectonucleotide pyrophosphatase/phosphodiesterase family member 2 (E-NPP 2) |
| | (EC 3.1.4.39) (Autotaxin) (Extracellular lysophospholipase D) (LysoPLD) |
| Q9Y6X5 | Bis(5′-adenosyl)-triphosphatase ENPP4 (EC 3.6.1.29) (AP3A hydrolase) |
| | (AP3Aase) (Ectonucleotide pyrophosphatase/phosphodiesterase family member |
| | 4) (E-NPP 4) (NPP-4) |
| Q9UJA9 | Ectonucleotide pyrophosphatase/phosphodiesterase family member 5 (E-NPP 5) |
| | (NPP-5) (EC 3.1.—.—) |
| Q6UWR7 | Ectonucleotide pyrophosphatase/phosphodiesterase family member 6 (E-NPP 6) |
| | (NPP-6) (EC 3.1.4.—) (EC 3.1.4.38) (Choline-specific glycerophosphodiester |
| | phosphodiesterase) (Glycerophosphocholine cholinephosphodiesterase) (GPC- |
| | Cpde) |
| P49961 | Ectonucleoside triphosphate diphosphohydrolase 1 (NTPDase 1) (EC 3.6.1.5) |
| | (Ecto-ATP diphosphohydrolase 1) (Ecto-ATPDase 1) (Ecto-ATPase 1) (Ecto- |
| | apyrase) (Lymphoid cell activation antigen) (CD antigen CD39) |
| Q9Y5L3 | Ectonucleoside triphosphate diphosphohydrolase 2 (NTPDase 2) (EC 3.6.1.—) |
| | (CD39 antigen-like 1) (Ecto-ATP diphosphohydrolase 2) (Ecto-ATPDase 2) |
| | (Ecto-ATPase 2) |
| O75355 | Ectonucleoside triphosphate diphosphohydrolase 3 (NTPDase 3) (EC 3.6.1.5) |
| | (CD39 antigen-like 3) (Ecto-ATP diphosphohydrolase 3) (Ecto-ATPDase 3) |
| | (Ecto-ATPase 3) (Ecto-apyrase 3) (HB6) |
| Q5NDL2 | EGF domain-specific O-linked N-acetylglucosamine transferase (EC 2.4.1.255) |
| | (Extracellular O-linked N-acetylglucosamine transferase) |
| Q9UM22 | Mammalian ependymin-related protein 1 (MERP-1) (Upregulated in colorectal |
| | cancer gene 1 protein) |
| Q9HCE0 | Ectopic P granules protein 5 homolog |
| P54764 | Ephrin type-A receptor 4 (EC 2.7.10.1) (EPH-like kinase 8) (EK8) (hEK8) |
| | (Tyrosine-protein kinase TYRO1) (Tyrosine-protein kinase receptor SEK) |
| P54756 | Ephrin type-A receptor 5 (EC 2.7.10.1) (Brain-specific kinase) (EPH homology |
| | kinase 1) (EHK-1) (EPH-like kinase 7) (EK7) (hEK7) |
| P29323 | Ephrin type-B receptor 2 (EC 2.7.10.1) (Developmentally-regulated Eph-related |
| | tyrosine kinase) (ELK-related tyrosine kinase) (EPH tyrosine kinase 3) (EPH- |
| | like kinase 5) (EK5) (hEK5) (Renal carcinoma antigen NY-REN-47) (Tyrosine- |
| | protein kinase TYRO5) (Tyrosine-protein kinase receptor EPH-3) |
| P54760 | Ephrin type-B receptor 4 (EC 2.7.10.1) (Hepatoma transmembrane kinase) |
| | (Tyrosine-protein kinase TYRO11) |
| P07814 | Bifunctional glutamate/proline--tRNA ligase (Bifunctional aminoacyl-tRNA |
| | synthetase) (Cell proliferation-inducing gene 32 protein) (Glutamatyl-prolyl- |
| | tRNA synthetase) [Includes: Glutamate--tRNA ligase (EC 6.1.1.17) (Glutamyl- |
| P25445 | Tumor necrosis factor receptor superfamily member 6 (Apo-1 antigen) |
| | (Apoptosis-mediating surface antigen FAS) (FASLG receptor) (CD antigen |
| | CD95) |
| P49327 | Fatty acid synthase (EC 2.3.1.85) [Includes: [Acyl-carrier-protein] S- |
| | acetyltransferase (EC 2.3.1.38); [Acyl-carrier-protein] S-malonyltransferase (EC |
| | 2.3.1.39); 3-oxoacyl-[acyl-carrier-protein] synthase (EC 2.3.1.41); 3-oxoacyl- |
| | [acyl-carrier-protein] reductase (EC 1.1.1.100); 3-hydroxyacyl-[acyl-carrier- |
| | protein] dehydratase (EC 4.2.1.59); Enoyl-[acyl-carrier-protein] reductase (EC |
| | 1.3.1.39); Oleoyl-[acyl-carrier-protein] hydrolase (EC 3.1.2.14)] |
| Q14517 | Protocadherin Fat 1 (Cadherin family member 7) (Cadherin-related tumor |
| | suppressor homolog) (Protein fat homolog) [Cleaved into: Protocadherin Fat 1, |
| | nuclear form] |
| Q8TDW7 | Protocadherin Fat 3 (hFat3) (Cadherin family member 15) (FAT tumor |
| | suppressor homolog 3) |
| P23142 | Fibulin-1 (FIBL-1) |
| P98095 | Fibulin-2 (FIBL-2) |
| Q9UBX5 | Fibulin-5 (FIBL-5) (Developmental arteries and neural crest EGF-like protein) |
| | (Dance) (Urine p50 protein) (UP50) |
| Q53RD9 | Fibulin-7 (FIBL-7) |
| P35555 | Fibrillin-1 [Cleaved into: Asprosin] |
| Q9Y6R7 | IgGFc-binding protein (Fcgamma-binding protein antigen) (FcgammaBP) |
| P12314 | High affinity immunoglobulin gamma Fc receptor I (IgG Fc receptor I) (Fc- |
| | gamma RI) (FcRI) (Fc-gamma RIA) (FcgammaRIa) (CD antigen CD64) |
| P55899 | IgG receptor FcRn large subunit p51 (FcRn) (IgG Fc fragment receptor |
| | transporter alpha chain) (Neonatal Fc receptor) |
| P02671 | Fibrinogen alpha chain [Cleaved into: Fibrinopeptide A; Fibrinogen alpha chain] |
| P11362 | Fibroblast growth factor receptor 1 (FGFR-1) (EC 2.7.10.1) (Basic fibroblast |
| | growth factor receptor 1) (BFGFR) (bFGF-R-1) (Fms-like tyrosine kinase 2) |
| | (FLT-2) (N-sam) (Proto-oncogene c-Fgr) (CD antigen CD331) |
| P21802 | Fibroblast growth factor receptor 2 (FGFR-2) (EC 2.7.10.1) (K-sam) (KGFR) |
| | (Keratinocyte growth factor receptor) (CD antigen CD332) |
| P22607 | Fibroblast growth factor receptor 3 (FGFR-3) (EC 2.7.10.1) (CD antigen |
| | CD333) |
| Q14314 | Fibroleukin (Fibrinogen-like protein 2) (pT49) |
| P07954 | Fumarate hydratase, mitochondrial (Fumarase) (EC 4.2.1.2) |
| Q96AY3 | Peptidyl-prolyl cis-trans isomerase FKBP10 (PPIase FKBP10) (EC 5.2.1.8) (65 |
| | kDa FK506-binding protein) (65 kDa FKBP) (FKBP-65) (FK506-binding |
| | protein 10) (FKBP-10) (Immunophilin FKBP65) (Rotamase) |
| O95302 | Peptidyl-prolyl cis-trans isomerase FKBP9 (PPIase FKBP9) (EC 5.2.1.8) (63 |
| | kDa FK506-binding protein) (63 kDa FKBP) (FKBP-63) (FK506-binding |
| | protein 9) (FKBP-9) (Rotamase) |
| P21333 | Filamin-A (FLN-A) (Actin-binding protein 280) (ABP-280) (Alpha-filamin) |
| | (Endothelial actin-binding protein) (Filamin-1) (Non-muscle filamin) |
| Q9NZU1 | Leucine-rich repeat transmembrane protein FLRT1 (Fibronectin-like domain- |
| | containing leucine-rich transmembrane protein 1) |
| P17948 | Vascular endothelial growth factor receptor 1 (VEGFR-1) (EC 2.7.10.1) (Fms- |
| | like tyrosine kinase 1) (FLT-1) (Tyrosine-protein kinase FRT) (Tyrosine-protein |
| | kinase receptor FLT) (FLT) (Vascular permeability factor receptor) |
| Q06828 | Fibromodulin (FM) (Collagen-binding 59 kDa protein) (Keratan sulfate |
| | proteoglycan fibromodulin) (KSPG fibromodulin) |
| P02751 | Fibronectin (FN) (Cold-insoluble globulin) (CIG) [Cleaved into: Anastellin; |
| | Ugl-Y1; Ugl-Y2; Ugl-Y3] |
| Q04609 | Glutamate carboxypeptidase 2 (EC 3.4.17.21) (Cell growth-inhibiting gene 27 |
| | protein) (Folate hydrolase 1) (Folylpoly-gamma-glutamate carboxypeptidase) |
| | (FGCP) (Glutamate carboxypeptidase II) (GCPII) (Membrane glutamate |
| | carboxypeptidase) (mGCP) (N-acetylated-alpha-linked acidic dipeptidase I) |
| | (NAALADase I) (Prostate-specific membrane antigen) (PSM) (PSMA) |
| | (Pteroylpoly-gamma-glutamate carboxypeptidase) |
| Q9HBA9 | Putative N-acetylated-alpha-linked acidic dipeptidase (NAALADase) (EC 3.4.—.—) |
| | (Cell growth-inhibiting gene 26 protein) (Prostate-specific membrane antigen- |
| | like protein) (Putative folate hydrolase 1B) |
| P15328 | Folate receptor alpha (FR-alpha) (Adult folate-binding protein) (FBP) (Folate |
| | receptor 1) (Folate receptor, adult) (KB cells FBP) (Ovarian tumor-associated |
| | antigen MOv18) |
| P14207 | Folate receptor beta (FR-beta) (Folate receptor 2) (Folate receptor, |
| | fetal/placental) (Placental folate-binding protein) (FBP) |
| Q8IWF2 | FAD-dependent oxidoreductase domain-containing protein 2 (Endoplasmic |
| | reticulum flavoprotein associated with degradation) |
| Q12841 | Follistatin-related protein 1 (Follistatin-like protein 1) |
| Q6MZW2 | Follistatin-related protein 4 (Follistatin-like protein 4) |
| P02794 | Ferritin heavy chain (Ferritin H subunit) (EC 1.16.3.1) (Cell proliferation- |
| | inducing gene 15 protein) [Cleaved into: Ferritin heavy chain, N-terminally |
| | processed] |
| P04066 | Tissue alpha-L-fucosidase (EC 3.2.1.51) (Alpha-L-fucosidase I) (Alpha-L- |
| | fucoside fucohydrolase 1) (Alpha-L-fucosidase 1) |
| Q9BTY2 | Plasma alpha-L-fucosidase (EC 3.2.1.51) (Alpha-L-fucoside fucohydrolase 2) |
| | (Alpha-L-fucosidase 2) |
| P35637 | RNA-binding protein FUS (75 kDa DNA-pairing protein) (Oncogene FUS) |
| | (Oncogene TLS) (POMp75) (Translocated in liposarcoma protein) |
| Q9NPG1 | Frizzled-3 (Fz-3) (hFz3) |
| Q13283 | Ras GTPase-activating protein-binding protein 1 (G3BP-1) (EC 3.6.4.12) (EC |
| | 3.6.4.13) (ATP-dependent DNA helicase VIII) (hDH VIII) (GAP SH3 domain- |
| | binding protein 1) |
| Q9UN86 | Ras GTPase-activating protein-binding protein 2 (G3BP-2) (GAP SH3 domain- |
| | binding protein 2) |
| P10253 | Lysosomal alpha-glucosidase (EC 3.2.1.20) (Acid maltase) (Aglucosidase alfa) |
| | [Cleaved into: 76 kDa lysosomal alpha-glucosidase; 70 kDa lysosomal alpha- |
| | glucosidase] |
| Q9UBS5 | Gamma-aminobutyric acid type B receptor subunit 1 (GABA-B receptor 1) |
| | (GABA-B-R1) (GABA-BR1) (GABABR1) (Gb1) |
| O75899 | Gamma-aminobutyric acid type B receptor subunit 2 (GABA-B receptor 2) |
| | (GABA-B-R2) (GABA-BR2) (GABABR2) (Gb2) (G-protein coupled receptor |
| | 51) (HG20) |
| P47869 | Gamma-aminobutyric acid receptor subunit alpha-2 (GABA(A) receptor subunit |
| | alpha-2) |
| P34903 | Gamma-aminobutyric acid receptor subunit alpha-3 (GABA(A) receptor subunit |
| | alpha-3) |
| P48169 | Gamma-aminobutyric acid receptor subunit alpha-4 (GABA(A) receptor subunit |
| | alpha-4) |
| P31644 | Gamma-aminobutyric acid receptor subunit alpha-5 (GABA(A) receptor subunit |
| | alpha-5) |
| P18505 | Gamma-aminobutyric acid receptor subunit beta-1 (GABA(A) receptor subunit |
| | beta-1) |
| P47870 | Gamma-aminobutyric acid receptor subunit beta-2 (GABA(A) receptor subunit |
| | beta-2) |
| P28472 | Gamma-aminobutyric acid receptor subunit beta-3 (GABA(A) receptor subunit |
| | beta-3) |
| O14764 | Gamma-aminobutyric acid receptor subunit delta (GABA(A) receptor subunit |
| | delta) |
| P54803 | Galactocerebrosidase (GALCERase) (EC 3.2.1.46) (Galactocerebroside beta- |
| | galactosidase) (Galactosylceramidase) (Galactosylceramide beta-galactosidase) |
| P34059 | N-acetylgalactosamine-6-sulfatase (EC 3.1.6.4) (Chondroitinsulfatase) |
| | (Chondroitinase) (Galactose-6-sulfate sulfatase) (GalN6S) (N- |
| | acetylgalactosamine-6-sulfate sulfatase) (GalNAc6S sulfatase) |
| Q6IS24 | Polypeptide N-acetylgalactosaminyltransferase 17 (EC 2.4.1.41) (Polypeptide |
| | GalNAc transferase-like protein 3) (GalNAc-T-like protein 3) (pp-GaNTase-like |
| | protein 3) (Protein-UDP acetylgalactosaminyltransferase-like protein 3) (UDP- |
| | GalNAc: polypeptide N-acetylgalactosaminyltransferase-like protein 3) |
| | (Williams-Beuren syndrome chromosomal region 17 protein) |
| Q9HCQ5 | Polypeptide N-acetylgalactosaminyltransferase 9 (EC 2.4.1.41) (Polypeptide |
| | GalNAc transferase 9) (GalNAc-T9) (pp-GaNTase 9) (Protein-UDP |
| | acetylgalactosaminyltransferase 9) (UDP-GalNAc: polypeptide N- |
| | acetylgalactosaminyltransferase 9) |
| P04062 | Glucosylceramidase (EC 3.2.1.45) (Acid beta-glucosidase) (Alglucerase) (Beta- |
| | glucocerebrosidase) (Beta-GC) (D-glucosyl-N-acylsphingosine glucohydrolase) |
| | (Imiglucerase) |
| Q9Y2T3 | Guanine deaminase (Guanase) (Guanine aminase) (EC 3.5.4.3) (Guanine |
| | aminohydrolase) (GAH) (p51-nedasin) |
| Q9NZC3 | Glycerophosphodiester phosphodiesterase 1 (EC 3.1.4.—) (EC 3.1.4.44) |
| | (Membrane-interacting protein of RGS16) (RGS16-interacting membrane |
| | protein) |
| Q8WTR4 | Glycerophosphodiester phosphodiesterase domain-containing protein 5 (EC 3.1.—.—) |
| | (Glycerophosphodiester phosphodiesterase 2) |
| P56159 | GDNF family receptor alpha-1 (GDNF receptor alpha-1) (GDNFR-alpha-1) |
| | (GFR-alpha-1) (RET ligand 1) (TGF-beta-related neurotrophic factor receptor 1) |
| O00451 | GDNF family receptor alpha-2 (GDNF receptor alpha-2) (GDNFR-alpha-2) |
| | (GFR-alpha-2) (GDNF receptor beta) (GDNFR-beta) (Neurturin receptor alpha) |
| | (NRTNR-alpha) (NTNR-alpha) (RET ligand 2) (TGF-beta-related neurotrophic |
| | factor receptor 2) |
| P38435 | Vitamin K-dependent gamma-carboxylase (EC 4.1.1.90) (Gamma-glutamyl |
| | carboxylase) (Peptidyl-glutamate 4-carboxylase) (Vitamin K gamma glutamyl |
| | carboxylase) |
| P19440 | Glutathione hydrolase 1 proenzyme (EC 3.4.19.13) (Gamma- |
| | glutamyltransferase 1) (Gamma-glutamyltranspeptidase 1) (GGT 1) (EC 2.3.2.2) |
| | (Leukotriene-C4 hydrolase) (EC 3.4.19.14) (CD antigen CD224) [Cleaved into: |
| | Glutathione hydrolase 1 heavy chain; Glutathione hydrolase 1 light chain] |
| Q9UJ14 | Glutathione hydrolase 7 (EC 3.4.19.13) (Gamma-glutamyltransferase 7) (GGT |
| | 7) (EC 2.3.2.2) (Gamma-glutamyltransferase-like 3) (Gamma- |
| | glutamyltransferase-like 5) (Gamma-glutamyltranspeptidase 7) [Cleaved into: |
| | Glutathione hydrolase 7 heavy chain; Glutathione hydrolase 7 light chain] |
| Q14390 | Glutathione hydrolase light chain 2 (Gamma-glutamyltransferase light chain 2) |
| | (Gamma-glutamyltransferase-like protein 4) |
| P16278 | Beta-galactosidase (EC 3.2.1.23) (Acid beta-galactosidase) (Lactase) (Elastin |
| | receptor 1) |
| Q92896 | Golgi apparatus protein 1 (CFR-1) (Cysteine-rich fibroblast growth factor |
| | receptor) (E-selectin ligand 1) (ESL-1) (Golgi sialoglycoprotein MG-160) |
| Q8WWB7 | Glycosylated lysosomal membrane protein (Lysosomal protein NCU-G1) |
| P48167 | Glycine receptor subunit beta (Glycine receptor 58 kDa subunit) |
| O94925 | Glutaminase kidney isoform, mitochondrial (GLS) (EC 3.5.1.2) (K-glutaminase) |
| | (L-glutamine amidohydrolase) |
| P62873 | Guanine nucleotide-binding protein G(I)/G(S)/G(T) subunit beta-1 (Transducin |
| | beta chain 1) |
| P62879 | Guanine nucleotide-binding protein G(I)/G(S)/G(T) subunit beta-2 (G protein |
| | subunit beta-2) (Transducin beta chain 2) |
| Q3T906 | N-acetylglucosamine-1-phosphotransferase subunits alpha/beta (EC 2.7.8.17) |
| | (GlcNAc-1-phosphotransferase subunits alpha/beta) (Stealth protein GNPTAB) |
| | (UDP-N-acetylglucosamine-1-phosphotransferase subunits alpha/beta) [Cleaved |
| | into: N-acetylglucosamine-1-phosphotransferase subunit alpha; N- |
| | acetylglucosamine-1-phosphotransferase subunit beta] |
| P15586 | N-acetylglucosamine-6-sulfatase (EC 3.1.6.14) (Glucosamine-6-sulfatase) (G6S) |
| Q13439 | Golgin subfamily A member 4 (256 kDa golgin) (Golgin-245) (Protein 72.1) |
| | (Trans-Golgi p230) |
| O00461 | Golgi integral membrane protein 4 (Golgi integral membrane protein, cis) |
| | (GIMPc) (Golgi phosphoprotein 4) (Golgi-localized phosphoprotein of 130 kDa) |
| | (Golgi phosphoprotein of 130 kDa) |
| Q8NBJ4 | Golgi membrane protein 1 (Golgi membrane protein GP73) (Golgi |
| | phosphoprotein 2) |
| P00505 | Aspartate aminotransferase, mitochondrial (mAspAT) (EC 2.6.1.1) (EC 2.6.1.7) |
| | (Fatty acid-binding protein) (FABP-1) (Glutamate oxaloacetate transaminase 2) |
| | (Kynurenine aminotransferase 4) (Kynurenine aminotransferase IV) |
| | (Kynurenine--oxoglutarate transaminase 4) (Kynurenine--oxoglutarate |
| | transaminase IV) (Plasma membrane-associated fatty acid-binding protein) |
| | (FABPpm) (Transaminase A) |
| P35052 | Glypican-1 [Cleaved into: Secreted glypican-1] |
| P51674 | Neuronal membrane glycoprotein M6-a (M6a) |
| Q13491 | Neuronal membrane glycoprotein M6-b (M6b) |
| Q14956 | Transmembrane glycoprotein NMB (Transmembrane glycoprotein HGFIN) |
| Q5VW38 | Protein GPR107 (Lung seven transmembrane receptor 1) |
| Q7Z3F1 | Integral membrane protein GPR155 (G-protein coupled receptor PGR22) |
| Q5T848 | Probable G-protein coupled receptor 158 |
| Q8IYS0 | GRAM domain-containing protein 1C |
| P42261 | Glutamate receptor 1 (GluR-1) (AMPA-selective glutamate receptor 1) (GluR- |
| | A) (GluR-K1) (Glutamate receptor ionotropic, AMPA 1) (GluA1) |
| P42262 | Glutamate receptor 2 (GluR-2) (AMPA-selective glutamate receptor 2) (GluR- |
| | B) (GluR-K2) (Glutamate receptor ionotropic, AMPA 2) (GluA2) |
| P42263 | Glutamate receptor 3 (GluR-3) (AMPA-selective glutamate receptor 3) (GluR- |
| | C) (GluR-K3) (Glutamate receptor ionotropic, AMPA 3) (GluA3) |
| P48058 | Glutamate receptor 4 (GluR-4) (GluR4) (AMPA-selective glutamate receptor 4) |
| | (GluR-D) (Glutamate receptor ionotropic, AMPA 4) (GluA4) |
| Q9ULK0 | Glutamate receptor ionotropic, delta-1 (GluD1) (GluR delta-1 subunit) |
| O43424 | Glutamate receptor ionotropic, delta-2 (GluD2) (GluR delta-2 subunit) |
| Q13002 | Glutamate receptor ionotropic, kainate 2 (GluK2) (Excitatory amino acid |
| | receptor 4) (EAA4) (Glutamate receptor 6) (GluR-6) (GluR6) |
| Q13003 | Glutamate receptor ionotropic, kainate 3 (GluK3) (Excitatory amino acid |
| | receptor 5) (EAA5) (Glutamate receptor 7) (GluR-7) (GluR7) |
| Q16478 | Glutamate receptor ionotropic, kainate 5 (GluK5) (Excitatory amino acid |
| | receptor 2) (EAA2) (Glutamate receptor KA-2) (KA2) |
| Q05586 | Glutamate receptor ionotropic, NMDA 1 (GluN1) (Glutamate [NMDA] receptor |
| | subunit zeta-1) (N-methyl-D-aspartate receptor subunit NR1) (NMD-R1) |
| Q12879 | Glutamate receptor ionotropic, NMDA 2A (GluN2A) (Glutamate [NMDA] |
| | receptor subunit epsilon-1) (N-methyl D-aspartate receptor subtype 2A) |
| | (NMDAR2A) (NR2A) (hNR2A) |
| Q13224 | Glutamate receptor ionotropic, NMDA 2B (GluN2B) (Glutamate [NMDA] |
| | receptor subunit epsilon-2) (N-methyl D-aspartate receptor subtype 2B) |
| | (NMDAR2B) (NR2B) (N-methyl-D-aspartate receptor subunit 3) (NR3) (hNR3) |
| Q13255 | Metabotropic glutamate receptor 1 (mGluR1) |
| Q14416 | Metabotropic glutamate receptor 2 (mGluR2) |
| Q14832 | Metabotropic glutamate receptor 3 (mGluR3) |
| P41594 | Metabotropic glutamate receptor 5 (mGluR5) |
| Q14831 | Metabotropic glutamate receptor 7 (mGluR7) |
| P25092 | Heat-stable enterotoxin receptor (STA receptor) (hSTAR) (EC 4.6.1.2) |
| | (Guanylyl cyclase C) (GC-C) (Intestinal guanylate cyclase) |
| Q4G148 | Glucoside xylosyltransferase 1 (EC 2.4.2.n2) (Glycosyltransferase 8 domain- |
| | containing protein 3) |
| P84243 | Histone H3.3 |
| Q16836 | Hydroxyacyl-coenzyme A dehydrogenase, mitochondrial (HCDH) (EC 1.1.1.35) |
| | (Medium and short-chain L-3-hydroxyacyl-coenzyme A dehydrogenase) (Short- |
| | chain 3-hydroxyacyl-CoA dehydrogenase) |
| Q86UW8 | Hyaluronan and proteoglycan link protein 4 (Brain link protein 2) |
| Q9NVX0 | HAUS augmin-like complex subunit 2 (Centrosomal protein of 27 kDa) (Cep27) |
| Q9ULT8 | E3 ubiquitin-protein ligase HECTD1 (EC 2.3.2.26) (E3 ligase for inhibin |
| | receptor) (EULIR) (HECT domain-containing protein 1) (HECT-type E3 |
| | ubiquitin transferase HECTD1) |
| Q9P2P5 | E3 ubiquitin-protein ligase HECW2 (EC 2.3.2.26) (HECT, C2 and WW domain- |
| | containing protein 2) (HECT-type E3 ubiquitin transferase HECW2) (NEDD4- |
| | like E3 ubiquitin-protein ligase 2) |
| Q14CZ8 | Hepatocyte cell adhesion molecule (Protein hepaCAM) |
| P14210 | Hepatocyte growth factor (Hepatopoietin-A) (Scatter factor) (SF) [Cleaved into: |
| | Hepatocyte growth factor alpha chain; Hepatocyte growth factor beta chain] |
| Q68CP4 | Heparan-alpha-glucosaminide N-acetyltransferase (EC 2.3.1.78) |
| | (Transmembrane protein 76) |
| Q96QV1 | Hedgehog-interacting protein (HHIP) (HIP) |
| P68431 | Histone H3.1 (Histone H3/a) (Histone H3/b) (Histone H3/c) (Histone H3/d) |
| | (Histone H3/f) (Histone H3/h) (Histone H3/i) (Histone H3/j) (Histone H3/k) |
| | (Histone H3/l) |
| Q71DI3 | Histone H3.2 (Histone H3/m) (Histone H3/o) |
| P19367 | Hexokinase-1 (EC 2.7.1.1) (Brain form hexokinase) (Hexokinase type I) (HK I) |
| P30459 | HLA class I histocompatibility antigen, A-74 alpha chain (Aw-19) (Aw-74) |
| | (MHC class I antigen A*74) |
| P13746 | HLA class I histocompatibility antigen, A-11 alpha chain (MHC class I antigen |
| | A*11) |
| P04439 | HLA class I histocompatibility antigen, A-3 alpha chain (MHC class I antigen |
| | A*3) |
| P10314 | HLA class I histocompatibility antigen, A-32 alpha chain (MHC class I antigen |
| | A*32) |
| P01891 | HLA class I histocompatibility antigen, A-68 alpha chain (Aw-68) (HLA class I |
| | histocompatibility antigen, A-28 alpha chain) (MHC class I antigen A*68) |
| P18464 | HLA class I histocompatibility antigen, B-51 alpha chain (MHC class I antigen |
| | B*51) |
| P01889 | HLA class I histocompatibility antigen, B-7 alpha chain (MHC class I antigen |
| | B*7) |
| Q29960 | HLA class I histocompatibility antigen, Cw-16 alpha chain (MHC class I antigen |
| | Cw*16) |
| Q07000 | HLA class I histocompatibility antigen, Cw-15 alpha chain (MHC class I antigen |
| | Cw*15) |
| P10321 | HLA class I histocompatibility antigen, Cw-7 alpha chain (MHC class I antigen |
| | Cw*7) |
| P20036 | HLA class II histocompatibility antigen, DP alpha 1 chain (DP(W3)) (DP(W4)) |
| | (HLA-SB alpha chain) (MHC class II DP3-alpha) (MHC class II DPA1) |
| P04440 | HLA class II histocompatibility antigen, DP beta 1 chain (HLA class II |
| | histocompatibility antigen, DP(W4) beta chain) (MHC class II antigen DPB1) |
| P01903 | HLA class II histocompatibility antigen, DR alpha chain (MHC class II antigen |
| | DRA) |
| P04229 | HLA class II histocompatibility antigen, DRB1-1 beta chain (MHC class II |
| | antigen DRB1*1) (DR-1) (DR1) |
| Q8NDA2 | Hemicentin-2 |
| P09429 | High mobility group protein B1 (High mobility group protein 1) (HMG-1) |
| P14866 | Heterogeneous nuclear ribonucleoprotein L (hnRNP L) |
| P00738 | Haptoglobin (Zonulin) [Cleaved into: Haptoglobin alpha chain; Haptoglobin |
| | beta chain] |
| Q5SSJ5 | Heterochromatin protein 1-binding protein 3 (Protein HP1-BP74) |
| P02790 | Hemopexin (Beta-1B-glycoprotein) |
| P04196 | Histidine-rich glycoprotein (Histidine-proline-rich glycoprotein) (HPRG) |
| Q7LGA3 | Heparan sulfate 2-O-sulfotransferase 1 (2-O-sulfotransferase) (2OST) (EC |
| | 2.8.2.—) |
| Q8IZP7 | Heparan-sulfate 6-O-sulfotransferase 3 (HS6ST-3) (EC 2.8.2.—) |
| P28845 | Corticosteroid 11-beta-dehydrogenase isozyme 1 (EC 1.1.1.146) (11-beta- |
| | hydroxysteroid dehydrogenase 1) (11-DH) (11-beta-HSD1) (Short chain |
| | dehydrogenase/reductase family 26C member 1) |
| P51659 | Peroxisomal multifunctional enzyme type 2 (MFE-2) (17-beta-hydroxysteroid |
| | dehydrogenase 4) (17-beta-HSD 4) (D-bifunctional protein) (DBP) |
| | (Multifunctional protein 2) (MPF-2) (Short chain dehydrogenase/reductase |
| | family 8C member 1) [Cleaved into: (3R)-hydroxyacyl-CoA dehydrogenase (EC |
| | 1.1.1.n12); Enoyl-CoA hydratase 2 (EC 4.2.1.107) (EC 4.2.1.119) (3-alpha,7- |
| | alpha,12-alpha-trihydroxy-5-beta-cholest-24-enoyl-CoA hydratase)] |
| P14625 | Endoplasmin (94 kDa glucose-regulated protein) (GRP-94) (Heat shock protein |
| | 90 kDa beta member 1) (Tumor rejection antigen 1) (gp96 homolog) |
| O43301 | Heat shock 70 kDa protein 12A |
| P48723 | Heat shock 70 kDa protein 13 (Microsomal stress-70 protein ATPase core) |
| | (Stress-70 protein chaperone microsome-associated 60 kDa protein) |
| P38646 | Stress-70 protein, mitochondrial (75 kDa glucose-regulated protein) (GRP-75) |
| | (Heat shock 70 kDa protein 9) (Mortalin) (MOT) (Peptide-binding protein 74) |
| | (PBP74) |
| P98160 | Basement membrane-specific heparan sulfate proteoglycan core protein (HSPG) |
| | (Perlecan) (PLC) [Cleaved into: Endorepellin; LG3 peptide] |
| Q9Y4L1 | Hypoxia up-regulated protein 1 (150 kDa oxygen-regulated protein) (ORP-150) |
| | (170 kDa glucose-regulated protein) (GRP-170) |
| P05362 | Intercellular adhesion molecule 1 (ICAM-1) (Major group rhinovirus receptor) |
| | (CD antigen CD54) |
| P13598 | Intercellular adhesion molecule 2 (ICAM-2) (CD antigen CD102) |
| Q9UMF0 | Intercellular adhesion molecule 5 (ICAM-5) (Telencephalin) |
| O75144 | ICOS ligand (B7 homolog 2) (B7-H2) (B7-like protein Gl50) (B7-related protein |
| | 1) (B7RP-1) (CD antigen CD275) |
| P22304 | Iduronate 2-sulfatase (EC 3.1.6.13) (Alpha-L-iduronate sulfate sulfatase) |
| | (Idursulfase) [Cleaved into: Iduronate 2-sulfatase 42 kDa chain; Iduronate 2- |
| | sulfatase 14 kDa chain] |
| P08069 | Insulin-like growth factor 1 receptor (EC 2.7.10.1) (Insulin-like growth factor I |
| | receptor) (IGF-I receptor) (CD antigen CD221) [Cleaved into: Insulin-like |
| | growth factor 1 receptor alpha chain; Insulin-like growth factor 1 receptor beta |
| | chain] |
| P11717 | Cation-independent mannose-6-phosphate receptor (CI Man-6-P receptor) (CI- |
| | MPR) (M6PR) (300 kDa mannose 6-phosphate receptor) (MPR 300) (Insulin- |
| | like growth factor 2 receptor) (Insulin-like growth factor II receptor) (IGF-II |
| | receptor) (M6P/IGF2 receptor) (M6P/IGF2R) (CD antigen CD222) |
| P01876 | Immunoglobulin heavy constant alpha 1 (Ig alpha-1 chain C region) (Ig alpha-1 |
| | chain C region BUR) (Ig alpha-1 chain C region TRO) |
| P01877 | Immunoglobulin heavy constant alpha 2 (Ig alpha-2 chain C region) (Ig alpha-2 |
| | chain C region BUT) (Ig alpha-2 chain C region LAN) |
| P01859 | Immunoglobulin heavy constant gamma 2 (Ig gamma-2 chain C region) (Ig |
| | gamma-2 chain C region DOT) (Ig gamma-2 chain C region TIL) (Ig gamma-2 |
| | chain C region ZIE) |
| P01871 | Immunoglobulin heavy constant mu (Ig mu chain C region) (Ig mu chain C |
| | region BOT) (Ig mu chain C region GAL) (Ig mu chain C region OU) |
| A6NGN9 | IgLON family member 5 |
| Q8N6C5 | Immunoglobulin superfamily member 1 (IgSF1) (Immunoglobulin-like domain- |
| | containing protein 1) (Inhibin-binding protein) (InhBP) (Pituitary gland-specific |
| | factor 2) (p120) |
| Q5DX21 | Immunoglobulin superfamily member 11 (IgSF11) (Brain and testis-specific |
| | immunoglobulin superfamily protein) (Bt-IGSF) (V-set and immunoglobulin |
| | domain-containing protein 3) |
| Q96ID5 | Immunoglobulin superfamily member 21 (IgSF21) |
| O75054 | Immunoglobulin superfamily member 3 (IgSF3) (Glu-Trp-Ile EWI motif- |
| | containing protein 3) (EWI-3) |
| Q969P0 | Immunoglobulin superfamily member 8 (IgSF8) (CD81 partner 3) (Glu-Trp-Ile |
| | EWI motif-containing protein 2) (EWI-2) (Keratinocytes-associated |
| | transmembrane protein 4) (KCT-4) (LIR-D1) (Prostaglandin regulatory-like |
| | protein) (PGRL) (CD antigen CD316) |
| Q9UPX0 | Protein turtle homolog B (Immunoglobulin superfamily member 9B) (IgSF9B) |
| Q96F46 | Interleukin-17 receptor A (IL-17 receptor A) (IL-17RA) (CDw217) (CD antigen |
| | CD217) |
| Q9NRM6 | Interleukin-17 receptor B (IL-17 receptor B) (IL-17RB) (Cytokine receptor-like |
| | 4) (IL-17 receptor homolog 1) (IL-17Rhl) (IL17Rh1) (Interleukin-17B receptor) |
| | (IL-17B receptor) |
| Q9NZN1 | Interleukin-1 receptor accessory protein-like 1 (IL-1-RAPL-1) (IL-1RAPL-1) |
| | (IL1RAPL-1) (Oligophrenin-4) (Three immunoglobulin domain-containing IL-1 |
| | receptor-related 2) (TIGIRR-2) (X-linked interleukin-1 receptor accessory |
| | protein-like 1) |
| Q01638 | Interleukin-1 receptor-like 1 (Protei?T2) |
| Q8NI17 | Interleukin-31 receptor subunit alpha (IL-31 receptor subunit alpha) (IL-31R |
| | subunit alpha) (IL-31R-alpha) (IL-31RA) (Cytokine receptor-like 3) (GLM-R) |
| | (hGLM-R) (Gp130-like monocyte receptor) (Gp130-like receptor) (ZcytoR17) |
| P40189 | Interleukin-6 receptor subunit beta (IL-6 receptor subunit beta) (IL-6R subunit |
| | beta) (IL-6R-beta) (IL-6RB) (CDw130) (Interleukin-6 signal transducer) |
| | (Membrane glycoprotein 130) (gp130) (Oncostatin-M receptor subunit alpha) |
| | (CD antigen CD130) |
| Q9NX62 | Inositol monophosphatase 3 (IMP 3) (IMPase 3) (EC 3.1.3.25) (EC 3.1.3.7) |
| | (Golgi 3-prime phosphoadenosine 5-prime phosphate 3-prime phosphatase) |
| | (Golgi-resident PAP phosphatase) (gPAPP) (Inositol monophosphatase domain- |
| | containing protein 1) (Inositol-1(or 4)-monophosphatase 3) (Myo-inositol |
| | monophosphatase A3) |
| P06213 | Insulin receptor (IR) (EC 2.7.10.1) (CD antigen CD220) [Cleaved into: Insulin |
| | receptor subunit alpha; Insulin receptor subunit beta] |
| Q6DN90 | IQ motif and SEC7 domain-containing protein 1 (ADP-ribosylation factors |
| | guanine nucleotide-exchange protein 100) (ADP-ribosylation factors guanine |
| | nucleotide-exchange protein 2) (Brefeldin-resistant Arf-GEF 2 protein) |
| | (BRAG2) |
| P48200 | Iron-responsive element-binding protein 2 (IRE-BP 2) (Iron regulatory protein |
| | 2) (IRP2) |
| O14498 | Immunoglobulin superfamily containing leucine-rich repeat protein |
| Q6UXK2 | Immunoglobulin superfamily containing leucine-rich repeat protein 2 (Leucine- |
| | rich repeat domain and immunoglobulin domain-containing axon extension |
| | protein) |
| Q8TB96 | T-cell immunomodulatory protein (Protein TIP) (Integrin-alpha FG-GAP repeat- |
| | containing protein 1) (Linkin) |
| P56199 | Integrin alpha-1 (CD49 antigen-like family member A) (Laminin and collagen |
| | receptor) (VLA-1) (CD antigen CD49a) |
| O75578 | Integrin alpha-10 |
| P17301 | Integrin alpha-2 (CD49 antigen-like family member B) (Collagen receptor) |
| | (Platelet membrane glycoprotein Ia) (GPIa) (VLA-2 subunit alpha) (CD antigen |
| | CD49b) |
| P26006 | Integrin alpha-3 (CD49 antigen-like family member C) (FRP-2) (Galactoprotein |
| | B3) (GAPB3) (VLA-3 subunit alpha) (CD antigen CD49c) [Cleaved into: |
| | Integrin alpha-3 heavy chain; Integrin alpha-3 light chain] |
| P13612 | Integrin alpha-4 (CD49 antigen-like family member D) (Integrin alpha-IV) |
| | (VLA-4 subunit alpha) (CD antigen CD49d) |
| P08648 | Integrin alpha-5 (CD49 antigen-like family member E) (Fibronectin receptor |
| | subunit alpha) (Integrin alpha-F) (VLA-5) (CD antigen CD49e) [Cleaved into: |
| | Integrin alpha-5 heavy chain; Integrin alpha-5 light chain] |
| P23229 | Integrin alpha-6 (CD49 antigen-like family member F) (VLA-6) (CD antigen |
| | CD49f) [Cleaved into: Integrin alpha-6 heavy chain; Integrin alpha-6 light chain; |
| | Processed integrin alpha-6 (Alpha6p)] |
| Q13683 | Integrin alpha-7 [Cleaved into: Integrin alpha-7 heavy chain; Integrin alpha-7 |
| | light chain; Integrin alpha-7 70 kDa form] |
| Q13797 | Integrin alpha-9 (Integrin alpha-RLC) |
| P20701 | Integrin alpha-L (CD11 antigen-like family member A) (Leukocyte adhesion |
| | glycoprotein LFA-1 alpha chain) (LFA-1A) (Leukocyte function-associated |
| | molecule 1 alpha chain) (CD antigen CD11a) |
| P11215 | Integrin alpha-M (CD11 antigen-like family member B) (CR-3 alpha chain) |
| | (Cell surface glycoprotein MAC-1 subunit alpha) (Leukocyte adhesion receptor |
| | MO1) (Neutrophil adherence receptor) (CD antigen CD11b) |
| P06756 | Integrin alpha-V (Vitronectin receptor) (Vitronectin receptor subunit alpha) (CD |
| | antigen CD51) [Cleaved into: Integrin alpha-V heavy chain; Integrin alpha-V |
| | light chain] |
| P05556 | Integrin beta-1 (Fibronectin receptor subunit beta) (Glycoprotein IIa) (GPIIA) |
| | (VLA-4 subunit beta) (CD antigen CD29) |
| P05107 | Integrin beta-2 (Cell surface adhesion glycoproteins LFA-1/CR3/p150, 95 |
| | subunit beta) (Complement receptor C3 subunit beta) (CD antigen CD18) |
| P05106 | Integrin beta-3 (Platelet membrane glycoprotein IIIa) (GPIIIa) (CD antigen |
| | CD61) |
| P16144 | Integrin beta-4 (GP150) (CD antigen CD104) |
| P18084 | Integrin beta-5 |
| P26012 | Integrin beta-8 |
| P19827 | Inter-alpha-trypsin inhibitor heavy chain H1 (ITI heavy chain H1) (ITI-HC1) |
| | (Inter-alpha-inhibitor heavy chain 1) (Inter-alpha-trypsin inhibitor complex |
| | component III) (Serum-derived hyaluronan-associated protein) (SHAP) |
| Q14624 | Inter-alpha-trypsin inhibitor heavy chain H4 (ITI heavy chain H4) (ITI-HC4) |
| | (Inter-alpha-inhibitor heavy chain 4) (Inter-alpha-trypsin inhibitor family heavy |
| | chain-related protein) (IHRP) (Plasma kallikrein sensitive glycoprotein 120) |
| | (Gp120) (PK-120) [Cleaved into: 70 kDa inter-alpha-trypsin inhibitor heavy |
| | chain H4; 35 kDa inter-alpha-trypsin inhibitor heavy chain H4] |
| Q86UX2 | Inter-alpha-trypsin inhibitor heavy chain H5 (ITI heavy chain H5) (ITI-HC5) |
| | (Inter-alpha-inhibitor heavy chain 5) |
| Q9Y287 | Integral membrane protein 2B (Immature BRI2) (imBRI2) (Protein E25B) |
| | (Transmembrane protein BRI) (Bri) [Cleaved into: BRI2, membrane form |
| | (Mature BRI2) (mBRI2); BRI2 intracellular domain (BRI2 ICD); BRI2C, |
| | soluble form; Bri23 peptide (Bri2-23) (ABri23) (C-terminal peptide) (P23 |
| | peptide)] |
| Q14643 | Inositol 1,4,5-trisphosphate receptor type 1 (IP3 receptor isoform 1) (IP3R 1) |
| | (InsP3Rl) (Type 1 inositol 1,4,5-trisphosphate receptor) (Type 1 InsP3 receptor) |
| Q14571 | Inositol 1,4,5-trisphosphate receptor type 2 (IP3 receptor isoform 2) (IP3R 2) |
| | (InsP3R2) (Type 2 inositol 1,4,5-trisphosphate receptor) (Type 2 InsP3 receptor) |
| P57087 | Junctional adhesion molecule B (JAM-B) (Junctional adhesion molecule 2) |
| | (JAM-2) (Vascular endothelial junction-associated molecule) (VE-JAM) (CD |
| | antigen CD322) |
| Q9BX67 | Junctional adhesion molecule C (JAM-C) (JAM-2) (Junctional adhesion |
| | molecule 3) (JAM-3) |
| P01591 | Immunoglobulin J chain (Joining chain of multimeric IgA and IgM) |
| Q9P2N6 | KAT8 regulatory NSL complex subunit 3 (NSL complex protein NSL3) (Non- |
| | specific lethal 3 homolog) (Serum inhibited-related protein) (Testis development |
| | protein PRTD) |
| Q14003 | Potassium voltage-gated channel subfamily C member 3 (KSHIIID) (Voltage- |
| | gated potassium channel subunit Kv3.3) |
| Q03721 | Potassium voltage-gated channel subfamily C member 4 (KSHIIIC) (Voltage- |
| | gated potassium channel subunit Kv3.4) |
| Q8NCM2 | Potassium voltage-gated channel subfamily H member 5 (Ether-a-go-go |
| | potassium channel 2) (hEAG2) (Voltage-gated potassium channel subunit |
| | Kv10.2) |
| Q7Z4H8 | KDEL motif-containing protein 2 |
| P35968 | Vascular endothelial growth factor receptor 2 (VEGFR-2) (EC 2.7.10.1) (Fetal |
| | liver kinase 1) (FLK-1) (Kinase insert domain receptor) (KDR) (Protein-tyrosine |
| | kinase receptor flk-1) (CD antigen CD309) |
| P23276 | Kell blood group glycoprotein (EC 3.4.24.—) (CD antigen CD238) |
| Q8IZA0 | Dyslexia-associated protein KIAA0319-like protein (Adeno-associated virus |
| | receptor) (AAVR) |
| Q2LD37 | Uncharacterized protein KIAA1109 (Fragile site-associated protein) |
| A8MWY0 | UPF0577 protein KIAA1324-like (Estrogen-induced gene 121-like protein) |
| | (hEIG121L) |
| Q6ZVL6 | UPF0606 protein KIAA1549L |
| Q8NI77 | Kinesin-like protein KIF18A (Marrow stromal KIF18A) (MS-KIF18A) |
| Q12756 | Kinesin-like protein KIF1A (Axonal transporter of synaptic vesicles) |
| | (Microtubule-based motor KIF1A) (Unc-104- and KIF1A-related protein) |
| | (hUnc-104) |
| O00139 | Kinesin-like protein KIF2A (Kinesin-2) (hK2) |
| O60282 | Kinesin heavy chain isoform 5C (Kinesin heavy chain neuron-specific 2) |
| P10721 | Mast/stem cell growth factor receptor Kit (SCFR) (EC 2.7.10.1) (Piebald trait |
| | protein) (PBT) (Proto-oncogene c-Kit) (Tyrosine-protein kinase Kit) (p145 c-kit) |
| | (v-kit Hardy-Zuckerman 4 feline sarcoma viral oncogene homolog) (CD antigen |
| | CD117) |
| Q92876 | Kallikrein-6 (EC 3.4.21.—) (Neurosin) (Protease M) (SP59) (Serine protease 18) |
| | (Serine protease 9) (Zyme) |
| P03952 | Plasma kallikrein (EC 3.4.21.34) (Fletcher factor) (Kininogenin) (Plasma |
| | prekallikrein) (PKK) [Cleaved into: Plasma kallikrein heavy chain; Plasma |
| | kallikrein light chain] |
| Q03164 | Histone-lysine N-methyltransferase 2A (Lysine N-methyltransferase 2A) (EC |
| | 2.1.1.43) (ALL-1) (CXXC-type zinc finger protein 7) (Myeloid/lymphoid or |
| | mixed-lineage leukemia) (Myeloid/lymphoid or mixed-lineage leukemia protein |
| | 1) (Trithorax-like protein) (Zinc finger protein HRX) [Cleaved into: MLL |
| | cleavage product N320 (N-terminal cleavage product of 320 kDa) (p320); MLL |
| | cleavage product C180 (C-terminal cleavage product of 180 kDa) (p180)] |
| Q8NEZ4 | Histone-lysine N-methyltransferase 2C (Lysine N-methyltransferase 2C) (EC |
| | 2.1.1.43) (Homologous to ALR protein) (Myeloid/lymphoid or mixed-lineage |
| | leukemia protein 3) |
| P01042 | Kininogen-1 (Alpha-2-thiol proteinase inhibitor) (Fitzgerald factor) (High |
| | molecular weight kininogen) (HMWK) (Williams-Fitzgerald-Flaujeac factor) |
| | [Cleaved into: Kininogen-1 heavy chain; T-kinin (Ile-Ser-Bradykinin); |
| | Bradykinin (Kallidin I); Lysyl-bradykinin (Kallidin II); Kininogen-1 light chain; |
| | Low molecular weight growth-promoting factor] |
| P35908 | Keratin, type II cytoskeletal 2 epidermal (Cytokeratin-2e) (CK-2e) (Epithelial |
| | keratin-2e) (Keratin-2 epidermis) (Keratin-2e) (K2e) (Type-II keratin Kb2) |
| P35527 | Keratin, type I cytoskeletal 9 (Cytokeratin-9) (CK-9) (Keratin-9) (K9) |
| P32004 | Neural cell adhesion molecule L1 (N-CAM-L1) (NCAM-L1) (CD antigen |
| | CD171) |
| P25391 | Laminin subunit alpha-1 (Laminin A chain) (Laminin-1 subunit alpha) |
| | (Laminin-3 subunit alpha) (S-laminin subunit alpha) (S-LAM alpha) |
| P24043 | Laminin subunit alpha-2 (Laminin M chain) (Laminin-12 subunit alpha) |
| | (Laminin-2 subunit alpha) (Laminin-4 subunit alpha) (Merosin heavy chain) |
| Q16363 | Laminin subunit alpha-4 (Laminin-14 subunit alpha) (Laminin-8 subunit alpha) |
| | (Laminin-9 subunit alpha) |
| O15230 | Laminin subunit alpha-5 (Laminin-10 subunit alpha) (Laminin-11 subunit alpha) |
| | (Laminin-15 subunit alpha) |
| P55268 | Laminin subunit beta-2 (Laminin B1s chain) (Laminin-11 subunit beta) |
| | (Laminin-14 subunit beta) (Laminin-15 subunit beta) (Laminin-3 subunit beta) |
| | (Laminin-4 subunit beta) (Laminin-7 subunit beta) (Laminin-9 subunit beta) (S- |
| | laminin subunit beta) (S-LAM beta) |
| P11047 | Laminin subunit gamma-1 (Laminin B2 chain) (Laminin-1 subunit gamma) |
| | (Laminin-10 subunit gamma) (Laminin-11 subunit gamma) (Laminin-2 subunit |
| | gamma) (Laminin-3 subunit gamma) (Laminin-4 subunit gamma) (Laminin-6 |
| | subunit gamma) (Laminin-7 subunit gamma) (Laminin-8 subunit gamma) |
| | (Laminin-9 subunit gamma) (S-laminin subunit gamma) (S-LAM gamma) |
| Q9Y6N6 | Laminin subunit gamma-3 (Laminin-12 subunit gamma) (Laminin-14 subunit |
| | gamma) (Laminin-15 subunit gamma) |
| P11279 | Lysosome-associated membrane glycoprotein 1 (LAMP-1) (Lysosome- |
| | associated membrane protein 1) (CD107 antigen-like family member A) (CD |
| | antigen CD107a) |
| P13473 | Lysosome-associated membrane glycoprotein 2 (LAMP-2) (Lysosome- |
| | associated membrane protein 2) (CD107 antigen-like family member B) (LGP- |
| | 96) (CD antigen CD107b) |
| Q9UJQ1 | Lysosome-associated membrane glycoprotein 5 (Brain and dendritic cell- |
| | associated LAMP) (Brain-associated LAMP-like protein) (BAD-LAMP) |
| | (Lysosome-associated membrane protein 5) (LAMP-5) |
| O95461 | LARGE xylosyl- and glucuronyltransferase 1 (EC 2.4.—.—) |
| | (Acetylglucosaminyltransferase-like 1A) (Glycosyltransferase-like protein) |
| | [Includes: Xylosyltransferase LARGE (EC 2.4.2.—); Beta-1,3- |
| | glucuronyltransferase LARGE (EC 2.4.1.—)] |
| P07195 | L-lactate dehydrogenase B chain (LDH-B) (EC 1.1.1.27) (LDH heart subunit) |
| | (LDH-H) (Renal carcinoma antigen NY-REN-46) |
| P09382 | Galectin-1 (Gal-1) (14 kDa laminin-binding protein) (HLBP14) (14 kDa lectin) |
| | (Beta-galactoside-binding lectin L-14-I) (Galaptin) (HBL) (HPL) (Lactose- |
| | binding lectin 1) (Lectin galactoside-binding soluble 1) (Putative MAPK- |
| | activating protein PM12) (S-Lac lectin 1) |
| Q08380 | Galectin-3-binding protein (Basement membrane autoantigen p105) (Lectin |
| | galactoside-binding soluble 3-binding protein) (Mac-2-binding protein) |
| | (MAC2BP) (Mac-2 BP) (Tumor-associated antigen 90K) |
| O95970 | Leucine-rich glioma-inactivated protein 1 (Epitempin-1) |
| Q8N0V4 | Leucine-rich repeat LGI family member 2 (LGI1-like protein 2) (Leucine-rich |
| | glioma-inactivated protein 2) |
| Q99538 | Legumain (EC 3.4.22.34) (Asparaginyl endopeptidase) (Protease, cysteine 1) |
| P42702 | Leukemia inhibitory factor receptor (LIF receptor) (LIF-R) (CD antigen CD118) |
| Q96FE5 | Leucine-rich repeat and immunoglobulin-like domain-containing nogo receptor- |
| | interacting protein 1 (Leucine-rich repeat and immunoglobulin domain- |
| | containing protein 1) (Leucine-rich repeat neuronal protein 1) (Leucine-rich |
| | repeat neuronal protein 6A) |
| Q7L985 | Leucine-rich repeat and immunoglobulin-like domain-containing nogo receptor- |
| | interacting protein 2 (Leucine-rich repeat neuronal protein 3) (Leucine-rich |
| | repeat neuronal protein 6C) |
| Q8NG48 | Protein Lines homolog 1 (Wnt-signaling molecule Lines homolog 1) |
| Q9NUN5 | Probable lysosomal cobalamin transporter (HDAg-L-interacting protein NESI) |
| | (LMBR1 domain-containing protein 1) (Nuclear export signal-interacting |
| | protein) |
| Q9BU23 | Lipase maturation factor 2 (Transmembrane protein 112B) (Transmembrane |
| | protein 153) |
| Q03252 | Lamin-B2 |
| P25800 | Rhombotin-1 (Cysteine-rich protein TTG-1) (LIM domain only protein 1) |
| | (LMO-1) (T-cell translocation protein 1) |
| Q9UIQ6 | Leucyl-cystinyl aminopeptidase (Cystinyl aminopeptidase) (EC 3.4.11.3) |
| | (Insulin-regulated membrane aminopeptidase) (Insulin-responsive |
| | aminopeptidase) (IRAP) (Oxytocinase) (OTase) (Placental leucine |
| | aminopeptidase) (P-LAP) [Cleaved into: Leucyl-cystinyl aminopeptidase, |
| | pregnancy serum form] |
| P36776 | Lon protease homolog, mitochondrial (EC 3.4.21.53) (LONHs) (Lon protease- |
| | like protein) (LONP) (Mitochondrial ATP-dependent protease Lon) (Serine |
| | protease 15) |
| Q93052 | Lipoma-preferred partner (LIM domain-containing preferred translocation |
| | partner in lipoma) |
| P02750 | Leucine-rich alpha-2-glycoprotein (LRG) |
| Q96JA1 | Leucine-rich repeats and immunoglobulin-like domains protein 1 (LIG-1) |
| Q07954 | Prolow-density lipoprotein receptor-related protein 1 (LRP-1) (Alpha-2- |
| | macroglobulin receptor) (A2MR) (Apolipoprotein E receptor) (APOER) (CD |
| | antigen CD91) [Cleaved into: Low-density lipoprotein receptor-related protein 1 |
| | 85 kDa subunit (LRP-85); Low-density lipoprotein receptor-related protein 1 |
| | 515 kDa subunit (LRP-515); Low-density lipoprotein receptor-related protein 1 |
| | intracellular domain (LRPICD)] |
| Q9NZR2 | Low-density lipoprotein receptor-related protein 1B (LRP-1B) (Low-density |
| | lipoprotein receptor-related protein-deleted in tumor) (LRP-DIT) |
| P98164 | Low-density lipoprotein receptor-related protein 2 (LRP-2) (Glycoprotein 330) |
| | (gp330) (Megalin) |
| O75096 | Low-density lipoprotein receptor-related protein 4 (LRP-4) (Multiple epidermal |
| | growth factor-like domains 7) |
| P30533 | Alpha-2-macroglobulin receptor-associated protein (Alpha-2-MRAP) (Low |
| | density lipoprotein receptor-related protein-associated protein 1) (RAP) |
| Q9HBW1 | Leucine-rich repeat-containing protein 4 (Brain tumor-associated protein BAG) |
| | (Nasopharyngeal carcinoma-associated gene 14 protein) (Netrin-G2 ligand) |
| | (NGL-2) |
| Q9NT99 | Leucine-rich repeat-containing protein 4B (Netrin-G3 ligand) (NGL-3) |
| Q9HCJ2 | Leucine-rich repeat-containing protein 4C (Netrin-G1 ligand) (NGL-1) |
| Q7Z2Q7 | Leucine-rich repeat-containing protein 70 (Synleurin) |
| Q8IWT6 | Volume-regulated anion channel subunit LRRC8A (Leucine-rich repeat- |
| | containing protein 8A) (Swelling protein 1) |
| Q6P9F7 | Volume-regulated anion channel subunit LRRC8B (Leucine-rich repeat- |
| | containing protein 8B) (T-cell activation leucine repeat-rich protein) (TA-LRRP) |
| Q6ZRR7 | Leucine-rich repeat-containing protein 9 |
| Q6UXK5 | Leucine-rich repeat neuronal protein 1 (Neuronal leucine-rich repeat protein 1) |
| | (NLRR-1) |
| Q8WUT4 | Leucine-rich repeat neuronal protein 4 (Neuronal leucine-rich repeat protein 4) |
| | (NLRR-4) |
| O43300 | Leucine-rich repeat transmembrane neuronal protein 2 (Leucine-rich repeat |
| | neuronal 2 protein) |
| Q86VH4 | Leucine-rich repeat transmembrane neuronal protein 4 |
| Q8N967 | Leucine-rich repeat and transmembrane domain-containing protein 2 |
| Q13449 | Limbic system-associated membrane protein (LSAMP) (IgLON family member |
| | 3) |
| Q14766 | Latent-transforming growth factor beta-binding protein 1 (LTBP-1) |
| | (Transforming growth factor beta-1-binding protein 1) (TGF-beta1-BP-1) |
| Q14767 | Latent-transforming growth factor beta-binding protein 2 (LTBP-2) |
| P02788 | Lactotransferrin (Lactoferrin) (EC 3.4.21.—) (Growth-inhibiting protein 12) |
| | (Talalactoferrin) [Cleaved into: Lactoferricin-H (Lfcin-H); Kaliocin-1; |
| | Lactoferroxin-A; Lactoferroxin-B; Lactoferroxin-C] |
| P51884 | Lumican (Keratan sulfate proteoglycan lumican) (KSPG lumican) |
| O60449 | Lymphocyte antigen 75 (Ly-75) (C-type lectin domain family 13 member B) |
| | (DEC-205) (gp200-MR6) (CD antigen CD205) |
| Q9Y5Y7 | Lymphatic vessel endothelial hyaluronic acid receptor 1 (LYVE-1) (Cell surface |
| | retention sequence-binding protein 1) (CRSBP-1) (Extracellular link domain- |
| | containing protein 1) (Hyaluronic acid receptor) |
| P20645 | Cation-dependent mannose-6-phosphate receptor (CD Man-6-P receptor) (CD- |
| | MPR) (46 kDa mannose 6-phosphate receptor) (MPR 46) |
| Q9UPN3 | Microtubule-actin cross-linking factor 1, isoforms 1/2/3/5 (620 kDa actin- |
| | binding protein) (ABP620) (Actin cross-linking family protein 7) (Macrophin-1) |
| | (Trabeculin-alpha) |
| Q8WXG6 | MAP kinase-activating death domain protein (Differentially expressed in normal |
| | and neoplastic cells) (Insulinoma glucagonoma clone 20) (Rab3 GDP/GTP |
| | exchange factor) |
| P20916 | Myelin-associated glycoprotein (Siglec-4a) |
| P20794 | Serine/threonine-protein kinase MAK (EC 2.7.11.1) (Male germ cell-associated |
| | kinase) |
| Q8IZL2 | Mastermind-like protein 2 (Mam-2) |
| P49641 | Alpha-mannosidase 2x (EC 3.2.1.114) (Alpha-mannosidase IIx) (Man IIx) |
| | (Mannosidase alpha class 2A member 2) (Mannosyl-oligosaccharide 1,3-1,6- |
| | alpha-mannosidase) |
| O00754 | Lysosomal alpha-mannosidase (Laman) (EC 3.2.1.24) (Lysosomal acid alpha- |
| | mannosidase) (Mannosidase alpha class 2B member 1) (Mannosidase alpha-B) |
| | [Cleaved into: Lysosomal alpha-mannosidase A peptide; Lysosomal alpha- |
| | mannosidase B peptide; Lysosomal alpha-mannosidase C peptide; Lysosomal |
| | alpha-mannosidase D peptide; Lysosomal alpha-mannosidase E peptide] |
| Q9Y2E5 | Epididymis-specific alpha-mannosidase (EC 3.2.1.24) (Mannosidase alpha class |
| | 2B member 2) |
| P11137 | Microtubule-associated protein 2 (MAP-2) |
| Q02750 | Dual specificity mitogen-activated protein kinase kinase 1 (MAP kinase kinase |
| | 1) (MAPKK 1) (MKK1) (EC 2.7.12.2) (ERK activator kinase 1) (MAPK/ERK |
| | kinase 1) (MEK 1) |
| P41279 | Mitogen-activated protein kinase kinase kinase 8 (EC 2.7.11.25) (Cancer Osaka |
| | thyroid oncogene) (Proto-oncogene c-Cot) (Serine/threonine-protein kinase cot) |
| | (Tumor progression locus 2) (TPL-2) |
| P10636 | Microtubule-associated protein tau (Neurofibrillary tangle protein) (Paired |
| | helical filament-tau) (PHF-tau) |
| P29966 | Myristoylated alanine-rich C-kinase substrate (MARCKS) (Protein kinase C |
| | substrate, 80 kDa protein, light chain) (80K-L protein) (PKCSL) |
| P49006 | MARCKS-related protein (MARCKS-like protein 1) (Macrophage |
| | myristoylated alanine-rich C kinase substrate) (Mac-MARCKS) |
| | (MacMARCKS) |
| Q9UEW3 | Macrophage receptor MARCO (Macrophage receptor with collagenous |
| | structure) (Scavenger receptor class A member 2) |
| P02686 | Myelin basic protein (MBP) (Myelin A1 protein) (Myelin membrane |
| | encephalitogenic protein) |
| P43121 | Cell surface glycoprotein MUC18 (Cell surface glycoprotein P1H12) |
| | (Melanoma cell adhesion molecule) (Melanoma-associated antigen A32) |
| | (Melanoma-associated antigen MUC18) (S-endo 1 endothelial-associated |
| | antigen) (CD antigen CD146) |
| Q9BTE3 | Mini-chromosome maintenance complex-binding protein (MCM-BP) (MCM- |
| | binding protein) |
| Q8NFP4 | MAM domain-containing glycosylphosphatidylinositol anchor protein 1 (GPI |
| | and MAM protein) (GPIM) (Glycosylphosphatidylinositol-MAM) (MAM |
| | domain-containing protein 3) |
| Q7Z553 | MAM domain-containing glycosylphosphatidylinositol anchor protein 2 (MAM |
| | domain-containing protein 1) |
| P40925 | Malate dehydrogenase, cytoplasmic (EC 1.1.1.37) (Cytosolic malate |
| | dehydrogenase) (Diiodophenylpyruvate reductase) (EC 1.1.1.96) |
| Q96KG7 | Multiple epidermal growth factor-like domains protein 10 (Multiple EGF-like |
| | domains protein 10) |
| Q7Z7M0 | Multiple epidermal growth factor-like domains protein 8 (Multiple EGF-like |
| | domains protein 8) (Epidermal growth factor-like protein 4) (EGF-like protein 4) |
| Q9H1U4 | Multiple epidermal growth factor-like domains protein 9 (Multiple EGF-like |
| | domains protein 9) (Epidermal growth factor-like protein 5) (EGF-like protein 5) |
| P08582 | Melanotransferrin (Melanoma-associated antigen p97) (CD antigen CD228) |
| Q12866 | Tyrosine-protein kinase Mer (EC 2.7.10.1) (Proto-oncogene c-Mer) (Receptor |
| | tyrosine kinase MerTK) |
| P08581 | Hepatocyte growth factor receptor (HGF receptor) (EC 2.7.10.1) (HGF/SF |
| | receptor) (Proto-oncogene c-Met) (Scatter factor receptor) (SF receptor) |
| | (Tyrosine-protein kinase Met) |
| Q9BUU2 | Methyltransferase-like protein 22 (EC 2.1.1.—) |
| O75121 | Microfibrillar-associated protein 3-like (Testis development protein NYD-SP9) |
| P55083 | Microfibril-associated glycoprotein 4 |
| Q08431 | Lactadherin (Breast epithelial antigen BA46) (HMFG) (MFGM) (Milk fat |
| | globule-EGF factor 8) (MFG-E8) (SED1) [Cleaved into: Lactadherin short form; |
| | Medin] |
| O60291 | E3 ubiquitin-protein ligase MGRN1 (EC 2.3.2.27) (Mahogunin RING finger |
| | protein 1) (RING finger protein 156) (RING-type E3 ubiquitin transferase |
| | MGRN1) |
| Q5JRA6 | Transport and Golgi organization protein 1 homolog (TANGO1) (C219-reactive |
| | peptide) (D320) (Melanoma inhibitory activity protein 3) |
| Q9UNW1 | Multiple inositol polyphosphate phosphatase 1 (EC 3.1.3.62) (2,3- |
| | bisphosphoglycerate 3-phosphatase) (2,3-BPG phosphatase) (EC 3.1.3.80) |
| | (Inositol (1,3,4,5)-tetrakisphosphate 3-phosphatase) (Ins(1,3,4,5)P(4) 3- |
| | phosphatase) |
| Q13201 | Multimerin-1 (EMILIN-4) (Elastin microfibril interface located protein 4) |
| | (Elastin microfibril interfacer 4) (Endothelial cell multimerin) [Cleaved into: |
| | Platelet glycoprotein Ia*; 155 kDa platelet multimerin (p-155) (p155)] |
| Q9H8L6 | Multimerin-2 (EMILIN-3) (Elastin microfibril interface located protein 3) |
| | (Elastin microfibril interfacer 3) (EndoGlyx-1 p125/p140 subunit) |
| P41218 | Myeloid cell nuclear differentiation antigen |
| Q16653 | Myelin-oligodendrocyte glycoprotein |
| Q6UVY6 | DBH-like monooxygenase protein 1 (EC 1.14.17.—) (Monooxygenase X) |
| P05164 | Myeloperoxidase (MPO) (EC 1.11.2.2) [Cleaved into: Myeloperoxidase; 89 kDa |
| | myeloperoxidase; 84 kDa myeloperoxidase; Myeloperoxidase light chain; |
| | Myeloperoxidase heavy chain] |
| P25189 | Myelin protein P0 (Myelin peripheral protein) (MPP) (Myelin protein zero) |
| O95297 | Myelin protein zero-like protein 1 (Protein zero-related) |
| O60487 | Myelin protein zero-like protein 2 (Epithelial V-like antigen 1) |
| P22897 | Macrophage mannose receptor 1 (MMR) (C-type lectin domain family 13 |
| | member D) (C-type lectin domain family 13 member D-like) (Human mannose |
| | receptor) (hMR) (Macrophage mannose receptor 1-like protein 1) (CD antigen |
| | CD206) |
| Q9UBG0 | C-type mannose receptor 2 (C-type lectin domain family 13 member E) |
| | (Endocytic receptor 180) (Macrophage mannose receptor 2) (Urokinase-type |
| | plasminogen activator receptor-associated protein) (UPAR-associated protein) |
| | (Urokinase receptor-associated protein) (CD antigen CD280) |
| P21757 | Macrophage scavenger receptor types I and II (Macrophage acetylated LDL |
| | receptor I and II) (Scavenger receptor class A member 1) (CD antigen CD204) |
| P98088 | Mucin-5AC (MUC-5AC) (Gastric mucin) (Lewis B blood group antigen) (LeB) |
| | (Major airway glycoprotein) (Mucin-5 subtype AC, tracheobronchial) |
| | (Tracheobronchial mucin) (TBM) |
| Q9UIF7 | Adenine DNA glycosylase (EC 3.2.2.—) (MutY homolog) (hMYH) |
| P19105 | Myosin regulatory light chain 12A (Epididymis secretory protein Li 24) (HEL- |
| | S-24) (MLC-2B) (Myosin RLC) (Myosin regulatory light chain 2, |
| | nonsarcomeric) (Myosin regulatory light chain MRLC3) |
| Q92614 | Unconventional myosin-XVIIIa (Molecule associated with JAK3 N-terminus) |
| | (MAIN) (Myosin containing a PDZ domain) (Surfactant protein receptor SP- |
| | R210) (SP-R210) |
| Q6NSJ0 | Myogenesis-regulating glycosidase (EC 3.2.1.—) (Uncharacterized family 31 |
| | glucosidase KIAA1161) |
| Q02083 | N-acylethanolamine-hydrolyzing acid amidase (EC 3.5.1.-) (Acid ceramidase- |
| | like protein) (N-acylsphingosine amidohydrolase-like) (ASAH-like protein) |
| | [Cleaved into: N-acylethanolamine-hydrolyzing acid amidase subunit alpha; N- |
| | acylethanolamine-hydrolyzing acid amidase subunit beta] |
| Q9Y3Q0 | N-acetylated-alpha-linked acidic dipeptidase 2 (EC 3.4.17.21) (Glutamate |
| | carboxypeptidase III) (GCPIII) (N-acetylated-alpha-linked acidic dipeptidase II) |
| | (NAALADase II) |
| P17050 | Alpha-N-acetylgalactosaminidase (EC 3.2.1.49) (Alpha-galactosidase B) |
| P54920 | Alpha-soluble NSF attachment protein (SNAP-alpha) (N-ethylmaleimide- |
| | sensitive factor attachment protein alpha) |
| Q9H115 | Beta-soluble NSF attachment protein (SNAP-beta) (N-ethylmaleimide-sensitive |
| | factor attachment protein beta) |
| Q8IW45 | ATP-dependent (S)-NAD(P)H-hydrate dehydratase (EC 4.2.1.93) (ATP- |
| | dependent NAD(P)HX dehydratase) (Carbohydrate kinase domain-containing |
| | protein) (NAD(P)HX dehydratase) |
| P41271 | Neuroblastoma suppressor of tumorigenicity 1 (DAN domain family member 1) |
| | (Protein N03) (Zinc finger protein DAN) |
| P13591 | Neural cell adhesion molecule 1 (N-CAM-1) (NCAM-1) (CD antigen CD56) |
| O15394 | Neural cell adhesion molecule 2 (N-CAM-2) (NCAM-2) |
| O14594 | Neurocan core protein (Chondroitin sulfate proteoglycan 3) |
| Q9UBB6 | Neurochondrin |
| Q6PIU2 | Neutral cholesterol ester hydrolase 1 (NCEH) (EC 3.1.1.—) (Arylacetamide |
| | deacetylase-like 1) |
| Q969V3 | Nicalin (Nicastrin-like protein) |
| Q15788 | Nuclear receptor coactivator 1 (NCoA-1) (EC 2.3.1.48) (Class E basic helix- |
| | loop-helix protein 74) (bHLHe74) (Protein Hin-2) (RIP160) (Renal carcinoma |
| | antigen NY-REN-52) (Steroid receptor coactivator 1) (SRC-1) |
| Q15596 | Nuclear receptor coactivator 2 (NCoA-2) (Class E basic helix-loop-helix protein |
| | 75) (bHLHe75) (Transcriptional intermediary factor 2) (hTIF2) |
| Q14686 | Nuclear receptor coactivator 6 (Activating signal cointegrator 2) (ASC-2) |
| | (Amplified in breast cancer protein 3) (Cancer-amplified transcriptional |
| | coactivator ASC-2) (Nuclear receptor coactivator RAP250) (NRC RAP250) |
| | (Nuclear receptor-activating protein, 250 kDa) (Peroxisome proliferator- |
| | activated receptor-interacting protein) (PPAR-interacting protein) (PRIP) |
| | (Thyroid hormone receptor-binding protein) |
| Q92542 | Nicastrin |
| Q8TB73 | Protein NDNF (Neuron-derived neurotrophic factor) |
| Q9ULP0 | Protein NDRG4 (Brain development-related molecule 1) (N-myc downstream- |
| | regulated gene 4 protein) (Vascular smooth muscle cell-associated protein 8) |
| | (SMAP-8) |
| O95299 | NADH dehydrogenase [ubiquinone] 1 alpha subcomplex subunit 10, |
| | mitochondrial (Complex I-42 kD) (CI-42 kD) (NADH-ubiquinone oxidoreductase |
| | 42 kDa subunit) |
| Q15223 | Nectin-1 (Herpes virus entry mediator C) (Herpesvirus entry mediator C) |
| | (HveC) (Herpesvirus Ig-like receptor) (HIgR) (Nectin cell adhesion molecule 1) |
| | (Poliovirus receptor-related protein 1) (CD antigen CD111) |
| Q9NQS3 | Nectin-3 (CDw113) (Nectin cell adhesion molecule 3) (Poliovirus receptor- |
| | related protein 3) (CD antigen CD113) |
| Q7Z3B1 | Neuronal growth regulator 1 (IgLON family member 4) |
| Q99435 | Protein kinase C-binding protein NELL2 (NEL-like protein 2) (Nel-related |
| | protein 2) |
| Q92859 | Neogenin (Immunoglobulin superfamily DCC subclass member 2) |
| Q8TDF5 | Neuropilin and tolloid-like protein 1 (Brain-specific transmembrane protein |
| | containing 2 CUB and 1 LDL-receptor class A domains protein 1) |
| O94856 | Neurofascin |
| Q6P4R8 | Nuclear factor related to kappa-B-binding protein (DNA-binding protein R |
| | kappa-B) (INO80 complex subunit G) |
| Q5JS37 | NHL repeat-containing protein 3 |
| Q14112 | Nidogen-2 (NID-2) (Osteonidogen) |
| Q86X76 | Deaminated glutathione amidase (dGSH amidase) (EC 3.5.1.—) (Nitrilase |
| | homolog 1) |
| Q8N8D7 | Sodium/potassium-transporting ATPase subunit beta-1-interacting protein 3 |
| | (Na(+)/K(+)-transporting ATPase subunit beta-1-interacting protein 3) (Protein |
| | FAM77D) |
| Q8N2Q7 | Neuroligin-1 |
| Q8N0W4 | Neuroligin-4, X-linked (Neuroligin X) (HNLX) |
| P69849 | Nodal modulator 3 (pM5 protein 3) |
| O15118 | Niemann-Pick C1 protein |
| Q8TAT6 | Nuclear protein localization protein 4 homolog (Protein NPL4) |
| Q9Y639 | Neuroplastin (Stromal cell-derived receptor 1) (SDR-1) |
| Q15818 | Neuronal pentraxin-1 (NP1) (Neuronal pentraxin I) (NP-I) |
| P47972 | Neuronal pentraxin-2 (NP2) (Neuronal pentraxin II) (NP-II) |
| O95502 | Neuronal pentraxin receptor |
| Q92823 | Neuronal cell adhesion molecule (Nr-CAM) (Neuronal surface protein Bravo) |
| | (hBravo) (NgCAM-related cell adhesion molecule) (Ng-CAM-related) |
| O14786 | Neuropilin-1 (Vascular endothelial cell growth factor 165 receptor) (CD antigen |
| | CD304) |
| O60462 | Neuropilin-2 (Vascular endothelial cell growth factor 165 receptor 2) |
| Q9ULB1 | Neurexin-1 (Neurexin I-alpha) (Neurexin-1-alpha) |
| P58401 | Neurexin-2-beta (Neurexin II-beta) |
| Q9Y4C0 | Neurexin-3 (Neurexin Ill-alpha) (Neurexin-3-alpha) |
| P21589 | 5′-nucleotidase (5′-NT) (EC 3.1.3.5) (Ecto-5′-nucleotidase) (CD antigen CD73) |
| Q9P121 | Neurotrimin (hNT) (IgLON family member 2) |
| Q96CW9 | Netrin-G2 (Laminet-2) |
| Q16620 | BDNF/NT-3 growth factors receptor (EC 2.7.10.1) (GP145-TrkB) (Trk-B) |
| | (Neurotrophic tyrosine kinase receptor type 2) (TrkB tyrosine kinase) |
| | (Tropomyosin-related kinase B) |
| Q16288 | NT-3 growth factor receptor (EC 2.7.10.1) (GP145-TrkC) (Trk-C) |
| | (Neurotrophic tyrosine kinase receptor type 3) (TrkC tyrosine kinase) |
| Q8TEM1 | Nuclear pore membrane glycoprotein 210 (Nuclear pore protein gp210) (Nuclear |
| | envelope pore membrane protein POM 210) (POM210) (Nucleoporin Nup210) |
| | (Pore membrane protein of 210 kDa) |
| P58417 | Neurexophilin-1 |
| O95156 | Neurexophilin-2 |
| O95158 | Neurexophilin-4 |
| Q02218 | 2-oxoglutarate dehydrogenase, mitochondrial (EC 1.2.4.2) (2-oxoglutarate |
| | dehydrogenase complex component E1) (OGDC-E1) (Alpha-ketoglutarate |
| | dehydrogenase) |
| Q99784 | Noelin (Neuronal olfactomedin-related ER localized protein) (Olfactomedin-1) |
| O95897 | Noelin-2 (Olfactomedin-2) |
| Q96PB7 | Noelin-3 (Olfactomedin-3) (Optimedin) |
| Q6UWY5 | Olfactomedin-like protein 1 |
| Q9NRN5 | Olfactomedin-like protein 3 (HNOEL-iso) (hOLF44) |
| P23515 | Oligodendrocyte-myelin glycoprotein |
| Q14982 | Opioid-binding protein/cell adhesion molecule (OBCAM) (OPCML) (Opioid- |
| | binding cell adhesion molecule) (IgLON family member 1) |
| Q8NH83 | Olfactory receptor 4A5 (Olfactory receptor OR11-111) |
| P02763 | Alpha-1-acid glycoprotein 1 (AGP 1) (Orosomucoid-1) (OMD 1) |
| P19652 | Alpha-1-acid glycoprotein 2 (AGP 2) (Orosomucoid-2) (OMD 2) |
| Q86WC4 | Osteopetrosis-associated transmembrane protein 1 (Chloride channel 7 beta |
| | subunit) |
| Q6ZRI0 | Otogelin |
| Q99571 | P2X purinoceptor 4 (P2X4) (ATP receptor) (Purinergic receptor) |
| Q99572 | P2X purinoceptor 7 (P2X7) (ATP receptor) (P2Z receptor) (Purinergic receptor) |
| Q92791 | Endoplasmic reticulum protei?C65 (Leprecan-like protein 4) (Nucleolar |
| | autoantigen No55) (Prolyl 3-hydroxylase family member 4) (Synaptonemal |
| | complex protei?C65) |
| P13674 | Prolyl 4-hydroxylase subunit alpha-1 (4-PH alpha-1) (EC 1.14.11.2) |
| | (Procollagen-proline, 2-oxoglutarate-4-dioxygenase subunit alpha-1) |
| Q9NXG6 | Transmembrane prolyl 4-hydroxylase (P4H-TM) (EC 1.14.11.—) (Hypoxia- |
| | inducible factor prolyl hydroxylase 4) (HIF-PH4) (HIF-prolyl hydroxylase 4) |
| | (HPH-4) |
| Q9BY11 | Protein kinase C and casein kinase substrate in neurons protein 1 (Syndapin-1) |
| P43034 | Platelet-activating factor acetylhydrolase IB subunit alpha (Lissencephaly-1 |
| | protein) (LIS-1) (PAF acetylhydrolase 45 kDa subunit) (PAF-AH 45 kDa |
| | subunit) (PAF-AH alpha) (PAFAH alpha) |
| Q96RD7 | Pannexin-1 |
| Q9UKK3 | Poly [ADP-ribose] polymerase 4 (PARP-4) (EC 2.4.2.30) (193 kDa vault |
| | protein) (ADP-ribosyltransferase diphtheria toxin-like 4) (ARTD4) (PARP- |
| | related/IalphaI-related H5/proline-rich) (PH5P) (Vault poly(ADP-ribose) |
| | polymerase) (VPARP) |
| Q86U86 | Protein polybromo-1 (hPB1) (BRG1-associated factor 180) (BAF180) |
| | (Polybromo-1D) |
| Q96AQ6 | Pre-B-cell leukemia transcription factor-interacting protein 1 (Hematopoietic |
| | PBX-interacting protein) |
| Q15365 | Poly(rC)-binding protein 1 (Alpha-CP1) (Heterogeneous nuclear |
| | ribonucleoprotein E1) (hnRNP E1) (Nucleic acid-binding protein SUB2.3) |
| Q15366 | Poly(rC)-binding protein 2 (Alpha-CP2) (Heterogeneous nuclear |
| | ribonucleoprotein E2) (hnRNP E2) |
| Q08174 | Protocadherin-1 (Cadherin-like protein 1) (Protocadherin-42) (PC42) |
| O14917 | Protocadherin-17 (Protocadherin-68) |
| Q8TAB3 | Protocadherin-19 |
| O60245 | Protocadherin-7 (Brain-heart protocadherin) (BH-Pcdh) |
| O95206 | Protocadherin-8 (Arcadlin) |
| Q9HC56 | Protocadherin-9 |
| Q9UN70 | Protocadherin gamma-C3 (PCDH-gamma-C3) (Protocadherin-2) |
| | (Protocadherin-43) (PC-43) |
| Q9Y5F6 | Protocadherin gamma-C5 (PCDH-gamma-C5) |
| Q15154 | Pericentriolar material 1 protein (PCM-1) (hPCM-1) |
| P16519 | Neuroendocrine convertase 2 (NEC 2) (EC 3.4.21.94) (KEX2-like endoprotease |
| | 2) (Prohormone convertase 2) (Proprotein convertase 2) (PC2) |
| Q9UHG3 | Prenylcysteine oxidase 1 (EC 1.8.3.5) (Prenylcysteine lyase) |
| P16284 | Platelet endothelial cell adhesion molecule (PECAM-1) (EndoCAM) (GPIIA′) |
| | (PECA1) (CD antigen CD31) |
| P17858 | ATP-dependent 6-phosphofructokinase, liver type (ATP-PFK) (PFK-L) (EC |
| | 2.7.1.11) (6-phosphofructokinase type B) (Phosphofructo-1-kinase isozyme B) |
| | (PFK-B) (Phosphohexokinase) |
| Q75T13 | GPI inositol-deacylase (EC 3.1.—.—) (Post-GPI attachment to proteins factor 1) |
| | (hPGAP1) |
| Q6UXB8 | Peptidase inhibitor 16 (PI-16) (Cysteine-rich secretory protein 9) (CRISP-9) |
| | (PSP94-binding protein) |
| P01833 | Polymeric immunoglobulin receptor (PIgR) (Poly-Ig receptor) (Hepatocellular |
| | carcinoma-associated protein TB6) [Cleaved into: Secretory component] |
| Q96S52 | GPI transamidase component PIG-S (Phosphatidylinositol-glycan biosynthesis |
| | class S protein) |
| Q969N2 | GPI transamidase component PIG-T (Phosphatidylinositol-glycan biosynthesis |
| | class T protein) |
| Q9UKJ1 | Paired immunoglobulin-like type 2 receptor alpha (Cell surface receptor FDF03) |
| | (Inhibitory receptor PILR-alpha) |
| Q99755 | Phosphatidylinositol 4-phosphate 5-kinase type-1 alpha (PIP5K1-alpha) |
| | (PtdIns(4)P-5-kinase 1 alpha) (EC 2.7.1.68) (68 kDa type I phosphatidylinositol |
| | 4-phosphate 5-kinase alpha) (Phosphatidylinositol 4-phosphate 5-kinase type I |
| | alpha) (PIP5KIalpha) |
| Q7Z443 | Polycystic kidney disease protein 1-like 3 (PCI-like 3 protein) (Polycystin-1L3) |
| P14618 | Pyruvate kinase PKM (EC 2.7.1.40) (Cytosolic thyroid hormone-binding |
| | protein) (CTHBP) (Opa-interacting protein 3) (OIP-3) (Pyruvate kinase 2/3) |
| | (Pyruvate kinase muscle isozyme) (Thyroid hormone-binding protein 1) |
| | (THBP1) (Tumor M2-PK) (p58) |
| Q8NCC3 | Group XV phospholipase A2 (EC 2.3.1.—) (1-O-acylceramide synthase) (ACS) |
| | (LCAT-like lysophospholipase) (LLPL) (Lysophospholipase 3) (Lysosomal |
| | phospholipase A2) (LPLA2) |
| Q03405 | Urokinase plasminogen activator surface receptor (U-PAR) (uPAR) (Monocyte |
| | activation antigen Mo3) (CD antigen CD87) |
| Q6P4A8 | Phospholipase B-like 1 (EC 3.1.1.—) (LAMA-like protein 1) (Lamina ancestor |
| | homolog 1) (Phospholipase B domain-containing protein 1) [Cleaved into: |
| | Phospholipase B-like 1 chain A; Phospholipase B-like 1 chain B; Phospholipase |
| | B-like 1 chain C] |
| Q8NHP8 | Putative phospholipase B-like 2 (EC 3.1.1.—) (76 kDa protein) (p76) (LAMA- |
| | like protein 2) (Lamina ancestor homolog 2) (Phospholipase B domain- |
| | containing protein 2) [Cleaved into: Putative phospholipase B-like 2 32 kDa |
| | form; Putative phospholipase B-like 2 45 kDa form] |
| Q9NQ66 | 1-phosphatidylinositol 4,5-bisphosphate phosphodiesterase beta-1 (EC 3.1.4.11) |
| | (PLC-154) (Phosphoinositide phospholipase C-beta-1) (Phospholipase C-I) |
| | (PLC-I) (Phospholipase C-beta-1) (PLC-beta-1) |
| Q8IV08 | Phospholipase D3 (PLD 3) (EC 3.1.4.4) (Choline phosphatase 3) (HindIII K4L |
| | homolog) (Hu-K4) (Phosphatidylcholine-hydrolyzing phospholipase D3) |
| Q96BZ4 | Phospholipase D4 (PLD 4) (EC 3.1.4.4) (Choline phosphatase 4) |
| | (Phosphatidylcholine-hydrolyzing phospholipase D4) |
| Q02809 | Procollagen-lysine,2-oxoglutarate 5-dioxygenase 1 (EC 1.14.11.4) (Lysyl |
| | hydroxylase 1) (LH1) |
| O00469 | Procollagen-lysine,2-oxoglutarate 5-dioxygenase 2 (EC 1.14.11.4) (Lysyl |
| | hydroxylase 2) (LH2) |
| O60568 | Procollagen-lysine,2-oxoglutarate 5-dioxygenase 3 (EC 1.14.11.4) (Lysyl |
| | hydroxylase 3) (LH3) |
| O14494 | Phospholipid phosphatase 1 (EC 3.1.3.4) (Lipid phosphate phosphohydrolase 1) |
| | (PAP2-alpha) (Phosphatidate phosphohydrolase type 2a) (Phosphatidic acid |
| | phosphatase 2a) (PAP-2a) (PAP2a) |
| O14495 | Phospholipid phosphatase 3 (EC 3.1.3.4) (Lipid phosphate phosphohydrolase 3) |
| | (PAP2-beta) (Phosphatidate phosphohydrolase type 2b) (Phosphatidic acid |
| | phosphatase 2b) (PAP-2b) (PAP2b) (Vascular endothelial growth factor and |
| | type I collagen-inducible protein) (VCIP) |
| P55058 | Phospholipid transfer protein (Lipid transfer protein II) |
| Q6UX71 | Plexin domain-containing protein 2 (Tumor endothelial marker 7-related |
| | protein) |
| Q9UIW2 | Plexin-A1 (Semaphorin receptor NOV) |
| O75051 | Plexin-A2 (Semaphorin receptor OCT) |
| Q9HCM2 | Plexin-A4 |
| O43157 | Plexin-B1 (Semaphorin receptor SEP) |
| O15031 | Plexin-B2 (MM1) |
| Q9ULL4 | Plexin-B3 |
| O60486 | Plexin-C1 (Virus-encoded semaphorin protein receptor) (CD antigen CD232) |
| Q9Y4D7 | Plexin-D1 |
| O00592 | Podocalyxin (GCTM-2 antigen) (Gp200) (Podocalyxin-like protein 1) (PC) |
| | (PCLP-1) |
| Q9NZ53 | Podocalyxin-like protein 2 (Endoglycan) |
| Q8NBL1 | Protein O-glucosyltransferase 1 (EC 2.4.1.—) (CAP10-like 46 kDa protein) |
| | (hCLP46) (KTEL motif-containing protein 1) (Myelodysplastic syndromes |
| | relative protein) (O-glucosyltransferase Rumi homolog) (hRumi) (Protein O- |
| | xylosyltransferase POGLUT1) (EC 2.4.2.26) |
| Q9H5K3 | Protein O-mannose kinase (POMK) (EC 2.7.1.183) (Protein kinase-like |
| | protei?gK196) (Sugen kinase 196) |
| Q9Y6A1 | Protein O-mannosyl-transferase 1 (EC 2.4.1.109) (Dolichyl-phosphate-mannose- |
| | -protein mannosyltransferase 1) |
| P27169 | Serum paraoxonase/arylesterase 1 (PON 1) (EC 3.1.1.2) (EC 3.1.1.81) (EC |
| | 3.1.8.1) (Aromatic esterase 1) (A-esterase 1) (K-45) (Serum |
| | aryldialkylphosphatase 1) |
| Q15165 | Serum paraoxonase/arylesterase 2 (PON 2) (EC 3.1.1.2) (EC 3.1.1.81) |
| | (Aromatic esterase 2) (A-esterase 2) (Serum aryldialkylphosphatase 2) |
| Q15063 | Periostin (PN) (Osteoblast-specific factor 2) (OSF-2) |
| Q9NUX5 | Protection of telomeres protein 1 (hPot1) (POT1-like telomere end-binding |
| | protein) |
| P0CG39 | POTE ankyrin domain family member J |
| Q13136 | Liprin-alpha-1 (LAR-interacting protein 1) (LIP-1) (Protein tyrosine |
| | phosphatase receptor type f polypeptide-interacting protein alpha-1) (PTPRF- |
| | interacting protein alpha-1) |
| 075335 | Liprin-alpha-4 (Protein tyrosine phosphatase receptor type f polypeptide- |
| | interacting protein alpha-4) (PTPRF-interacting protein alpha-4) |
| P62937 | Peptidyl-prolyl cis-trans isomerase A (PPIase A) (EC 5.2.1.8) (Cyclophilin A) |
| | (Cyclosporin A-binding protein) (Rotamase A) [Cleaved into: Peptidyl-prolyl |
| | cis-trans isomerase A, N-terminally processed] |
| P30405 | Peptidyl-prolyl cis-trans isomerase F, mitochondrial (PPIase F) (EC 5.2.1.8) |
| | (Cyclophilin D) (CyP-D) (CypD) (Cyclophilin F) (Mitochondrial cyclophilin) |
| | (CyP-M) (Rotamase F) |
| P49593 | Protein phosphatase 1F (EC 3.1.3.16) (Ca(2+)/calmodulin-dependent protein |
| | kinase phosphatase) (CaM-kinase phosphatase) (CaMKPase) (Partner of PIX 2) |
| | (Protein fem-2 homolog) (hFem-2) |
| Q08209 | Serine/threonine-protein phosphatase 2B catalytic subunit alpha isoform (EC |
| | 3.1.3.16) (CAM-PRP catalytic subunit) (Calmodulin-dependent calcineurin A |
| | subunit alpha isoform) |
| P16298 | Serine/threonine-protein phosphatase 2B catalytic subunit beta isoform (EC |
| | 3.1.3.16) (CAM-PRP catalytic subunit) (Calmodulin-dependent calcineurin A |
| | subunit beta isoform) |
| P50897 | Palmitoyl-protein thioesterase 1 (PPT-1) (EC 3.1.2.22) (Palmitoyl-protein |
| | hydrolase 1) |
| P42785 | Lysosomal Pro-X carboxypeptidase (EC 3.4.16.2) (Angiotensinase C) |
| | (Lysosomal carboxypeptidase C) (Proline carboxypeptidase) |
| | (Prolylcarboxypeptidase) (PRCP) |
| P51888 | Prolargin (Proline-arginine-rich end leucine-rich repeat protein) |
| Q70Z35 | Phosphatidylinositol 3,4,5-trisphosphate-dependent Rac exchanger 2 protein (P- |
| | Rex2) (PtdIns(3,4,5)-dependent Rac exchanger 2) (DEP domain-containing |
| | protein 2) |
| Q92954 | Proteoglycan 4 (Lubricin) (Megakaryocyte-stimulating factor) (Superficial zone |
| | proteoglycan) [Cleaved into: Proteoglycan 4 C-terminal part] |
| P14314 | Glucosidase 2 subunit beta (80K-H protein) (Glucosidase II subunit beta) |
| | (Protein kinase C substrate 60.1 kDa protein heavy chain) (PKCSH) |
| P04156 | Major prion protein (PrP) (ASCR) (PrP27-30) (PrP33-35C) (CD antigen |
| | CD230) |
| Q5JSZ5 | Protein PRRC2B (HLA-B-associated transcript 2-like 1) (Proline-rich coiled- |
| | coil protein 2B) |
| Q7Z6L0 | Proline-rich transmembrane protein 2 (Dispanin subfamily B member 3) |
| | (DSPB3) |
| O95084 | Serine protease 23 (EC 3.4.21.—) (Putative secreted protein Zsigl3) |
| P07602 | Prosaposin (Proactivator polypeptide) [Cleaved into: Saposin-A (Protein A); |
| | Saposin-B-Val; Saposin-B (Cerebroside sulfate activator) (CSAct) (Dispersin) |
| | (Sphingolipid activator protein 1) (SAP-1) (Sulfatide/GM1 activator); Saposin-C |
| | (A1 activator) (Co-beta-glucosidase) (Glucosylceramidase activator) |
| | (Sphingolipid activator protein 2) (SAP-2); Saposin-D (Component C) (Protein |
| | C)] |
| Q9Y617 | Phosphoserine aminotransferase (EC 2.6.1.52) (Phosphohydroxythreonine |
| | aminotransferase) (PSAT) |
| Q96NR3 | Patched domain-containing protein 1 |
| Q9BVG9 | Phosphatidylserine synthase 2 (PSS-2) (PtdSer synthase 2) (EC 2.7.8.29) |
| | (Serine-exchange enzyme II) |
| P41222 | Prostaglandin-H2 D-isomerase (EC 5.3.99.2) (Beta-trace protein) (Cerebrin-28) |
| | (Glutathione-independent PGD synthase) (Lipocalin-type prostaglandin-D |
| | synthase) (Prostaglandin-D2 synthase) (PGD2 synthase) (PGDS) (PGDS2) |
| Q9P2B2 | Prostaglandin F2 receptor negative regulator (CD9 partner 1) (CD9P-1) (Glu- |
| | Trp-Ile EWI motif-containing protein F) (EWI-F) (Prostaglandin F2-alpha |
| | receptor regulatory protein) (Prostaglandin F2-alpha receptor-associated protein) |
| | (CD antigen CD315) |
| P08575 | Receptor-type tyrosine-protein phosphatase C (EC 3.1.3.48) (Leukocyte |
| | common antigen) (L-CA) (T200) (CD antigen CD45) |
| P10586 | Receptor-type tyrosine-protein phosphatase F (EC 3.1.3.48) (Leukocyte |
| | common antigen related) (LAR) |
| Q12913 | Receptor-type tyrosine-protein phosphatase eta (Protein-tyrosine phosphatase |
| | eta) (R-PTP-eta) (EC 3.1.3.48) (Density-enhanced phosphatase 1) (DEP-1) |
| | (HPTP eta) (Protein-tyrosine phosphatase receptor type J) (R-PTP-J) (CD |
| | antigen CD148) |
| Q15262 | Receptor-type tyrosine-protein phosphatase kappa (Protein-tyrosine phosphatase |
| | kappa) (R-PTP-kappa) (EC 3.1.3.48) |
| P28827 | Receptor-type tyrosine-protein phosphatase mu (Protein-tyrosine phosphatase |
| | mu) (R-PTP-mu) (EC 3.1.3.48) |
| Q16849 | Receptor-type tyrosine-protein phosphatase-like N (R-PTP-N) (Islet cell antigen |
| | 512) (ICA 512) (Islet cell autoantigen 3) (PTP IA-2) [Cleaved into: ICA512-N- |
| | terminal fragment (ICA512-NTF); ICA512-transmembrane fragment (ICA512- |
| | TMF); ICA512-cleaved cytosolic fragment (ICA512-CCF)] |
| Q92932 | Receptor-type tyrosine-protein phosphatase N2 (R-PTP-N2) (EC 3.1.3.—) (EC |
| | 3.1.3.48) (Islet cell autoantigen-related protein) (IAR) (ICAAR) (Phogrin) |
| | [Cleaved into: IA-2beta60] |
| Q16827 | Receptor-type tyrosine-protein phosphatase O (R-PTP-O) (EC 3.1.3.48) |
| | (Glomerular epithelial protein 1) (Protein tyrosine phosphatase U2) (PTP-U2) |
| | (PTPase U2) |
| Q15256 | Receptor-type tyrosine-protein phosphatase R (R-PTP-R) (EC 3.1.3.48) (Ch- |
| | 1PTPase) (NC-PTPCOM1) (Protein-tyrosine phosphatase PCPTP1) |
| Q13332 | Receptor-type tyrosine-protein phosphatase S (R-PTP-S) (EC 3.1.3.48) |
| | (Receptor-type tyrosine-protein phosphatase sigma) (R-PTP-sigma) |
| O14522 | Receptor-type tyrosine-protein phosphatase T (R-PTP-T) (EC 3.1.3.48) |
| | (Receptor-type tyrosine-protein phosphatase rho) (RPTP-rho) |
| Q92729 | Receptor-type tyrosine-protein phosphatase U (R-PTP-U) (EC 3.1.3.48) |
| | (Pancreatic carcinoma phosphatase 2) (PCP-2) (Protein-tyrosine phosphatase J) |
| | (PTP-J) (hPTP-J) (Protein-tyrosine phosphatase pi) (PTP pi) (Protein-tyrosine |
| | phosphatase receptor omicron) (PTP-RO) (Receptor-type protein-tyrosine |
| | phosphatase psi) (R-PTP-psi) |
| P23471 | Receptor-type tyrosine-protein phosphatase zeta (R-PTP-zeta) (EC 3.1.3.48) |
| | (Protein-tyrosine phosphatase receptor type Z polypeptide 1) (Protein-tyrosine |
| | phosphatase receptor type Z polypeptide 2) (R-PTP-zeta-2) |
| P53801 | Pituitary tumor-transforming gene 1 protein-interacting protein (Pituitary tumor- |
| | transforming gene protein-binding factor) (PBF) (PTTG-binding factor) |
| P78406 | mRNA export factor (Rael protein homolog) (mRNA-associated protein mrnp |
| | 41) |
| Q86X10 | Ral GTPase-activating protein subunit beta (p170) |
| Q9UKM9 | RNA-binding protein Raly (Autoantigen p542) (Heterogeneous nuclear |
| | ribonucleoprotein C-like 2) (hnRNP core protein C-like 2) (hnRNP associated |
| | with lethal yellow protein homolog) |
| O60896 | Receptor activity-modifying protein 3 (Calcitonin-receptor-like receptor |
| | activity-modifying protein 3) (CRLR activity-modifying protein 3) |
| Q70E73 | Ras-associated and pleckstrin homology domains-containing protein 1 (RAPH1) |
| | (Amyotrophic lateral sclerosis 2 chromosomal region candidate gene 18 protein) |
| | (Amyotrophic lateral sclerosis 2 chromosomal region candidate gene 9 protein) |
| | (Lamellipodin) (Proline-rich EVH1 ligand 2) (PREL-2) (Protein RMO1) |
| Q16576 | Histone-binding protein RBBP7 (Histone acetyltransferase type B subunit 2) |
| | (Nucleosome-remodeling factor subunit RBAP46) (Retinoblastoma-binding |
| | protein 7) (RBBP-7) (Retinoblastoma-binding protein p46) |
| Q15293 | Reticulocalbin-1 |
| P78509 | Reelin (EC 3.4.21.—) |
| Q52LD8 | Raftlin-2 (Raft-linking protein 2) |
| Q96B86 | Repulsive guidance molecule A (RGM domain family member A) |
| Q6PJF5 | Inactive rhomboid protein 2 (iRhom2) (Rhomboid 5 homolog 2) (Rhomboid |
| | family member 2) (Rhomboid veinlet-like protein 5) (Rhomboid veinlet-like |
| | protein 6) |
| Q9UBD6 | Ammonium transporter Rh type C (Rh glycoprotein kidney) (Rhesus blood |
| | group family type C glycoprotein) (Rh family type C glycoprotein) (Rh type C |
| | glycoprotein) (Tumor-related protein DRC2) |
| O43567 | E3 ubiquitin-protein ligase RNF13 (EC 2.3.2.27) (RING finger protein 13) |
| | (RING-type E3 ubiquitin transferase RNF13) |
| Q63HN8 | E3 ubiquitin-protein ligase RNF213 (EC 2.3.2.27) (EC 3.6.4.—) (ALK lymphoma |
| | oligomerization partner on chromosome 17) (Mysterin) (RING finger protein |
| | 213) (RING-type E3 ubiquitin transferase RNF213) |
| P13489 | Ribonuclease inhibitor (Placental ribonuclease inhibitor) (Placental RNase |
| | inhibitor) (Ribonuclease/angiogenin inhibitor 1) (RAI) |
| Q9Y6N7 | Roundabout homolog 1 (Deleted in U twenty twenty) (H-Robo-1) |
| Q9HCK4 | Roundabout homolog 2 |
| Q9Y2J0 | Rabphilin-3A (Exophilin-1) |
| P04844 | Dolichyl-diphosphooligosaccharide--protein glycosyltransferase subunit 2 |
| | (Dolichyl-diphosphooligosaccharide--protein glycosyltransferase 63 kDa |
| | subunit) (RIBIIR) (Ribophorin II) (RPN-II) (Ribophorin-2) |
| P62263 | 40S ribosomal protein S14 (Small ribosomal subunit protein uS11) |
| Q92766 | Ras-responsive element-binding protein 1 (RREB-1) (Finger protein in nuclear |
| | bodies) (Raf-responsive zinc finger protein LZ321) (Zinc finger motif enhancer- |
| | binding protein 1) (Zep-1) |
| Q9BZR6 | Reticulon-4 receptor (Nogo receptor) (NgR) (Nogo-66 receptor) |
| Q86UN3 | Reticulon-4 receptor-like 2 (Nogo receptor-like 3) (Nogo-66 receptor homolog |
| | 1) (Nogo-66 receptor-related protein 2) (NgR2) |
| Q86VV8 | Rotatin |
| P21453 | Sphingosine 1-phosphate receptor 1 (SIP receptor 1) (S1P1) (Endothelial |
| | differentiation G-protein coupled receptor 1) (Sphingosine 1-phosphate receptor |
| | Edg-1) (S1P receptor Edg-1) (CD antigen CD363) |
| P35542 | Serum amyloid A-4 protein (Constitutively expressed serum amyloid A protein) |
| | (C-SAA) |
| Q9NTJ5 | Phosphatidylinositide phosphatase SAC1 (EC 3.1.3.—) (Suppressor of actin |
| | mutations 1-like protein) |
| Q8IVN8 | Somatomedin-B and thrombospondin type-1 domain-containing protein (RPE- |
| | spondin) |
| Q12770 | Sterol regulatory element-binding protein cleavage-activating protein (SCAP) |
| | (SREBP cleavage-activating protein) |
| Q6AZY7 | Scavenger receptor class A member 3 (Cellular stress response gene protein) |
| Q6ZMJ2 | Scavenger receptor class A member 5 (Scavenger receptor hlg) |
| Q8WTV0 | Scavenger receptor class B member 1 (SRB1) (CD36 and LIMPII analogous 1) |
| | (CLA-1) (CD36 antigen-like 1) (Collagen type I receptor, thrombospondin |
| | receptor-like 1) (SR-BI) (CD antigen CD36) |
| Q14108 | Lysosome membrane protein 2 (85 kDa lysosomal membrane sialoglycoprotein) |
| | (LGP85) (CD36 antigen-like 2) (Lysosome membrane protein II) (LIMP II) |
| | (Scavenger receptor class B member 2) (CD antigen CD36) |
| Q8NBX0 | Saccharopine dehydrogenase-like oxidoreductase (EC 1.—.—.—) |
| P35498 | Sodium channel protein type 1 subunit alpha (Sodium channel protein brain I |
| | subunit alpha) (Sodium channel protein type I subunit alpha) (Voltage-gated |
| | sodium channel subunit alpha Nav1.1) |
| Q07699 | Sodium channel subunit beta-1 |
| Q99250 | Sodium channel protein type 2 subunit alpha (HBSC II) (Sodium channel |
| | protein brain II subunit alpha) (Sodium channel protein type II subunit alpha) |
| | (Voltage-gated sodium channel subunit alpha Nav1.2) |
| O60939 | Sodium channel subunit beta-2 |
| Q8IWT1 | Sodium channel subunit beta-4 |
| Q9UQD0 | Sodium channel protein type 8 subunit alpha (Sodium channel protein type VIII |
| | subunit alpha) (Voltage-gated sodium channel subunit alpha Nav1.6) |
| O95487 | Protein transport protein Sec24B (SEC24-related protein B) |
| Q9UBV2 | Protein sel-1 homolog 1 (Suppressor of lin-12-like protein 1) (Sel-1L) |
| P49908 | Selenoprotein P (SeP) |
| Q9H3S1 | Semaphorin-4A (Semaphorin-B) (Sema B) |
| Q9NPR2 | Semaphorin-4B |
| Q9C0C4 | Semaphorin-4C |
| Q92854 | Semaphorin-4D (A8) (BB18) (GR3) (CD antigen CD100) |
| O75326 | Semaphorin-7A (CDw108) (JMH blood group antigen) (John-Milton-Hargen |
| | human blood group Ag) (Semaphorin-K1) (Serna K1) (Semaphorin-L) (Serna L) |
| | (CD antigen CD108) |
| Q9UH03 | Neuronal-specific septin-3 |
| Q99719 | Septin-5 (Cell division control-related protein 1) (CDCrel-1) (Peanut-like |
| | protein 1) |
| Q92599 | Septin-8 |
| Q8NC51 | Plasminogen activator inhibitor 1 RNA-binding protein (PAI1 RNA-binding |
| | protein 1) (PAI-RBP1) (SERPINE1 mRNA-binding protein 1) |
| Q86VE9 | Serine incorporator 5 |
| P01009 | Alpha-1-antitrypsin (Alpha-1 protease inhibitor) (Alpha-1-antiproteinase) |
| | (Serpin A1) [Cleaved into: Short peptide from AAT (SPAAT)] |
| Q9UK55 | Protein Z-dependent protease inhibitor (PZ-dependent protease inhibitor) (PZI) |
| | (Serpin A10) |
| P01011 | Alpha-1-antichymotrypsin (ACT) (Cell growth-inhibiting gene 24/25 protein) |
| | (Serpin A3) [Cleaved into: Alpha-1-antichymotrypsin His-Pro-less] |
| P05154 | Plasma serine protease inhibitor (Acrosomal serine protease inhibitor) |
| | (Plasminogen activator inhibitor 3) (PAI-3) (PAI3) (Protein C inhibitor) (PCI) |
| | (Serpin A5) |
| P08185 | Corticosteroid-binding globulin (CBG) (Serpin A6) (Transcortin) |
| P01008 | Antithrombin-III (ATIII) (Serpin C1) |
| P05546 | Heparin cofactor 2 (Heparin cofactor II) (HC-II) (Protease inhibitor leuserpin-2) |
| | (HLS2) (Serpin D1) |
| P07093 | Glia-derived nexin (GDN) (Peptidase inhibitor 7) (PI-7) (Protease nexin 1) (PN- |
| | 1) (Protease nexin I) (Serpin E2) |
| P36955 | Pigment epithelium-derived factor (PEDF) (Cell proliferation-inducing gene 35 |
| | protein) (EPC-1) (Serpin F1) |
| P05155 | Plasma protease C1 inhibitor (C1 Inh) (C1Inh) (C1 esterase inhibitor) (C1- |
| | inhibiting factor) (Serpin G1) |
| Q99574 | Neuroserpin (Peptidase inhibitor 12) (PI-12) (Serpin I1) |
| Q9BYH1 | Seizure 6-like protein |
| Q6UXD5 | Seizure 6-like protein 2 |
| Q16585 | Beta-sarcoglycan (Beta-SG) (43 kDa dystrophin-associated glycoprotein) |
| | (43DAG) (A3b) |
| Q92629 | Delta-sarcoglycan (Delta-SG) (35 kDa dystrophin-associated glycoprotein) |
| | (35DAG) |
| O43556 | Epsilon-sarcoglycan (Epsilon-SG) |
| P51688 | N-sulphoglucosamine sulphohydrolase (EC 3.10.1.1) (Sulfoglucosamine |
| | sulfamidase) (Sulphamidase) |
| Q6S5L8 | SHC-transforming protein 4 (Rai-like protein) (RaLP) (SHC-transforming |
| | protein D) (hShcD) (Src homology 2 domain-containing-transforming protein |
| | C4) (SH2 domain protein C4) |
| A6NL88 | Protein shisa-7 (Protein shisa-6-like) |
| B4DS77 | Protein shisa-9 |
| Q9HAT2 | Sialate O-acetylesterase (EC 3.1.1.53) (H-Lse) (Sialic acid-specific 9-O- |
| | acetylesterase) |
| Q9NXL6 | SID1 transmembrane family member 1 |
| Q8NBJ9 | SID1 transmembrane family member 2 |
| O15389 | Sialic acid-binding Ig-like lectin 5 (Siglec-5) (CD33 antigen-like 2) (Obesity- |
| | binding protein 2) (OB-BP2) (OB-binding protein 2) (CD antigen CD170) |
| P78324 | Tyrosine-protein phosphatase non-receptor type substrate 1 (SHP substrate 1) |
| | (SHPS-1) (Brain Ig-like molecule with tyrosine-based activation motifs) (Bit) |
| | (CD172 antigen-like family member A) (Inhibitory receptor SHPS-1) |
| | (Macrophage fusion receptor) (MyD-1 antigen) (Signal-regulatory protein alpha- |
| | 1) (Sirp-alpha-1) (Signal-regulatory protein alpha-2) (Sirp-alpha-2) (Signal- |
| | regulatory protein alpha-3) (Sirp-alpha-3) (p84) (CD antigen CD172a) |
| O00241 | Signal-regulatory protein beta-1 (SIRP-beta-1) (CD172 antigen-like family |
| | member B) (CD antigen CD172b) |
| Q5TFQ8 | Signal-regulatory protein beta-1 isoform 3 (SIRP-beta-1 isoform 3) |
| P49281 | Natural resistance-associated macrophage protein 2 (NRAMP 2) (Divalent |
| | cation transporter 1) (Divalent metal transporter 1) (DMT-1) (Solute carrier |
| | family 11 member 2) |
| P55011 | Solute carrier family 12 member 2 (Basolateral Na—K—Cl symporter) |
| | (Bumetanide-sensitive sodium-(potassium)-chloride cotransporter 1) |
| Q9H2X9 | Solute carrier family 12 member 5 (Electroneutral potassium-chloride |
| | cotransporter 2) (K—Cl cotransporter 2) (hKCC2) (Neuronal K—Cl cotransporter) |
| Q9BXP2 | Solute carrier family 12 member 9 (Cation-chloride cotransporter 6) (hCCC6) |
| | (Cation-chloride cotransporter-interacting protein 1) (CCC-interacting protein 1) |
| | (hCIP1) (Potassium-chloride transporter 9) (WO3.3) |
| Q86YT5 | Solute carrier family 13 member 5 (Na(+)/citrate cotransporter) (NaCT) |
| | (Sodium-coupled citrate transporter) (Sodium-dependent citrate transporter) |
| Q13336 | Urea transporter 1 (Solute carrier family 14 member 1) (Urea transporter, |
| | erythrocyte) |
| Q16348 | Solute carrier family 15 member 2 (Kidney H(+)/peptide cotransporter) |
| | (Oligopeptide transporter, kidney isoform) (Peptide transporter 2) |
| Q8N697 | Solute carrier family 15 member 4 (Peptide transporter 4) (Peptide/histidine |
| | transporter 1) (hPHT1) |
| Q9P2U8 | Vesicular glutamate transporter 2 (VGluT2) (Differentiation-associated BNPI) |
| | (Differentiation-associated Na(+)-dependent inorganic phosphate cotransporter) |
| | (Solute carrier family 17 member 6) |
| Q9P2U7 | Vesicular glutamate transporter 1 (VGluT1) (Brain-specific Na(+)-dependent |
| | inorganic phosphate cotransporter) (Solute carrier family 17 member 7) |
| P43004 | Excitatory amino acid transporter 2 (Glutamate/aspartate transporter II) |
| | (Sodium-dependent glutamate/aspartate transporter 2) (Solute carrier family 1 |
| | member 2) |
| P43003 | Excitatory amino acid transporter 1 (Sodium-dependent glutamate/aspartate |
| | transporter 1) (GLAST-1) (Solute carrier family 1 member 3) |
| P43007 | Neutral amino acid transporter A (Alanine/serine/cysteine/threonine transporter |
| | 1) (ASCT-1) (SATT) (Solute carrier family 1 member 4) |
| Q8WUG5 | Solute carrier family 22 member 17 (24p3 receptor) (24p3R) (Brain-type organic |
| | cation transporter) (Lipocalin-2 receptor) (Neutrophil gelatinase-associated |
| | lipocalin receptor) (NgalR) |
| A1A5C7 | Solute carrier family 22 member 23 |
| Q9H015 | Solute carrier family 22 member 4 (Ergothioneine transporter) (ET transporter) |
| | (Organic cation/carnitine transporter 1) |
| Q8NFF2 | Sodium/potassium/calcium exchanger 4 (Na(+)/K(+)/Ca(2+)-exchange protein |
| | 4) (Solute carrier family 24 member 4) |
| Q99808 | Equilibrative nucleoside transporter 1 (Equilibrative nitrobenzylmercaptopurine |
| | riboside-sensitive nucleoside transporter) (Equilibrative NBMPR-sensitive |
| | nucleoside transporter) (Nucleoside transporter, es-type) (Solute carrier family |
| | 29 member 1) |
| P11166 | Solute carrier family 2, facilitated glucose transporter member 1 (Glucose |
| | transporter type 1, erythrocyte/brain) (GLUT-1) (HepG2 glucose transporter) |
| Q96QE2 | Proton myo-inositol cotransporter (H(+)-myo-inositol cotransporter) (Hmit) |
| | (H(+)-myo-inositol symporter) (Solute carrier family 2 member 13) |
| P14672 | Solute carrier family 2, facilitated glucose transporter member 4 (Glucose |
| | transporter type 4, insulin-responsive) (GLUT-4) |
| Q9ULF5 | Zinc transporter ZIP10 (Solute carrier family 39 member 10) (Zrt- and Irt-like |
| | protein 10) (ZIP-10) |
| Q504Y0 | Zinc transporter ZIP12 (LIV-1 subfamily of ZIP zinc transporter 8) (LZT-Hs8) |
| | (Solute carrier family 39 member 12) (Zrt- and Irt-like protein 12) (ZIP-12) |
| Q13433 | Zinc transporter ZIP6 (Estrogen-regulated protein LIV-1) (Solute carrier family |
| | 39 member 6) (Zrt- and Irt-like protein 6) (ZIP-6) |
| P08195 | 4F2 cell-surface antigen heavy chain (4F2hc) (4F2 heavy chain antigen) |
| | (Lymphocyte activation antigen 4F2 large subunit) (Solute carrier family 3 |
| | member 2) (CD antigen CD98) |
| Q8NBI5 | Solute carrier family 43 member 3 (Protein FOAP-13) |
| Q8WWI5 | Choline transporter-like protein 1 (CDw92) (Solute carrier family 44 member 1) |
| | (CD antigen CD92) |
| Q8IWA5 | Choline transporter-like protein 2 (Solute carrier family 44 member 2) |
| Q96NT5 | Proton-coupled folate transporter (G21) (Heme carrier protein 1) (PCFT/HCP1) |
| | (Solute carrier family 46 member 1) |
| Q6U841 | Sodium-driven chloride bicarbonate exchanger (Solute carrier family 4 member |
| | 10) |
| Q2Y0W8 | Electroneutral sodium bicarbonate exchanger 1 (Electroneutral Na(+)-driven |
| | Cl—HCO3 exchanger) (Solute carrier family 4 member 8) (k-NBC3) |
| Q92911 | Sodium/iodide cotransporter (Na(+)/I(−) cotransporter) (Sodium-iodide |
| | symporter) (Na(+)/I(−) symporter) (Solute carrier family 5 member 5) |
| P48066 | Sodium- and chloride-dependent GABA transporter 3 (GAT-3) (Solute carrier |
| | family 6 member 11) |
| Q9NSD5 | Sodium- and chloride-dependent GABA transporter 2 (GAT-2) (Solute carrier |
| | family 6 member 13) |
| Q9H2J7 | Sodium-dependent neutral amino acid transporter B(0)AT2 (Sodium- and |
| | chloride-dependent neurotransmitter transporter NTT73) (Sodium-coupled |
| | branched-chain amino-acid transporter 1) (Solute carrier family 6 member 15) |
| | (Transporter v7-3) |
| Q9H1V8 | Sodium-dependent neutral amino acid transporter SLC6A17 (Sodium-dependent |
| | neurotransmitter transporter NTT4) (Solute carrier family 6 member 17) |
| Q99884 | Sodium-dependent proline transporter (Solute carrier family 6 member 7) |
| P30825 | High affinity cationic amino acid transporter 1 (CAT-1) (CAT1) (Ecotropic |
| | retroviral leukemia receptor homolog) (Ecotropic retrovirus receptor homolog) |
| | (Solute carrier family 7 member 1) (System Y+ basic amino acid transporter) |
| Q96T83 | Sodium/hydrogen exchanger 7 (Na(+)/H(+) exchanger 7) (NHE-7) (Solute |
| | carrier family 9 member 7) |
| Q8IVB4 | Sodium/hydrogen exchanger 9 (Na(+)/H(+) exchanger 9) (NHE-9) (Solute |
| | carrier family 9 member 9) |
| P0C7P3 | Protein SLFN14 [Cleaved into: C-terminally truncated SLFN14 |
| | endoribonuclease (EC 3.1.—.—) (Schlafen family member 14)] |
| O75093 | Slit homolog 1 protein (Slit-1) (Multiple epidermal growth factor-like domains |
| | protein 4) (Multiple EGF-like domains protein 4) |
| O94813 | Slit homolog 2 protein (Slit-2) [Cleaved into: Slit homolog 2 protein N-product; |
| | Slit homolog 2 protein C-product] |
| Q96PX8 | SLIT and NTRK-like protein 1 (Leucine-rich repeat-containing protein 12) |
| Q9H156 | SLIT and NTRK-like protein 2 |
| Q8IW52 | SLIT and NTRK-like protein 4 |
| Q12824 | SWI/SNF-related matrix-associated actin-dependent regulator of chromatin |
| | subfamily B member 1 (BRG1-associated factor 47) (BAF47) (Integrase |
| | interactor 1 protein) (SNF5 homolog) (hSNF5) |
| P17405 | Sphingomyelin phosphodiesterase (EC 3.1.4.12) (Acid sphingomyelinase) |
| | (aSMase) |
| Q8NB12 | Histone-lysine N-methyltransferase SMYD1 (EC 2.1.1.43) (SET and MYND |
| | domain-containing protein 1) |
| P62318 | Small nuclear ribonucleoprotein Sm D3 (Sm-D3) (snRNP core protein D3) |
| P00441 | Superoxide dismutase [Cu—Zn] (EC 1.15.1.1) (Superoxide dismutase 1) (hSod1) |
| P04179 | Superoxide dismutase [Mn], mitochondrial (EC 1.15.1.1) |
| P08294 | Extracellular superoxide dismutase [Cu—Zn] (EC-SOD) (EC 1.15.1.1) |
| O94875 | Sorbin and SH3 domain-containing protein 2 (Arg-binding protein 2) (ArgBP2) |
| | (Arg/Abl-interacting protein 2) (Sorbin) |
| Q8WY21 | VPS10 domain-containing receptor SorCS1 (hSorCS) |
| Q92673 | Sortilin-related receptor (Low-density lipoprotein receptor relative with 11 |
| | ligand-binding repeats) (LDLR relative with 11 ligand-binding repeats) (LR11) |
| | (SorLA-1) (Sorting protein-related receptor containing LDLR class A repeats) |
| | (SorLA) |
| Q99523 | Sortilin (100 kDa NT receptor) (Glycoprotein 95) (Gp95) (Neurotensin receptor |
| | 3) (NT3) (NTR3) |
| Q14515 | SPARC-like protein 1 (High endothelial venule protein) (Hevin) (MAST 9) |
| Q92563 | Testican-2 (SPARC/osteonectin, CWCV, and Kazal-like domains proteoglycan |
| | 2) |
| Q9BQ16 | Testican-3 (SPARC/osteonectin, CWCV, and Kazal-like domains proteoglycan |
| | 3) |
| Q9HCB6 | Spondin-1 (F-spondin) (Vascular smooth muscle cell growth-promoting factor) |
| Q8TCT8 | Signal peptide peptidase-like 2A (SPP-like 2A) (SPPL2a) (EC 3.4.23.—) |
| | (Intramembrane protease 3) (IMP-3) (Presenilin-like protein 2) |
| Q13813 | Spectrin alpha chain, non-erythrocytic 1 (Alpha-II spectrin) (Fodrin alpha chain) |
| | (Spectrin, non-erythroid alpha subunit) |
| Q01082 | Spectrin beta chain, non-erythrocytic 1 (Beta-II spectrin) (Fodrin beta chain) |
| | (Spectrin, non-erythroid beta chain 1) |
| P43307 | Translocon-associated protein subunit alpha (TRAP-alpha) (Signal sequence |
| | receptor subunit alpha) (SSR-alpha) |
| P43308 | Translocon-associated protein subunit beta (TRAP-beta) (Signal sequence |
| | receptor subunit beta) (SSR-beta) |
| O43173 | Sia-alpha-2,3-Gal-beta-1,4-GlcNAc-R: alpha 2,8-sialyltransferase (EC 2.4.99.—) |
| | (Alpha-2,8-sialyltransferase 8C) (Alpha-2,8-sialyltransferase III) (ST8 alpha-N- |
| | acetyl-neuraminide alpha-2,8-sialyltransferase 3) (Sialyltransferase 8C) (SIAT8- |
| | C) (Sialyltransferase St8Sia III) (ST8SiaIII) |
| Q9NY15 | Stabilin-1 (Fasciclin, EGF-like, laminin-type EGF-like and link domain- |
| | containing scavenger receptor 1) (FEEL-1) (MS-1 antigen) |
| Q687X5 | Metalloreductase STEAP4 (EC 1.16.1.—) (Six-transmembrane epithelial antigen |
| | of prostate 4) (SixTransMembrane protein of prostate 2) (Tumor necrosis factor, |
| | alpha-induced protein 9) |
| Q13586 | Stromal interaction molecule 1 |
| Q8WXE9 | Stonin-2 (Stoned B) |
| P08842 | Steryl-sulfatase (EC 3.1.6.2) (Arylsulfatase C) (ASC) (Steroid sulfatase) (Steryl- |
| | sulfate sulfohydrolase) |
| P46977 | Dolichyl-diphosphooligosaccharide--protein glycosyltransferase subunit STT3A |
| | (Oligosaccharyl transferase subunit STT3A) (STT3-A) (EC 2.4.99.18) (B5) |
| | (Integral membrane protein 1) (Transmembrane protein TMC) |
| Q8TCJ2 | Dolichyl-diphosphooligosaccharide--protein glycosyltransferase subunit STT3B |
| | (Oligosaccharyl transferase subunit STT3B) (STT3-B) (EC 2.4.99.18) (Source |
| | of immunodominant MHC-associated peptides homolog) |
| Q16623 | Syntaxin-1A (Neuron-specific antigen HPC-1) |
| P61266 | Syntaxin-1B (Syntaxin-1B1) (Syntaxin-1B2) |
| Q9Y2Z0 | Protei?GT1 homolog (Protein 40-6-3) (Sgt1) (Suppressor of G2 allele of SKP1 |
| | homolog) |
| Q8IWU6 | Extracellular sulfatase Sulf-1 (hSulf-1) (EC 3.1.6.—) |
| Q8IWU5 | Extracellular sulfatase Sulf-2 (hSulf-2) (EC 3.1.6.—) |
| O94901 | SUN domain-containing protein 1 (Protein unc-84 homolog A) (Sad1/unc-84 |
| | protein-like 1) |
| Q9UH99 | SUN domain-containing protein 2 (Protein unc-84 homolog B) (Rab5- |
| | interacting protein) (Rab5IP) (Sad1/unc-84 protein-like 2) |
| Q7L0J3 | Synaptic vesicle glycoprotein 2A |
| Q7L1I2 | Synaptic vesicle glycoprotein 2B |
| Q496J9 | Synaptic vesicle glycoprotein 2C |
| Q9BX26 | Synaptonemal complex protein 2 (SCP-2) (Synaptonemal complex lateral |
| | element protein) (hsSCP2) |
| O15061 | Synemin (Desmuslin) |
| Q8TBG9 | Synaptoporin |
| Q16563 | Synaptophysin-like protein 1 (Pantophysin) |
| Q86SS6 | Synaptotagmin-9 (Synaptotagmin IX) (SytIX) |
| Q6NUS6 | Tectonic-3 |
| Q9UKZ4 | Teneurin-1 (Ten-1) (Protein Odd Oz/ten-m homolog 1) (Tenascin-M1) (Ten-m1) |
| | (Teneurin transmembrane protein 1) [Cleaved into: Ten-1 intracellular domain |
| | (IDten-1) (Ten-1 ICD); Teneurin C-terminal-associated peptide (TCPA-1) (Ten- |
| | 1 extracellular domain) (Ten-1 ECD)] |
| Q9NT68 | Teneurin-2 (Ten-2) (Protein Odd Oz/ten-m homolog 2) (Tenascin-M2) (Ten-m2) |
| | (Teneurin transmembrane protein 2) [Cleaved into: Ten-2, soluble form; Ten-2 |
| | intracellular domain (Ten-2 ICD)] |
| Q9P273 | Teneurin-3 (Ten-3) (Protein Odd Oz/ten-m homolog 3) (Tenascin-M3) (Ten-m3) |
| | (Teneurin transmembrane protein 3) |
| Q6N022 | Teneurin-4 (Ten-4) (Protein Odd Oz/ten-m homolog 4) (Tenascin-M4) (Ten-m4) |
| | (Teneurin transmembrane protein 4) |
| Q6N021 | Methylcytosine dioxygenase TET2 (EC 1.14.11.n2) |
| P02787 | Serotransferrin (Transferrin) (Beta-1 metal-binding globulin) (Siderophilin) |
| P02786 | Transferrin receptor protein 1 (TR) (TfR) (TfR1) (Trfr) (T9) (p90) (CD antigen |
| | CD71) [Cleaved into: Transferrin receptor protein 1, serum form (sTfR)] |
| P01137 | Transforming growth factor beta-1 (TGF-beta-1) [Cleaved into: Latency- |
| | associated peptide (LAP)] |
| O43493 | Trans-Golgi network integral membrane protein 2 (TGN38 homolog) (TGN46) |
| | (TGN48) (Trans-Golgi network protein TGN51) |
| Q9UPZ6 | Thrombospondin type-1 domain-containing protein 7A |
| P04216 | Thy-1 membrane glycoprotein (CDw90) (Thy-1 antigen) (CD antigen CD90) |
| Q9GZM7 | Tubulointerstitial nephritis antigen-like (Glucocorticoid-inducible protein 5) |
| | (Oxidized LDL-responsive gene 2 protein) (OLRG-2) (Tubulointerstitial |
| | nephritis antigen-related protein) (TIN Ag-related protein) (TIN-Ag-RP) |
| P29401 | Transketolase (TK) (EC 2.2.1.1) |
| Q15399 | Toll-like receptor 1 (Toll/interleukin-1 receptor-like protein) (TIL) (CD antigen |
| | CD281) |
| O60603 | Toll-like receptor 2 (Toll/interleukin-1 receptor-like protein 4) (CD antigen |
| | CD282) |
| O15455 | Toll-like receptor 3 (CD antigen CD283) |
| Q9BX74 | TM2 domain-containing protein 1 (Amyloid-beta-binding protein) (hBBP) |
| Q9BRN9 | TM2 domain-containing protein 3 (Beta-amyloid-binding protein-like protein 2) |
| | (BBP-like protein 2) |
| Q9HD45 | Transmembrane 9 superfamily member 3 (EP70-P-iso) (SM-11044-binding |
| | protein) |
| Q7Z7H5 | Transmembrane emp24 domain-containing protein 4 (Endoplasmic reticulum |
| | stress-response protein 25) (ERS25) (GMP25iso) (Putative NF-kappa-B- |
| | activating protein 156) (p24 family protein alpha-3) (p24alpha3) |
| Q9BVK6 | Transmembrane emp24 domain-containing protein 9 (GMP25) (Glycoprotein |
| | 25L2) (p24 family protein alpha-2) (p24alpha2) (p25) |
| Q8IYR6 | Tomoregulin-1 (TR-1) (H7365) (Transmembrane protein with EGF-like and one |
| | follistatin-like domain) |
| Q9NUM4 | Transmembrane protein 106B |
| Q92545 | Transmembrane protein 131 (Protein RW1) |
| Q24JP5 | Transmembrane protein 132A (HSPA5-binding protein 1) |
| Q14DG7 | Transmembrane protein 132B |
| Q6IEE7 | Transmembrane protein 132E |
| Q8NBT3 | Transmembrane protein 145 |
| Q7Z7N9 | Transmembrane protein 179B |
| Q9P2C4 | Transmembrane protein 181 |
| Q9UHN6 | Cell surface hyaluronidase (EC 3.2.1.35) (Transmembrane protein 2) |
| Q9H813 | Transmembrane protein 206 |
| Q9H6L2 | Transmembrane protein 231 |
| Q9H330 | Transmembrane protein 245 (Protein CG-2) |
| Q86YD3 | Transmembrane protein 25 |
| Q4ZIN3 | Membralin (Transmembrane protein 259) |
| Q9NV96 | Cell cycle control protein 50A (P4-ATPase flippase complex beta subunit |
| | TMEM30A) (Transmembrane protein 30A) |
| Q8NBN3 | Transmembrane protein 87A |
| Q9P0T7 | Transmembrane protein 9 (Dermal papilla-derived protein 4) |
| P28289 | Tropomodulin-1 (Erythrocyte tropomodulin) (E-Tmod) |
| Q9H3S3 | Transmembrane protease serine 5 (EC 3.4.21.—) (Spinesin) |
| Q5T4D3 | Transmembrane and TPR repeat-containing protein 4 |
| Q96JJ7 | Protein disulfide-isomerase TMX3 (EC 5.3.4.1) (Thioredoxin domain- |
| | containing protein 10) (Thioredoxin-related transmembrane protein 3) |
| P24821 | Tenascin (TN) (Cytotactin) (GMEM) (GP 150-225) (Glioma-associated- |
| | extracellular matrix antigen) (Hexabrachion) (JI) (Myotendinous antigen) |
| | (Neuronectin) (Tenascin-C) (TN-C) |
| O75509 | Tumor necrosis factor receptor superfamily member 21 (Death receptor 6) (CD |
| | antigen CD358) |
| O75888 | Tumor necrosis factor ligand superfamily member 13 (A proliferation-inducing |
| | ligand) (APRIL) (TNF- and APOL-related leukocyte expressed ligand 2) |
| | (TALL-2) (TNF-related death ligand 1) (TRDL-1) (CD antigen CD256) |
| Q92752 | Tenascin-R (TN-R) (Janusin) (Restrictin) |
| Q8NDV7 | Trinucleotide repeat-containing gene 6A protein (CAG repeat protein 26) |
| | (EMSY interactor protein) (GW 182 autoantigen) (Protein GW1) (Glycine- |
| | tryptophan protein of 182 kDa) |
| P22105 | Tenascin-X (TN-X) (Hexabrachion-like protein) |
| Q8NFQ8 | Torsin-1A-interacting protein 2 (Lumenal domain-like LAP1) |
| Q9H497 | Torsin-3A (ATP-dependent interferon-responsive protein) (Torsin family 3 |
| | member A) |
| Q13641 | Trophoblast glycoprotein (5T4 oncofetal antigen) (5T4 oncofetal trophoblast |
| | glycoprotein) (5T4 oncotrophoblast glycoprotein) (M6P1) (Wnt-activated |
| | inhibitory factor 1) (WAIF1) |
| O14773 | Tripeptidyl-peptidase 1 (TPP-1) (EC 3.4.14.9) (Cell growth-inhibiting gene 1 |
| | protein) (Lysosomal pepstatin-insensitive protease) (LPIC) (Tripeptidyl |
| | aminopeptidase) (Tripeptidyl-peptidase I) (TPP-I) |
| P13693 | Translationally-controlled tumor protein (TCTP) (Fortilin) (Histamine-releasing |
| | factor) (HRF) (p23) |
| Q9UKU6 | Thyrotropin-releasing hormone-degrading ectoenzyme (TRH-DE) (TRH- |
| | degrading ectoenzyme) (EC 3.4.19.6) (Pyroglutamyl-peptidase II) (PAP-II) |
| | (TRH-specific aminopeptidase) (Thyroliberinase) |
| Q7L0X0 | TLR4 interactor with leucine rich repeats (Leucine-rich repeat-containing |
| | protein KIAA0644) |
| Q9C040 | Tripartite motif-containing protein 2 (EC 2.3.2.27) (E3 ubiquitin-protein ligase |
| | TRIM2) (RING finger protein 86) (RING-type E3 ubiquitin transferase TRIM2) |
| O75962 | Triple functional domain protein (EC 2.7.11.1) (PTPRF-interacting protein) |
| Q9HCF6 | Transient receptor potential cation channel subfamily M member 3 (Long |
| | transient receptor potential channel 3) (LTrpC-3) (LTrpC3) (Melastatin-2) |
| | (MLSN2) |
| Q8NER1 | Transient receptor potential cation channel subfamily V member 1 (TrpV1) |
| | (Capsaicin receptor) (Osm-9-like TRP channel 1) (OTRPC1) (Vanilloid receptor |
| | 1) |
| O95857 | Tetraspanin-13 (Tspan-13) (Tetraspan NET-6) (Transmembrane 4 superfamily |
| | member 13) |
| O95858 | Tetraspanin-15 (Tspan-15) (Tetraspan NET-7) (Transmembrane 4 superfamily |
| | member 15) |
| O60637 | Tetraspanin-3 (Tspan-3) (Tetraspanin TM4-A) (Transmembrane 4 superfamily |
| | member 8) |
| Q12999 | Tetraspanin-31 (Tspan-31) (Sarcoma-amplified sequence) |
| Q86UF1 | Tetraspanin-33 (Tspan-33) (Penumbra) (hPen) (Proerythroblast new membrane) |
| P41732 | Tetraspanin-7 (Tspan-7) (Cell surface glycoprotein A15) (Membrane component |
| | chromosome X surface marker 1) (T-cell acute lymphoblastic leukemia- |
| | associated antigen 1) (TALLA-1) (Transmembrane 4 superfamily member 2) |
| | (CD antigen CD231) |
| P19075 | Tetraspanin-8 (Tspan-8) (Transmembrane 4 superfamily member 3) (Tumor- |
| | associated antigen CO-029) |
| Q8WZ42 | Titin (EC 2.7.11.1) (Connectin) (Rhabdomyosarcoma antigen MU-RMS-40.14) |
| Q9H313 | Protein tweety homolog 1 (hTTY1) |
| Q9C0H2 | Protein tweety homolog 3 (hTTY3) |
| Q71U36 | Tubulin alpha-1A chain (Alpha-tubulin 3) (Tubulin B-alpha-1) (Tubulin alpha-3 |
| | chain) [Cleaved into: Detyrosinated tubulin alpha-1 A chain] |
| Q9BQE3 | Tubulin alpha-1C chain (Alpha-tubulin 6) (Tubulin alpha-6 chain) [Cleaved |
| | into: Detyrosinated tubulin alpha-1C chain] |
| P68366 | Tubulin alpha-4A chain (Alpha-tubulin 1) (Testis-specific alpha-tubulin) |
| | (Tubulin H2-alpha) (Tubulin alpha-1 chain) |
| P07437 | Tubulin beta chain (Tubulin beta-5 chain) |
| Q13885 | Tubulin beta-2A chain (Tubulin beta class IIa) |
| Q13509 | Tubulin beta-3 chain (Tubulin beta-4 chain) (Tubulin beta-III) |
| P04350 | Tubulin beta-4A chain (Tubulin 5 beta) (Tubulin beta-4 chain) |
| Q3ZCM7 | Tubulin beta-8 chain (Tubulin beta 8 class VIII) |
| Q96J42 | Thioredoxin domain-containing protein 15 |
| Q06418 | Tyrosine-protein kinase receptor TYRO3 (EC 2.7.10.1) (Tyrosine-protein kinase |
| | BYK) (Tyrosine-protein kinase DTK) (Tyrosine-protein kinase RSE) (Tyrosine- |
| | protein kinase SKY) (Tyrosine-protein kinase TIF) |
| P22314 | Ubiquitin-like modifier-activating enzyme 1 (EC 6.2.1.45) (Protein A1S9) |
| | (Ubiquitin-activating enzyme E1) |
| Q92575 | UBX domain-containing protein 4 (Erasin) (UBX domain-containing protein 2) |
| P09936 | Ubiquitin carboxyl-terminal hydrolase isozyme L1 (UCH-L1) (EC 3.4.19.12) |
| | (EC 6.—.—.—) (Neuron cytoplasmic protein 9.5) (PGP 9.5) (PGP9.5) (Ubiquitin |
| | thioesterase L1) |
| Q9NYU2 | UDP-glucose: glycoprotein glucosyltransferase 1 (UGT1) (hUGT1) (EC 2.4.1.—) |
| | (UDP--Glc: glycoprotein glucosyltransferase) (UDP-glucose ceramide |
| | glucosyltransferase-like 1) |
| Q16880 | 2-hydroxyacylsphingosine 1-beta-galactosyltransferase (EC 2.4.1.47) (Ceramide |
| | UDP-galactosyltransferase) (Cerebroside synthase) (UDP-galactose-ceramide |
| | galactosyltransferase) |
| Q6ZN44 | Netrin receptor UNC5A (Protein unc-5 homolog 1) (Protein unc-5 homolog A) |
| O95185 | Netrin receptor UNC5C (Protein unc-5 homolog 3) (Protein unc-5 homolog C) |
| P31930 | Cytochrome b-c1 complex subunit 1, mitochondrial (Complex III subunit 1) |
| | (Core protein I) (Ubiquinol-cytochrome-c reductase complex core protein 1) |
| P22695 | Cytochrome b-c1 complex subunit 2, mitochondrial (Complex III subunit 2) |
| | (Core protein II) (Ubiquinol-cytochrome-c reductase complex core protein 2) |
| P54578 | Ubiquitin carboxyl-terminal hydrolase 14 (EC 3.4.19.12) (Deubiquitinating |
| | enzyme 14) (Ubiquitin thioesterase 14) (Ubiquitin-specific-processing protease |
| | 14) |
| P45974 | Ubiquitin carboxyl-terminal hydrolase 5 (EC 3.4.19.12) (Deubiquitinating |
| | enzyme 5) (Isopeptidase T) (Ubiquitin thioesterase 5) (Ubiquitin-specific- |
| | processing protease 5) |
| Q6EMK4 | Vasorin (Protein slit-like 2) |
| Q9HCJ6 | Synaptic vesicle membrane protein VAT-1 homolog-like (EC 1.—.—.—) |
| P19320 | Vascular cell adhesion protein 1 (V-CAM 1) (VCAM-1) (INCAM-100) (CD |
| | antigen CD106) |
| P13611 | Versican core protein (Chondroitin sulfate proteoglycan core protein 2) |
| | (Chondroitin sulfate proteoglycan 2) (Glial hyaluronate-binding protein) |
| | (GHAP) (Large fibroblast proteoglycan) (PG-M) |
| P45880 | Voltage-dependent anion-selective channel protein 2 (VDAC-2) (hVDAC2) |
| | (Outer mitochondrial membrane protein porin 2) |
| Q6UXI7 | Vitrin |
| Q8TAG5 | V-set and transmembrane domain-containing protein 2A |
| P04004 | Vitronectin (VN) (S-protein) (Serum-spreading factor) (V75) [Cleaved into: |
| | Vitronectin V65 subunit; Vitronectin V10 subunit; Somatomedin-B] |
| Q6PCB0 | von Willebrand factor A domain-containing protein 1 |
| P04275 | von Willebrand factor (vWF) [Cleaved into: von Willebrand antigen 2 (von |
| | Willebrand antigen II)] |
| O75083 | WD repeat-containing protein 1 (Actin-interacting protein 1) (AIP1) (NORI-1) |
| Q8TAF3 | WD repeat-containing protein 48 (USP1-associated factor 1) (WD repeat |
| | endosomal protein) (p80) |
| O76024 | Wolframin |
| Q93097 | Protein Wnt-2b (Protein Wnt-13) |
| O43895 | Xaa-Pro aminopeptidase 2 (EC 3.4.11.9) (Aminoacylproline aminopeptidase) |
| | (Membrane-bound aminopeptidase P) (Membrane-bound APP) (Membrane- |
| | bound AmP) (mAmP) (X-Pro aminopeptidase 2) |
| P13010 | X-ray repair cross-complementing protein 5 (EC 3.6.4.—) (86 kDa subunit of Ku |
| | antigen) (ATP-dependent DNA helicase 2 subunit 2) (ATP-dependent DNA |
| | helicase II 80 kDa subunit) (CTC box-binding factor 85 kDa subunit) (CTC85) |
| | (CTCBF) (DNA repair protein XRCC5) (Ku80) (Ku86) (Lupus Ku autoantigen |
| | protein p86) (Nuclear factor IV) (Thyroid-lupus autoantigen) (TLAA) (X-ray |
| | repair complementing defective repair in Chinese hamster cells 5 (double- |
| | strand-break rejoining)) |
| Q9Y2K1 | Zinc finger and BTB domain-containing protein 1 |
| Q7Z3T8 | Zinc finger FYVE domain-containing protein 16 (Endofin) (Endosome- |
| | associated FYVE domain protein) |
| Q9C0D4 | Zinc finger protein 518B |
| O15015 | Zinc finger protein 646 |
| Q96N22 | Zinc finger protein 681 |

A further embodiment includes methods of producing any of the recombinant fusion proteins described herein. The method includes a step of culturing a host cell (e.g., HEK293), including a vector which comprises a nucleic acid encoding the recombinant fusion protein under conditions permitting the production of the recombinant fusion protein. Exemplary vectors include (i) SEQ ID NO. 35, SEQ ID NO. 36, or SEQ ID NO. 34, which encode Grp94 in which several residues are deleted from the Pre-N domain, and (ii) the nucleic acid sequence encoding the protein fused to the Grp94. Suitable vectors also include (i) SEQ ID NO. 48, SEQ ID NO. 49, SEQ ID NO. 50, or SEQ ID NO. 51, which encode Grp94 in which several residues are substituted with alanine, and (ii) the nucleic acid sequence encoding the protein fused to the Grp94. It should be appreciated that vectors may include DNA sequences having about 95%, about 96%, about 97%, about 98%, or about 99% sequence homology with SEQ ID NO. 35, SEQ ID NO. 36, SEQ ID NO. 34, SEQ ID NO. 48, SEQ ID NO. 49, SEQ ID NO. 50, or SEQ ID NO. 51. The recombinant fusion protein may also be recovered.

In another embodiment, a method of glycosylating a protein includes the steps of expressing any of the recombinant fusion proteins described herein in a host cell. When the recombinant fusion protein includes a cleavable linker (e.g., a protease cleavable linker), the method may include a step of cleaving the protein from the wild type or recombinant Grp94. The recombinant fusion protein may also include a tag, such as a histidine tag, a flag tag, or a biotin tag that facilitates isolation of the recombinant fusion protein or the tagged portion thereof. In some embodiments, the protein is glycosylated at a glycosylation sequon, such as Asn-X-Ser or Asn-X-Thr (X is any amino acid except proline). In a further embodiment, the recombinant fusion protein is coexpressed with oligosaccharyltransferase (OST) complex.

### Recombinant Expression Vectors and Host Cells

Another aspect of the invention pertains to vectors, preferably expression vectors, containing a nucleic acid encoding a Grp94 protein (or a portion thereof), or a Grp94 fusion protein. Grp94 proteins, functional fragments thereof, and fusion proteins can be produced from wild type Grp94 sequences found in animals (e.g., human, canine, mouse, rat, cow, sheep, goat and birds), insect cells, plants, yeast or bacteria, as known in the art using the methods described herein. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., Grp94 proteins, Grp94 protein fragments, Grp94 fusion proteins, and the like).

The recombinant expression vectors of the invention can be designed for expression of Grp94 proteins and fusions in prokaryotic or eukaryotic cells. For example, Grp94 proteins and fusions can be expressed in bacterial cells such as E. coli, insect cells (using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three (3) purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.), which fuse glutathione S-transferase (GST), maltose E binding protein, or protein A, respectively, to the target recombinant protein.

Purified glycosylated proteins or Grp94 fusion proteins can be utilized in protein activity assays, (e.g., direct assays or competitive assays described in detail herein), or to generate antibodies specific for glycosylated proteins or Grp94 fusion proteins, for example.

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1 :268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264, 166).

Developmentally-regulated promoters are also encompassed, for example the murine hox promoters (Kessel and Gruss (1990) Science 249:374-379) and the a-fetoprotein promoter (Campes and Tilghman (1989) Genes Dev. 3:537-546).

Suitable mammalian cells that can be used to obtain Grp94 proteins, fusion proteins, or functional fragments include primary cell cultures derived from a mammal at any stage of development or maturity. Mammalian cells also include cells of mammalian origin that have been transformed to divide for an unlimited number of generation, such as human embryonic kidney line (e.g., HEK293), human fibrosarcoma cell line (e.g., HT1080), human cervical carcinoma cells (HeLa), human lung cells (W138), human liver cells (Hep G2), human retinoblasts, BALB/c mouse myeloma line, COS-7, baby hamster kidney cells (e.g., BHK), Chinese hamster ovary cells (e.g., CHO+/−DHFR), mouse Sertoli cells (TM4), rat liver cells (BRL 3A), mouse mammary tumor (e.g., MMT-060562), TRI cells; MRC 5 cells, FS4 cells, monkey kidney cells (e.g., CV1, VERO-76), and canine kidney cells (e.g., MDCK). Different host cells can be chosen to ensure its capacity to modify and process Grp94 proteins, fusion proteins, or functional fragments thereof.

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to Grp94 mRNA. Regulatory sequences operatively linked to a nucleic acid cloned in the antisense orientation can be chosen which direct the continuous expression of the antisense RNA molecule in a variety of cell types; for instance, viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al, Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the terms as used herein.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed, Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an Grp94 protein or fusion protein, or can be introduced on a separate vector. Cells stably transfected with the introduced nucleic acid can be identified by drug selection (e.g., cells that have incorporated the selectable marker gene will survive, while the other cells die).

Suitable host cells may be mammalian. Exemplary host cells include: human embryonic kidney line (e.g., HEK293), human fibrosarcoma cell line (e.g., HT1080), human cervical carcinoma cells (HeLa), human lung cells (W138), human liver cells (Hep G2), human retinoblasts, BALB/c mouse myeloma line, COS-7, baby hamster kidney cells (e.g., BHK), Chinese hamster ovary cells (e.g., CHO+/−DHFR), mouse Sertoli cells (TM4), rat liver cells (BRL 3A), mouse mammary tumor (e.g., MMT-060562), TRI cells; MRC 5 cells, FS4 cells, monkey kidney cells (e.g., CV1, VERO-76), and canine kidney cells (e.g., MDCK).

In further embodiments, the glycosylated protein produced by the methods described herein is a drug target and may be used as substrates for drug screens. In another embodiment, the glycosylated protein is a therapeutic protein that may be formulated as a pharmaceutical composition with a pharmaceutically acceptable excipient and optionally included in a kit with reagents, supplies, and/or instructions for use. In another embodiment, the glycosylated protein is an antigen that can be used to produce antibodies for use in assays, protein purification, or as therapeutic antibodies.

### Assays

In some embodiments, recombinant Grp94 proteins (including, but not limited to chimeric proteins) disclosed herein and/or antibodies directed to such proteins may be developed using assays described herein. In some embodiments, recombinant proteins (including, but not limited to chimeric proteins) disclosed herein and/or antibodies directed to such proteins may be used in assays to develop other recombinant proteins and/or antibodies of the present invention.

Binding Assays. In some embodiments, the present invention provides binding assays. As used herein, the term “binding assay” refers to an assay used to assess the ability of two or more factors to associate. Such assays may assess the ability of a desired antigen to bind a desired antibody and then use one or more detection methods to detect binding. Binding assays of the invention may include, but are not limited to surface Plasmon resonance-based assays, ELISAs and FACS-based assays. Binding assays of the invention may comprise the use of one or more recombinant proteins described herein, including, but not limited to any glycosylated proteins, any chimeric proteins, any cofactors and any modules, combinations or fragments thereof.

Cell-Based Assays. In some embodiments, the present invention provides cell-based assays. As used herein, the term “cell-based assay” refers to an assay comprising at least one aspect that involves the use of a living cell or cell culture. In some embodiments, these may be useful for assessing the modulation of protein activity based on the degree of glycosylation. Cell-based assays of the present invention may comprise expression cells and/or responsive cells. Expression cells, as referred to herein, are cells that express one or more factors being analyzed in a particular assay. Such expression may be natural or may be the result of transfection and/or transduction of a foreign gene. In some embodiments, expression of one or more factors by expression cells may be enhanced or suppressed by the addition of one or more exogenous factors. In some embodiments, expression cells may comprise cell lines (e.g. HEK293 cells, CHO cells, TMLC cells, 293T/17 cells, Hs68 cells, CCD1112sk cells, HFF-1 cells, Keloid fibroblasts or Sw-480 cells.) In some embodiments, cell lines comprising expression cells may express one or more recombinant proteins of the present invention (e.g. naturally and/or through transfection, stable transfection, and/or transduction).

### Antibodies

In one embodiment, a method for manufacturing an antibody, or an antigen-binding portion thereof, includes the steps of: (i) providing a glycosylated antigen produced by the methods described herein; and (ii) selecting a pool of antibodies, or antigen-binding portion thereof, for the ability to bind the glycosylated antigen of step (i). In some embodiments, the antibody, or antigen-binding portion thereof is a human or humanized antibody or antigen-binding portion thereof. In some embodiments, the method further includes a step of immunizing a host animal with the antigen of step (i). Optionally, the method may include collecting lymphocytes from the host animal that bind the antigen of step (i).

In one embodiment, step (ii) includes screening a library, e.g., an antibody display library. In another embodiment, step (ii) includes screening an antibody fragment display library, e.g. a library of Fab fragments, and/or single-chain variable fragments (scFvs). The antibody, or antigen-binding portion thereof, may be an IgA, IgD, IgE, IgG or IgM antibody.

In one embodiment, a method for manufacturing a pharmaceutical composition comprising an antibody, or an antigen-binding portion thereof, includes the steps of: (i) providing a glycosylated antigen produced by the methods described herein; (ii) selecting a pool of antibodies, or antigen-binding portion thereof, for the ability to bind the glycosylated antigen of step (i); (iii) selecting from the pool of antibodies, or antigen-binding portions thereof, an antibody, or an antigen-binding portion thereof that exhibits a desired effect; and, (iv) formulating the antibody or antigen-binding portion thereof from step (iii) into a pharmaceutical composition, wherein the antibody, or antigen-binding portion thereof is a human or humanized antibody or antigen-binding portion thereof.

### EXAMPLES

Experimental procedures, including cloning and construct generation, are now discussed with respect to a specific working example. All constructs used in these studies were derived from canine Grp94 (UniProt P41148), which is −98% identical to human Grp94. For mammalian expression, full-length and Pre-N truncated Grp94 genes were generated by PCR and inserted into the pTT5 vector between NotI and BamHI restriction sites. For bacterial expression, Grp94 was cloned into the NdeI and BamHI sites of the pET15b vector. All constructs were confirmed by sequencing.

Protein expression and purification in mammalian cells were carried out as follows: Mammalian cells were cultured in Freestyle293 media (Thermo Fisher) in a 37° C. incubator with 5% humidified CO2 and shaken at 140 rpm. Cells were transfected using a 4 to 1 ratio of branched PEI (Sigma) to vector and cultured for 24-72 hours as indicated before harvesting.

For purification of Grp94 for biochemical and ATPase analyses, cultures were transfected with 0.7-0.8 μg DNA per million cells and incubated for 72 hours. Cells were harvested by centrifugation at 500×g for 3 minutes, the culture supernatant was removed, cells were washed in ice-cold PBS, and then lysed by incubating in buffer consisting of 50 mM Tris-HCl (pH 8.0), 350 mM NaCl, 20 mM imidazole, and 0.5% CHAPS on ice for 30 minutes with periodic mixing. Lysis debris was cleared by centrifugation at 10,000×g for 5 minutes at 4° C. Ni-NTA agarose (Qiagen) was then added in batch to the lysate and allowed to capture His-tagged proteins overnight with nutation at 4° C. After washing with lysis buffer containing decreasing amounts of detergent, protein was stepwise eluted from the resin with buffer consisting of 50 mM Tris-HCl (pH 8.0), 150 mM NaCl, 350 mM imidazole, and 1 mM β-me. Eluted proteins were then injected onto a Superdex 200 16/60 (GE Healthcare Life Sciences) size-exclusion column equilibrated in 40 mM HEPES-KOH (pH 7.6), 150 mM KCl, and 5 mM MgCl2. Peak fractions were pooled and concentrated before flash freezing in liquid nitrogen.

Protein expression and purification in E. coli was carried out as follows. Proteins were expressed and purified as previously described (Dollins et al., 2007b) (Huck et al., 2017b). Briefly, constructs were expressed in BL21-Star (DE3) (Invitrogen) as N-terminal hexa-histidine fusion proteins. Cultures were typically grown at 37° C. and induced at mid-log with IPTG to a final concentration of 0.5-1.0 mM. The protein purification for all constructs consisted of Ni-affinity, Q-Sepharose anion exchange, and gel filtration purification steps. Protein fractions were concentrated between 10-30 mg/ml, aliquoted, and flash-frozen in liquid nitrogen.

For western blot analysis of mammalian cell lysates, cells were harvested by centrifugation at 500×g for 3 minutes following 24 or 48 hours of expression. The culture supernatant was removed, cells were washed in ice-cold PBS, and then lysed by incubating in buffer consisting of 50 mM Tris-HCl (pH 8.0), 150 mM NaCl, 1% Triton X-100, 0.5% Sodium deoxycholate, and 0.1% SDS on ice for 30 minutes with periodic mixing. Lysis debris was cleared by centrifugation at 10,000×g for 15 minutes at 4° C. Prior to western blot, samples were quantified using the Pierce BCA protein assay kit. In general, samples were mixed with 5× Laemmli buffer and run on 7.5% or 4-15% SDS-PAGE gels as indicated. Gels were run at 100 V for 2-2.5 hours using Tris-glycine SDS running buffer and transferred onto Bio-Rad Immun-Blot PVDF membranes using a BioRad Semi-Dry apparatus at 12 V for 70 minutes in transfer buffer. Membranes were blocked for 1 hour using 5% w/v non-fat milk in TBS-T (Tris-buffered saline with 1% Tween-20) and then incubated with primary antibodies diluted in 2% milk in TBS-T overnight at 4° C. or for 2 hours at room temperature. All primary antibodies were used at a final dilution of 1:1,000 or 1:2000. Membranes were then washed with 1×TBS-T and incubated with HRP-conjugated secondary antibodies (from) diluted 1:10,000 in 2% milk in TBS-T for 1 hour at room temperature. Membranes were washed in 1×TBS-T and chemiluminescent substrate (Thermo-Fisher SuperSignal West Pico) was added. Signal was captured using a Bio-Rad Chemi-Doc imaging system. Densitometry was performed using ImageJ.

Purified Grp94 samples were partially de-glycosylated with Endo-H (New England Biolabs) prior to mass spectrometry analysis. 150 μg of protein were denatured by adding 1× denaturing buffer and heating to 99° C. for 8 minutes followed by cooling on ice. De-glycosylation was achieved by adding 1× Glycobuffer-3 and 5 μL of Endo-H (2500 units) and incubating at 37° C. for 2 hours. De-glycosylation was confirmed by SDS-PAGE. Endo-H treated Grp94 was then precipitated using TCA to remove detergents, salts, and sugars. The mixture was incubated on ice for 30 minutes and then spun at 14,000×g for 5 minutes to pellet the precipitant. The supernatant was removed and the pellet was washed twice with 200 μL of ice-cold acetone. The pellet was dried by heating the tube to 95° C. using a heat block for 1-2 minutes. The dry pellet was re-suspended in 20 μL of 8 M urea (in 0.1 M Tris-HCl pH 8.5) and subsequently diluted to 2 M urea. Tris(2-carboxyethyl)phosphine (TCEP) was added to 5 mM final and the samples were heated to 95° C. for 10 minutes to reduce disulfide bonds. Freshly prepared iodoacetamide was then added for a final concentration of 10 mM and incubated at room temperature for 30 minutes to alkylate free cysteines. The samples were then buffer exchanged into 100 mM NH4HCO3 using a 7-kDa MWCO Zeba desalting column (Thermo Scientific) according to the manufacturer's protocol. Finally, the samples were proteolyzed by adding 2.25 μg of Trypsin Gold (Promega) or 2.3 μg of Endoproteinase LysC (New England Biolabs) and incubating at 37° C. for ˜40 hours with shaking. Proteolysis was confirmed by SDS-PAGE.

Mass spectrometry (MS) analysis was performed using an Orbitrap-XL spectrometer (Thermo) equipped with a nano-LC column (PepMap C18 2 μm; 75 μm x150 mm, Thermo) and ESI (electrospray ionization) source. In the Orbitrap-XL runs, the mobile phases were, A: water and B: acetonitrile (CH3CN), both containing 0.1% (v/v) formic acid. Data were acquired over 70 min using the following gradient: linear at a flow rate of 300 nL/min: (i) increase from 0% to 10% B over 5 min; (ii) 10% to 45% B over 60 min; (iii) 45% to 100% B over 10 min; and, finally, (iv) isocratic elution at 100% B over 10 min. MS data were collected over 600-2000 m/z in positive ion mode. MS1 data were acquired using the Orbitrap detector (60,000 resolution), and MS/MS in CID mode (ion trap with 30% collision energy). Data analysis was performed using the glycoproteomics toolbox GlycoPAT (Liu et al., 2017), and the Uniprot protein sequence P41148 containing 6 N-X-S/T sites at N62, N107, N217, N445, N481 and N502. Search was performed for a single HexNAc in the case of the EndoH treated samples, and using an N-glycan library with 227 glycans for carbohydrate epitope mapping. Following manual validation of all hits with GlycoPAT ensemble score (ES) >0.4, peak area under the curve was quantified using Xcalibur Quan Browser 3.0 (Thermo).

Cell lines and antibodies for client maturation assay: Grp94-null E4.126 pre-B cells were a gift from Dr. B. Seed (Harvard University, Cambridge, Mass.). Pre-B cells were cultured in RPMI 1640 (Sigma-Aldrich) supplemented with 100 U/ml penicillin and 100 μg/ml streptomycin, 10% FBS (Atlas Biologicals), and 0.055 mM β-me (Life Technologies). Phoenix Eco cells were cultured in DMEM (Sigma-Aldrich) supplemented with 10% FBS and same concentration of penicillin and streptomycin as above. E4.126 and Phoenix Eco cells were cultured at 37° C. with 5% CO2. Grp94 antibody 9G10 was purchased from Enzo Life Sciences and detected both endogenous and overexpressed proteins. The anti-FLAG M2 antibody was purchased from Sigma. Biotin-conjugated anti-mouse CD11a (Clone: M174), CD49d (Clone: R1-2), and TLR2 (Clone: 6C2) antibodies were purchased from Affymetrix and were used to detect endogenous proteins.

Retrovirus production and transduction were carried out as follows. Ecotropic retrovectors (MigR1) containing Grp94 and EGFP were transfected into Phoenix Eco cells using Lipofectamine 2000 (Invitrogen). Two days later, virus-containing medium was collected and added to 2×10⁵ E4.126 cells, along with hexadimethrine bromide (Sigma-Aldrich). Spin infection was achieved by centrifugation at 1900×g for 1.5 hours at 32° C. to facilitate viral transduction.

Flow cytometry analysis was carried out as follows. After retroviral transduction, cell surface expression of the Grp94 client (CD1 1a, CD49d, or TLR2) was evaluated by staining with a client-specific biotin conjugated primary antibody at 4° C. for 30 minutes. Detection was achieved by secondary antibody staining for 30 minutes at 4° C. with a streptavidin-conjugated Allophycocyanin (APC) fluorescent antibody that binds to the biotin-conjugated primary antibody. A biotin-conjugated isotype antibody was used as a negative control to assess background levels. For intracellular staining, which was used to assess the overexpression level of FLAG-tagged Grp94 and the chimeras, cells were fixed in 4% paraformaldehyde at room temperature, and then permeabilized with ice-cold methanol at −20° C. to allow antibody entry. Normal goat serum was used for blocking prior to anti-FLAG antibody or isotope control antibody staining. Antibody staining procedures were similar to surface staining, except all steps were done at room temperature. Antibody or isotype stained cells were acquired on a FACSCalibur (BD Biosciences) and analyzed using the FlowJo software. Using EGFP expression as a positive indicator for retroviral infection, the EGFP-positive cells were analyzed for both surface expression of matured Grp94 clients as well as for the intracellular expression of Grp94.

Size-exclusion chromatography experiments were carried out as follows. Purified Grp94 proteins were adjusted to 5.5 μM in buffer consisting of 40 mM HEPES-KOH (pH 7.4), 150 mM KCl, and 5 mM MgCl2 at a final volume of 1 mL before injection. Each sample was injected onto a Superdex 200 16/60 (GE Healthcare Life Sciences) size-exclusion column equilibrated in 40 mM HEPES-KOH (pH 7.6), 150 mM KCl, and 5 mM MgCl2 at 4° C. at a flow rate of 1.0 mL/min. Absorbance at 280 nm was detected and plotted against the elution time. The void volume was measured using blue dextran (Sigma) as a marker.

Native gel electrophoresis: Samples for Native-PAGE analysis were prepared by pulling-down C-terminally His-tagged Grp94 from total lysates (prepared by detergent lysis in 0.5% CHAPS) using Ni-NTA resin, followed by elution in buffer containing 50 mM Tris-HCl (pH 8.0), 150 mM NaCl, 350 mM Imidazole, and 1 mM β-me. Eluted samples were quantitated by nanodrop. 2 μg of each sample was mixed with 2× Native-PAGE loading buffer and loaded on 4-15% Native gels (Biorad) and resolved for 120 minutes at 150V in Tris-Glycine running buffer under non-reducing conditions. The electrophoresis apparatus was kept cold on ice. The proteins were visualized by coomassie blue staining.

ATP Hydrolysis Assay: ATP hydrolysis rates were measured using the PiPer Phosphate assay kit (Thermo Fisher Scientific) in 96-well fluorescent assay plates (Corning). Assay components were prepared at the recommended stock concentrations in 1× reaction buffer (0.1 M Tris-HCl (pH 7.6)), flash frozen in liquid nitrogen, and stored at −20° C. A stock of −100 mM ATP was prepared in H2O, aliquoted, and frozen at −20° C. Phosphate standards from 0-160 μM were brought up in 2× ATPase buffer (80 mM HEPES-KOH (pH 7.4), 300 mM KCl, and 10 mM MgCl₂). Purified proteins were exchanged into 40 mM HEPES-KOH (pH 7.4), 150 mM KCl, and 5 mM MgCl₂ (1× ATPase buffer), concentrated to 50 and diluted prior to the experiment. The final concentration of protein in each well was 4.5 and the final ATP concentration varied from 0-800 μM. Experimental setup included a 50 μL:50 μL mixture of PiPer reagent (100 μM Amplex Red reagent containing 4 U/mL maltose phosphorylase, 0.4 mM maltose, 2 U/ml glucose oxidase, and 0.4 U/mL horseradish peroxidase) and the ATPase reaction (5 μL ATP stocks and 45 protein (10 μM stock for 4.5 μM final) or 45 μL 1× ATPase buffer for ATP only wells). Plates were incubated at 37° C. for 3 hours and the reactions were quenched on ice.

Fluorescence was measured at 544 nm/590 nm (excitation/emission) on a SpectraMax Gemini XS plate reader (Molecular Devices) with 30 readings per well. Data were corrected using the following equation: enzyme activity=full reaction (all components) −no enzyme control (ATP only background) −no substrate control (enzyme only)+no enzyme/no substrate control (buffer background). ATP hydrolysis experiments are averages of at least three independent measurements. The data were plotted using Prism and fit with the Michaelis-Menten equation.

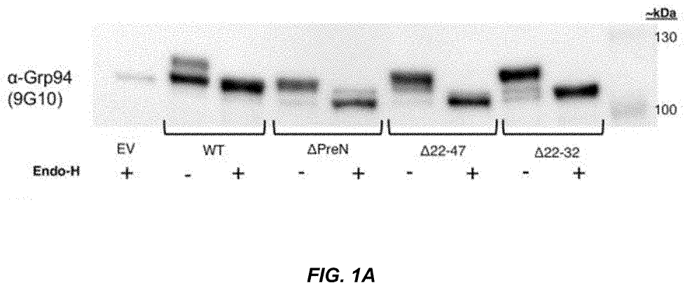

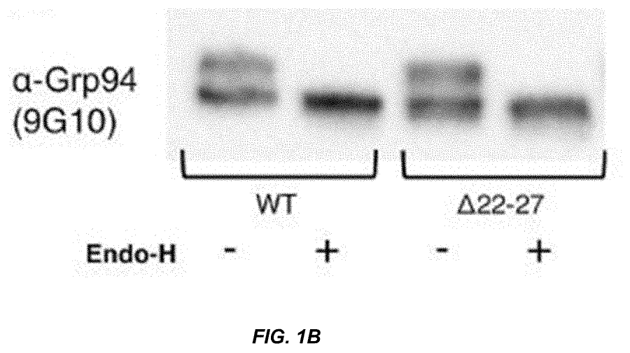

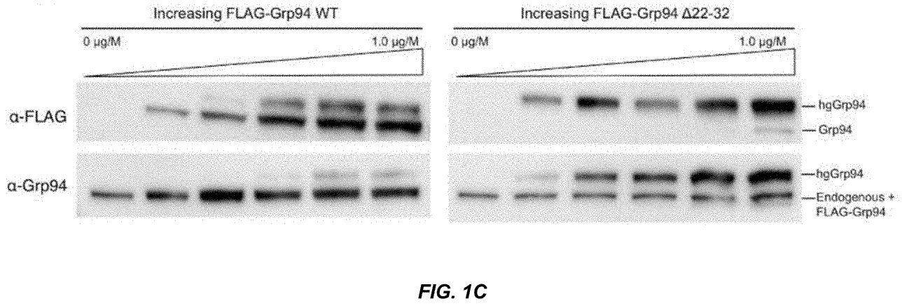

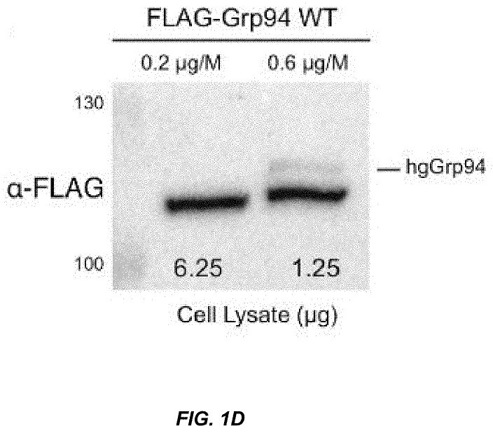

Results of this working example are now discussed with respect to FIGS. 1A-8B.

Previous studies have shown that exogenous overexpression of Grp94 and pharmacological ER stressors induce the formation of a heavily glycosylated Grp94 species termed hyper-glycosylated Grp94 (hgGrp94) (Qu et al., 1994)(Cala, 2000)(Dersh et al., 2014b)(Seidler et al., 2014)(Cherepanova et al., 2019). The hgGrp94 species migrate more slowly than the normal, mono-glycosylated Grp94 (mgGrp94) by SDS-PAGE and western blot, giving rise to at least two distinct species that reflect the number of modified N-linked glycosylation sites. In particular, one study showed that large deletions made from the mature N-terminus of Grp94, including a region roughly corresponding to the domain boundaries of the Pre-N domain, forced the use of additional N-linked glycosylation sites beyond Asn217 (Qu et al., 1994). Based on these observations, we wanted to understand if the Pre-N domain plays a specific role in controlling the glycosylation pattern of Grp94. To do this, we expressed truncated Pre-N domain variants of Grp94 in mammalian cells and evaluated their electrophoretic mobility by western blot.

FIGS. 1A-1D demonstrates that truncation of the Pre-N domain leads to excessive N-linked glycosylation. More specifically, FIG. 1A depicts Western blot analysis of Grp94 hyper-glycosylation. Total lysates were prepared from mammalian cells following transient overexpression for 24 hours of wild type (WT) and Pre-N modified constructs. 7.5 μg of total lysates were run on a 7.5% SDS-PAGE gel, transferred to PVDF, and blotted for Grp94 (9G10). All constructs are sensitive to Endo-H treatment, indicating that the shift in molecular weight is due to high mannose N-linked glycosylation. The molecular weight standards are the PageRuler Plus Pre-stained Ladder (Thermo-Fisher). As shown in FIG. 1B, removal of the first 6 residues beyond the signal peptide does not lead to an increase in hyper-glycosylation compared to WT. Total lysates were prepared as in FIG. 1A. Deletion of residues 22 to 32 leads to a fundamental impairment of N-linked glycosylation (FIG. 1C). Increasing amounts of plasmid DNA (from On per million cells (μg/M) to 1.0 μg/M) were transfected into mammalian cells. The transfection amount increases by 0.2 μg/M per lane. Total lysates were analyzed by western blot using a-FLAG and α-Grp94 antibodies. As further shown in FIG. 1D, hgGrp94^{WT} cannot be detected under low DNA transfection conditions. Five times the amount of the 0.2 μg/M lysate (as determined by densitometry from FIG. 1C) was compared to the 0.6 μg/M lysate.

In agreement with previous studies, the inventors found that high overexpression of wild type Grp94 (Grp94^{WT}) leads to the formation of a hyper-glycosylated species (FIG. 1A). As anticipated, deletion of the Pre-N domain (residues 22-68; Grp94^{ΔPre-N}) results in a marked increase in the amount of hyper-glycosylation compared to Grp94^{WT}. Remarkably, the inventors found that smaller deletions of the Pre-N domain, including Grp94^{Δ22-32} and Grp94Δ²²⁻⁴⁷, also lead to a predominantly hyper-glycosylated species (FIG. 1A). Grp94^{Δ22-32} lacks only the first eleven amino acids of the Pre-N domain, and to see whether hyper-glycosylation arises from altering the junction between the signal peptide (1-21) and the mature protein (22-804), the inventors also examined the glycosylation pattern of Grp94^{Δ22-27}, a 6-residue deletion construct which the inventors previously found retains chaperone function (Huck et al., 2017b). As seen in FIG. 1B, Grp94^{Δ22-27} was hyper-glycosylated only to a similar extent as WT, suggesting that the increase in hyper-glycosylation of larger deletion mutant Grp94^{Δ22-32} does not arise simply due to the deletion of the first few residues following the signal peptide. For each of the constructs tested, the hgGrp94 band was Endo-H sensitive (FIGS. 1A, 1B), indicating that these species contain high mannose N-linked glycans that are characteristic of ER residence. These truncation mutants expressed equally as well as WT.

To better understand the impact of the Pre-N domain on hgGrp94 formation, the inventors transfected increasing amounts of FLAG-tagged Grp94^{WT} and FLAG-tagged Grp94^{Δ22-32} plasmids (0.2-1.0 αg/10⁶ cells) and monitored the presence of hgGrp94 compared to mgGrp94 by both α-FLAG and α-Grp94 (9G10) western blot. As hgGrp94 is of low abundance under endogenous conditions, the inventors predicted that hgGrp94 in a WT expression would increase as the folding demand for this protein rises. Indeed, a concentration-dependent increase in hgGrp94 was observed until saturation of overall expression appeared to have been reached at the 0.8 ag/10⁶ cells transfection level (FIG. 1C). hgGrp94 remained undetectable under low plasmid transfection conditions even when 5-fold more total lysate was loaded on the gel (FIG. 1D), confirming that hyper-glycosylation of WT occurs only after a certain expression threshold had been reached. Strikingly, for Grp94^{Δ22-32}, all newly synthesized protein was hyper-glycosylated even under low expression conditions without the presence of mgGrp94, suggesting that this Pre-N truncation fundamentally disrupts normal N-linked glycosylation of Grp94 (FIG. 1C, top right). For Grp94^{Δ22-32}, only at the highest expression levels (1.0 ag/10⁶ cells) did a lower molecular weight mono- or non-glycosylated species become detectable (see FIG. 1C, right).

The expression of different Pre-N-truncated constructs leads to hyper-glycosylation, but the individual usage and the percent occupancy of each site utilized in hgGrp94 is unknown. To better characterize hgGrp94, purified C-terminally FLAG-tagged Grp94^{Δ22-32} and Grp94ΔPreN recombinant proteins were purified by immunoprecipitation and subjected them to peptide glycosylation analysis by liquid chromatography and tandem mass spectrometry (LC-MS/MS). Prior to tryptic digestion, the samples were treated with Endo-H, which partially de-glycosylates high-mannose N-linked glycans, leaving behind an N-acetlyglucosamine (GlcNAc) moiety that is covalently linked to the asparagine residue. The resulting GlcNAc, if present, results in an increase in peptide mass compared to a non-glycosylated peptide, which can be used to identify which sites are modified and assess the relative frequencies of modified and unmodified peptides.

TABLE 3
Tryptic Peptide Relative Abundance (% Glycosylation).

| | Asn62 | Asn107 | Asn217 | Asn445 | Asn4 | Asn502 |
|---|---|---|---|---|---|---|
| Δ22-32 | 15.9 | 36.2 | 7.68 | 16.0 | n.d. | 24.2 |
| | (56.8%) | (83.0%) | (98.2%) | (78.0%) | | (100%) |
| ΔPre-N | 1.8 | 39.4 | 4.51 | 29.9 | n.d. | 24.6 |
| (22-68) | (7.2%) | (60.8%) | (92.0%) | (83.5) | | (100%) |

Peptide relative abundance quantifies % of peptide (both with and without GlcNAc) detected relative to all potentially glycosylated peptides. Here, label-free quantification of product area under the curve (AUC) is based on the ion current of the precursor mass in the LC-MS/MS chromatogram. All products are validated using MS/MS and LC retention time. n.d. indicates that the peptide could not be detected. Asn62 was likely detected in APre-N preparations due to contaminating endogenous protein. % Glycosylation=100*(AUC of peptide with GlcNAc)/(AUC of peptide with and without GlcNAc).

As summarized in Table 3, the inventors found high levels of glycosylation at all detectable sites (84% average, range 56-100%). (Glycosylation at Asn481 could not be determined, which is likely due to its proximity to multiple tryptic digest sites.) The hgGrp94 populations that result from the small (Δ22-32) and large (ΔPre-N) truncations were nearly identical, with each individual site being more glycosylated than not, suggesting that the glycosylation machinery does not skip specific sites during the process of hyper-glycosylation. Taken together, these results suggest that the Pre-N domain is involved in a specific mechanism to regulate the total amount of Grp94 glycosylation.

The correlation between hyper-glycosylation of Pre-N-truncated constructs and Applicant's previous discovery that the Pre-N domain is required for chaperone function (Huck et al., 2017a) raised the possibility that increased levels of glycosylation directly account for the loss of Grp94 client maturation activity. To determine if N-linked glycosylation at the minor sites is deleterious to chaperone function, the inventors employed an established client maturation assay using a Grp94-null murine pre-B cell line in which Grp94 client integrins (CD11a and CD49d) and TLRs (TLR2) are unable to reach the cell surface in the absence of exogenously expressed Grp94 (Randow and Seed, 2001).

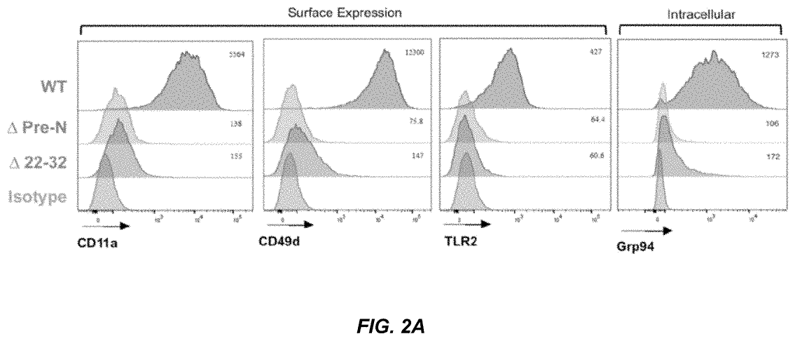

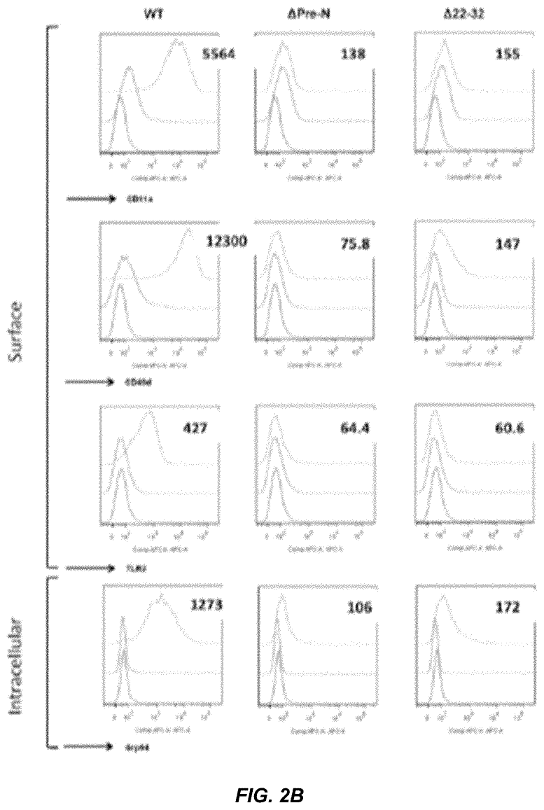

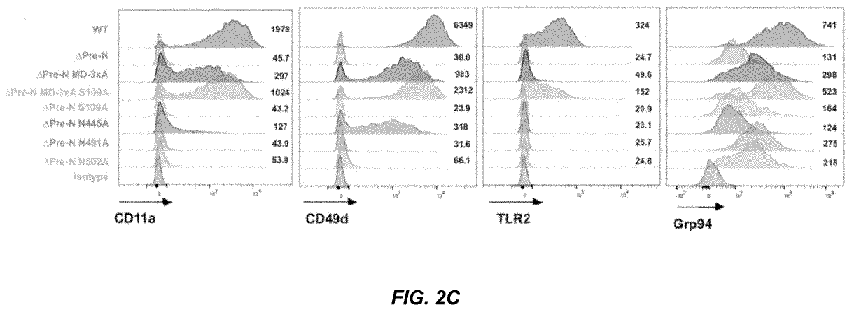

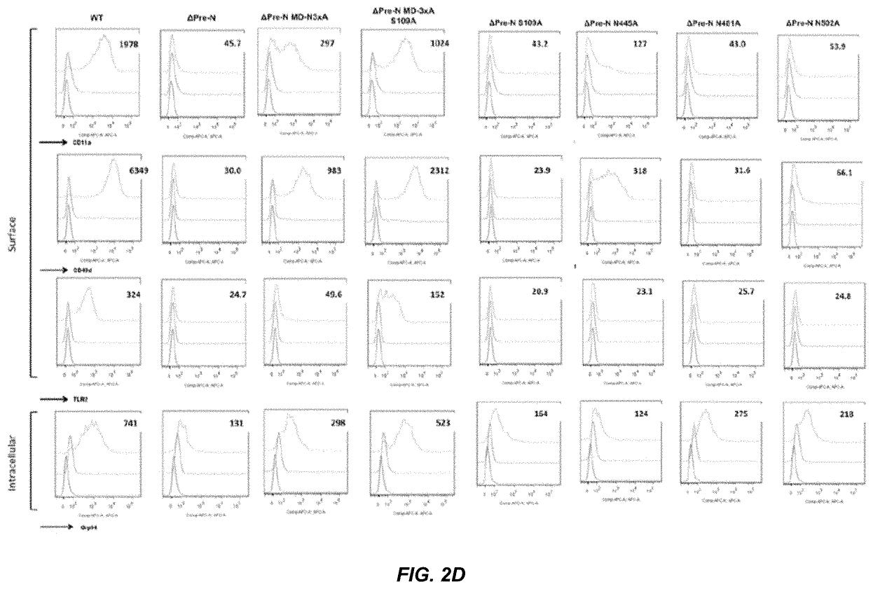

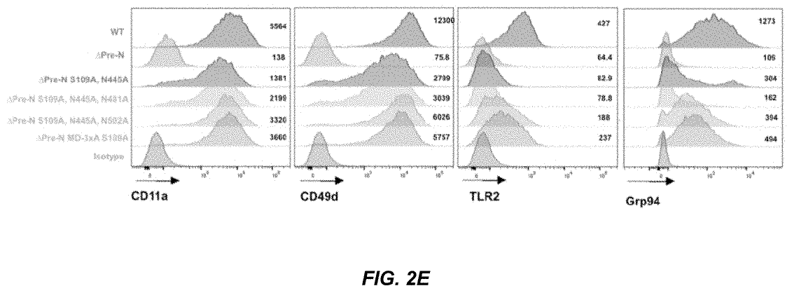

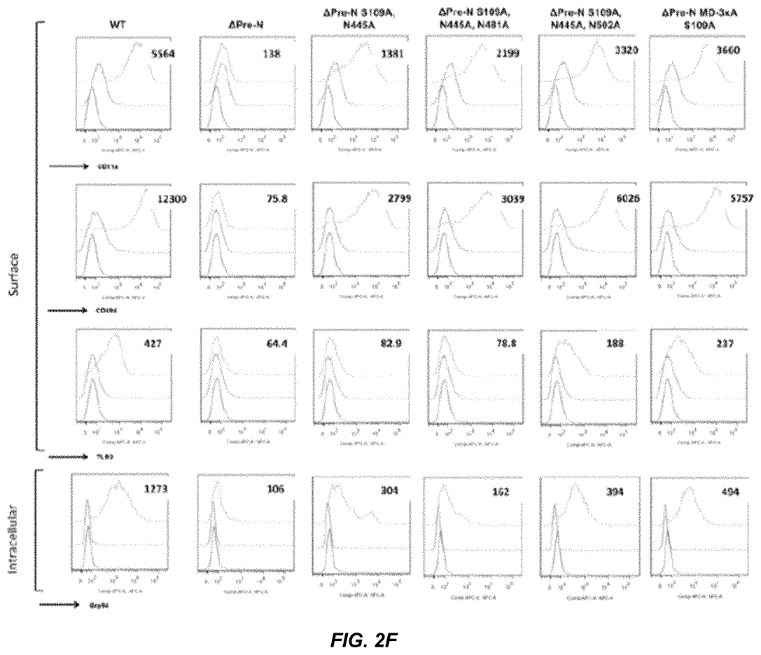

As seen in FIG. 2A, in agreement with Applicant's previous findings, complete deletion of the Pre-N domain eliminates Grp94 chaperone function. In line with Applicant's observation that both Grp94^{ΔPreN} and Grp94^{Δ22-32} were predominantly hyper-glycosylated, Grp94^{Δ22-32} was also completely unable to mature client proteins (FIG. 2A).

When the entire Pre-N domain is deleted and Asn62 is therefore removed, Grp94 retains one major (Asn217) and four minor (Asn107, Asn445, Asn481, and Asn502)N-glycosylation sites. To probe the functional impact of glycosylation at the minor sites, the inventors first mutated the three MD sites Asn445, Asn481, and Asn502 to alanines (MD-N3xA), which prevents their modification. Intriguingly, with the Pre-N domain deleted, the MD-N3xA construct partially restored the surface expression of integrins (14-15%) and TLR2 (10%) relative to WT, suggesting that glycosylation at one or more of these MD glycosylation sites are deleterious to chaperone function (FIG. 2C). When the fourth minor site at Asn107 is also blocked by mutation of Ser109 of the sequon (S109A), chaperone function was restored to almost wild type levels (FIG. 2C). These data suggest that glycosylation at Asn107 plays a pronounced role in reducing the functional population of Grp94.

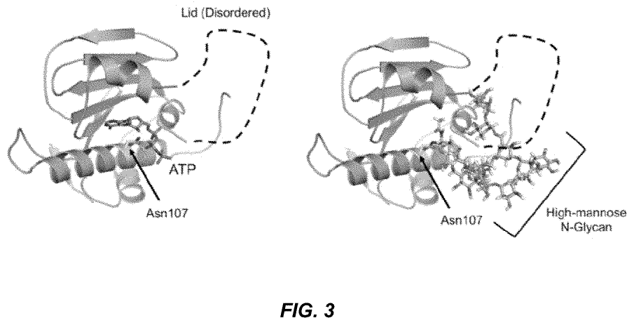

The positive effect of blocking glycosylation at Asn107 is not surprising. Modeling of a high-mannose N-glycan at Asn107 revealed that the addition of a sugar moiety at this residue would adversely affect ATP binding (FIG. 3). Modeling was performed using using the GlyProt server (Bohne-Lang and von der Lieth, 2005) server on the near full-length crystal structure of Grp94 (PDB ID 201U). Shown here is the isolated NTD modeled with a high-mannose N-glycan. Asn107 directly interacts with the phosphates of bound nucleotides, which would likely negatively affect ATP binding.

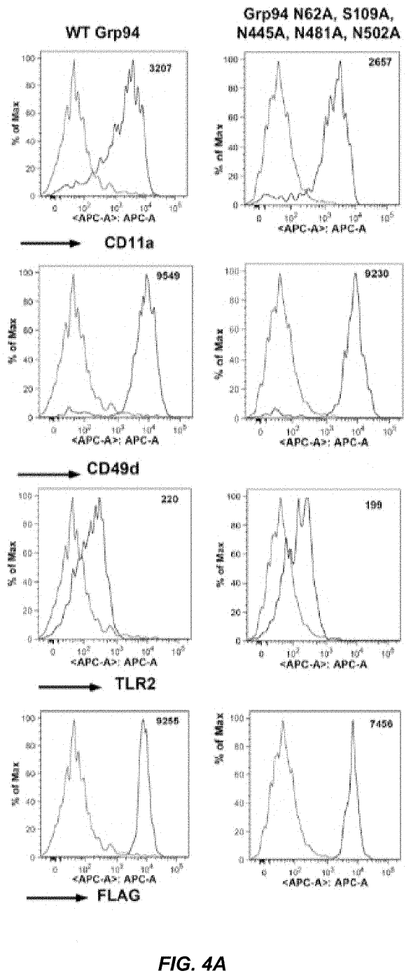

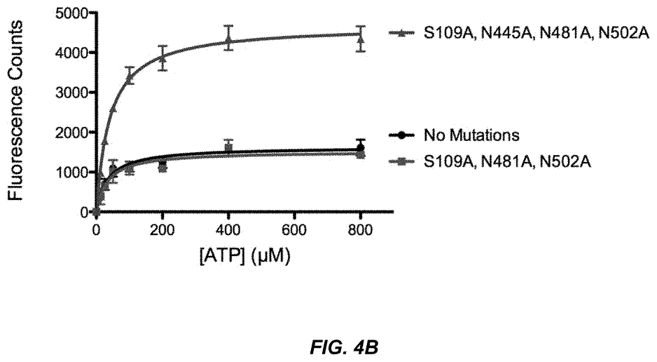

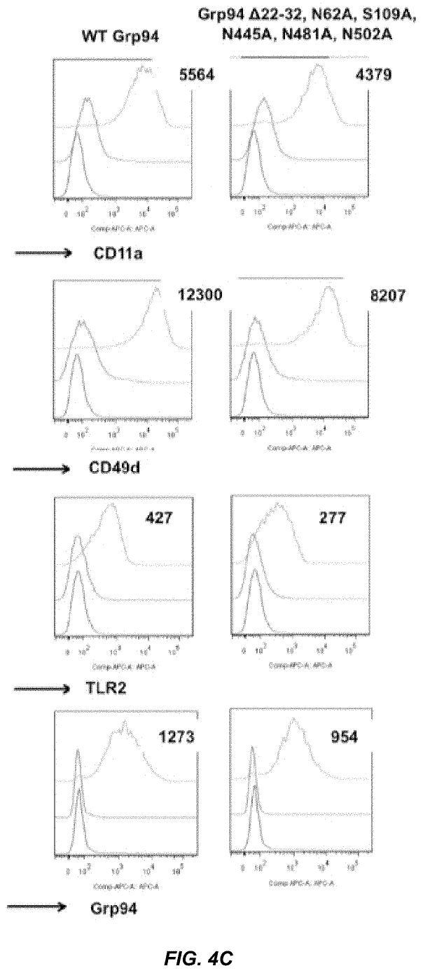

In addition, a large N-glycan tree would be incompatible with normal placement of the open lid conformation of the N-terminal domain and would sterically block the transition of Grp94 to the closed dimer conformation, which is necessary for ATP hydrolysis. Direct mutation of Asn107 to an alanine to block glycosylation is problematic from a mechanistic standpoint since Asn107 directly interacts with the phosphates of bound nucleotides. However, mutation of Ser109 to alanine within the Asn107-Ala108-Ser109 sequon also blocks glycosylation at Asn107 and places the mutation on the opposite face of the helix away from ATP, where it should not affect chaperone function. Mutation of all of the minor sequons does not negatively affect the function of the full-length protein. The right curves were gated on GFP+ cells. The number displayed on the histogram is the Mean Fluorescence Intensity (MFI) of these curves. Intracellular levels of the expressed C-terminally FLAG-tagged Grp94 were determined by staining with FLAG antibody. The left curves were produced by staining with an isotype control antibody (FIG. 4A). Indeed, mutation at Ser109 and all of the minor sequons does not negatively affect the ATPase function of the full-length protein (FIG. 4B).

Similar to the data with Grp94^{ΔPreN}, the blocking mutations at all five minor sites, including Asn62 of the Pre-N domain, restore nearly complete function to Grp94^{Δ22-32} as shown in FIG. 4C. The middle curve was gated on GFP-cells and top curve was gated on GFP+ cells. The number displayed on the histogram is the Mean Fluorescence Intensity (MFI) of the top curve. The bottom curve was produced by staining with an isotype control antibody. The middle curve was gated on GFP+ cells. The number displayed on the histogram is the Mean Fluorescence Intensity (MFI) of the middle curve. Intracellular levels of the expressed C-terminally FLAG-tagged Grp94 were determined by staining with FLAG antibody. The bottom curve was produced by staining with an isotype control antibody.

The inventors next asked if mutation of each minor site alone could restore chaperone function in the context of an otherwise hyperglycosylated Grp94. Surprisingly, although blocking glycosylation at Asn107 restored the majority of the Grp94 chaperoning activity in the context of Grp94ΔPreN, MD-3xA, where the three MD minor sites were also blocked, the S109A mutation alone (Grp94^{ΔPreN,S109A}) was completely nonfunctional. Of the other three minor sites, N445A was the only individual point mutant that was able to mature integrins, albeit at a lower level than WT, but none was capable of maturing TLR2 (FIG. 2C).

Although blockage of individual sites of glycosylation was largely unable to restore Grp94ΔPreN function, blockage of combinations of sites was able to restore activity. The double block mutant S109A/N445A restored a significant fraction of integrin expression, but only slightly restored TLR2 expression. When the inventors combined S109A and N445A with either N481A or N502A, the inventors found that the surface expression of both integrins and TLR2 is enhanced by N502A, but not N481A, suggesting that glycosylation at Asn481 may be functionally benign (FIG. 2E). Collectively, Applicant's results indicate that optimal Grp94 chaperone activity in the absence of the Pre-N domain is achieved when the Asn107, Asn445, and Asn502 glycosylation sequons are mutated to prevent modification at these sites.

For the truncation constructs tested in this assay, the inventors found that the total levels of Grp94 were lower compared to WT when measured using a-Grp94 (9G10) intracellular staining on native protein by flow cytometry (FIGS. 2A-2E). However, the inventors observed an increase in protein levels as glycosylation site mutations were introduced. 9G10 is a conformationally sensitive monoclonal antibody which recognizes an epitope within the charged linker domain of Grp94 (Dersh et al., 2014a; Edwards et al., 1984; Vogen et al., 2002). Low levels of intracellular staining of hgGrp94 are consistent with the notion that hgGrp94 is not natively folded and thus may be subject to impaired antibody binding and/or degradation (Dersh et al., 2014b). Nevertheless, these data identify the Pre-N domain as a protective element of Grp94 function that minimizes the levels of glycosylation at deleterious sites.

FIGS. 2AB, 2D, and 2F show that mutation of minor N-linked glycosylation sites restores chaperone function to Pre-N truncated constructs. Grp94 null pre-B cells were retrovirally transduced with Grp94^{WT}, Grp94^{ΔPre-N}, or Grp94^{Δ22-32} using the MigR1 vector, which co-expresses GFP. The expression of Grp94 clients (CD11a, CD49d, and TLR2) at the cell surface and the intracellular levels of Grp94 were analyzed by flow cytometry using protein-specific antibodies. Representative histograms are displayed. In FIG. 2B, the middle curve was gated on GFP-cells and top curve was gated on GFP+ cells. The number displayed on the histogram is the Mean Fluorescence Intensity (MFI) of the top curve. The bottom curve was produced by staining with an isotype control antibody. Data are representative of at least two independent experiments. In FIGS. 2D and 2F, Grp94 constructs bearing mutations at N-linked glycosylation sequons were transduced and analyzed as in FIG. 2B. MD-3xA refers to the combination of N445A, N481A, and N502A mutants. Otherwise, the specific single, double, or triple mutants are indicated.

The Pre-N Domain is Highly Sequence Specific.

Removal of the first eleven residues of the Pre-N domain is sufficient to favor hyper-glycosylation and, as a result, impede chaperone function, indicating that the presence of this small region is critical.

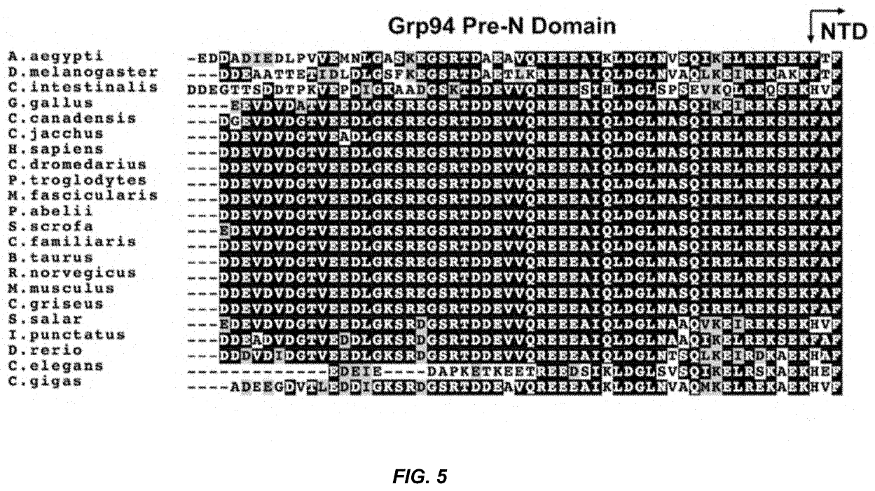

FIG. 5 shows alignment of Pre-N Domain Sequences Across Metazoans. Alignment of the Pre-N domains from various organisms was performed using the mature protein sequence following signal peptide cleavage. The alignment was generated using Clustal Omega (EMBL-EBI) and homology was determined using the BoxShade server (ExPASy). Identical residues are shaded black and homologous residues are shaded gray. Interestingly, the sequence of the entire Pre-N domain is highly conserved throughout metazoans (FIG. 5), suggesting that the role of the Pre-N domain in regulating glycosylation is a required mechanism throughout higher eukaryotic evolution. Moreover, this high degree of conservation suggests that specific residues, and not just the number of amino acids, may be an important contributor to its function.

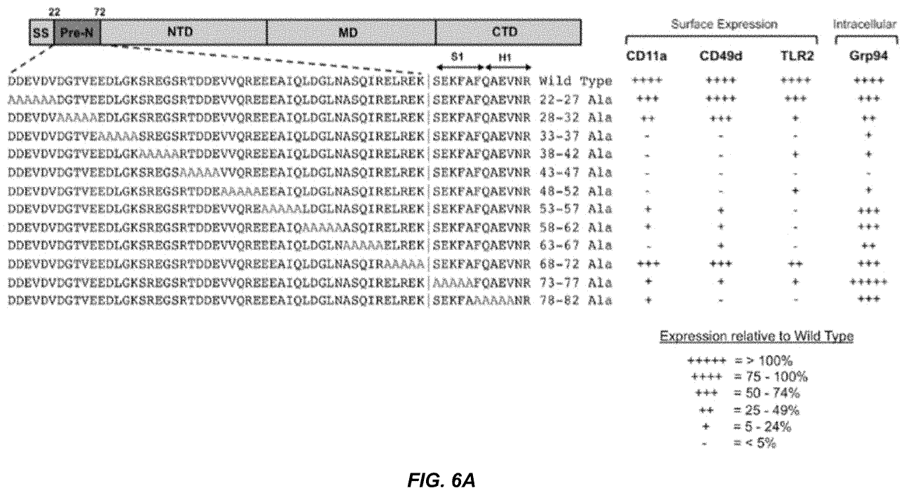

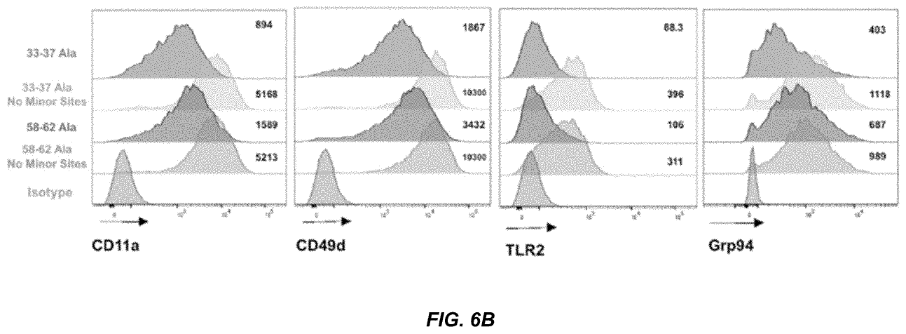

In order to dissect the sequence specificity of the Pre-N Domain, the inventors engineered a set of alanine scan mutants of the approximately fifty (50) Pre-N domain residues. A schematic representation of alanine scan mutants of the Pre-N domain and Strand 1 (51) and into Helix 1 (H1) of the NTD tested by flow cytometry is shown in the left panel of FIG. 6A. Five consecutive residues spanning the length of the Pre-N were mutated to alanine and the resulting constructs were analyzed for chaperone functionality by flow cytometry. As seen in FIG. 6A (right), the Pre-N domain is highly sequence specific; that is, nearly all of the alanine mutants led to diminished client maturation and only those near the N or C termini of the domain (N: 22-27 Ala, 28-32 Ala; C: 68-72 Ala) retained function comparable to WT.

To confirm that these mutants abrogate Grp94 function by increasing deleterious N-linked glycosylation, the inventors mutated the minor N-linked sites and assayed this construct for chaperone function. Grp94 null pre-B cells were retrovirally transduced with Grp94 alanine scan mutants using the MigR1 vector, which co-expresses GFP. The expression of Grp94 clients (CD11a, CD49d, and TLR2) at the cell surface and the intracellular levels of Grp94 were analyzed by flow cytometry using protein-specific antibodies. Representative alanine scan constructs bearing mutations at N-linked glycosylation sequons were transduced and analyzed in the same manner. Representative histograms are displayed (FIG. 6B). As seen in FIG. 6B, mutation of all minor glycosylation sites restores the function of two representative alanine scan mutants to WT levels. Together, these data point to stringent sequence specificity for the Pre-N domain, as relatively small alterations cannot be tolerated without functional impairment.

Hyper-Glycosylated Grp94 Forms Heterogeneous Oligomers that Retain ATPase Activity.

The preceding results show that hyper-glycosylation is deleterious to Grp94 chaperone function, but the mechanism by which the hyper-glycosylated chaperone loses function is unclear. To further investigate the potential causes of the observed loss of function, the inventors purified Grp94^{Δ22-32}, which is fully glycosylated (FIG. 1A), under native conditions and analyzed its oligomeric state by electrophoresis on non-denaturing polyacrylamide gels.

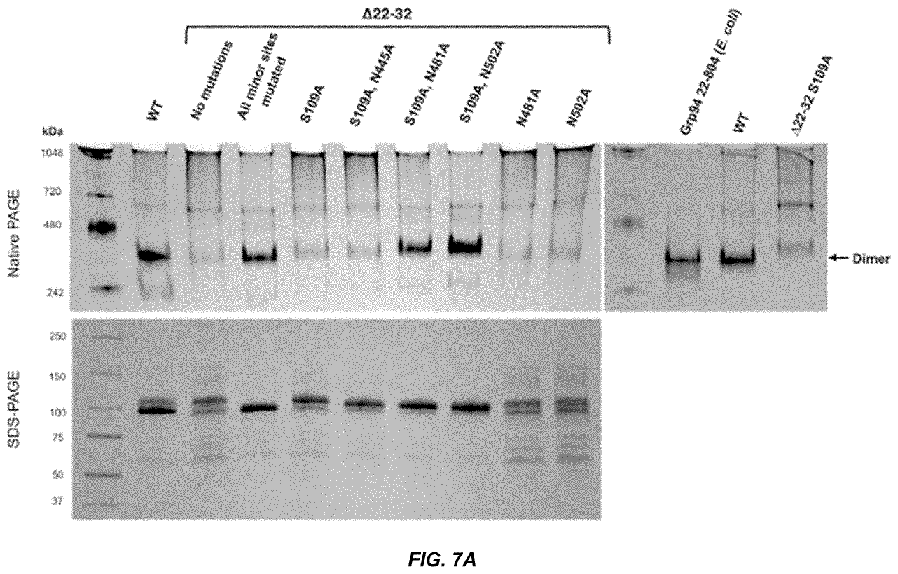

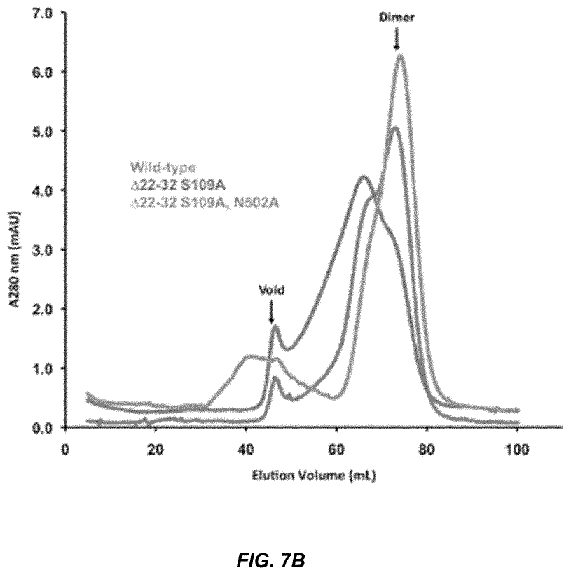

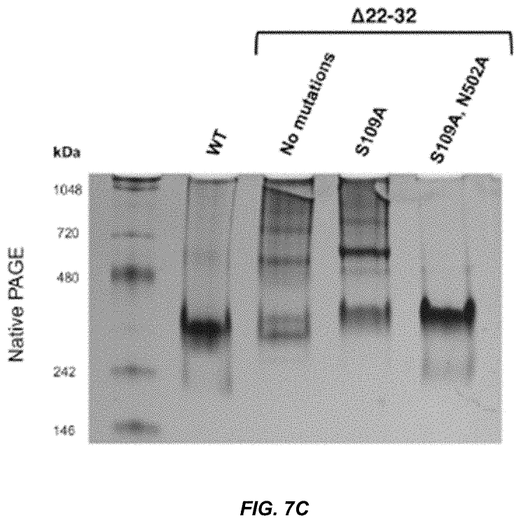

Surprisingly and unexpectedly, compared to WT unglycosylated Grp94, hgGrp94^{Δ22-32} does not migrate as a dimer but instead appears as a heterogeneous high molecular weight species with a significant fraction of the sample failing to enter the gel (FIG. 7A). To test whether glycosylation at one or more of the five minor sites might lead to the formation of the high molecular weight Grp94 oligomers, the inventors made a series of constructs that were mutated at one or more of the minor sites and evaluated the purified mutant proteins for their mobility on Native-PAGE (FIG. 7A, 7C). The inventors found that mutation of the Asn107/Asn502 or Asn107/Asn481 sequon pairs yielded proteins that were predominantly dimeric (FIG. 7A). Mutations to the Asn107, Asn481, and Asn502 sequons alone were not sufficient to restore the protein to a dimer (FIG. 7A).

To further characterize the oligomeric hgGrp94 species, the inventors also analyzed its behavior using size-exclusion chromatography (SEC). As seen in FIG. 7B, hgGrp94^{Δ22-32,S109A}, which runs anomalously by Native-PAGE (FIG. 7A, lane 5), elutes earlier than the dimer as a broad peak. The protein is not excluded from the resin, however, indicating that there is an upper bound to the size of the oligomeric species.

Heterogeneous populations of hgGrp94 are able to hydrolyze ATP. ATPase activity of hgGrp94 derived from mammalian cells or Grp94 derived from E. coli were measured using the fluorescence-based PiPer Phosphate assay (Thermo-Fisher). Control ATPase experiments indicate that S109A, N481A, and N502A mutations do not impact the hydrolysis activity of Grp94 (FIG. 4C). N445A, however, leads to an approximately 4-fold increase in vₘₐₓ. Proteins used in control ATPase experiments were expressed and purified from E. coli.

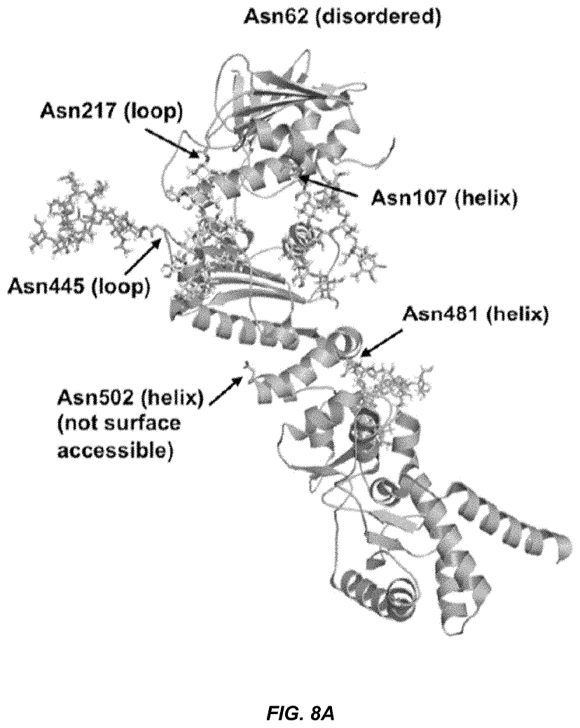

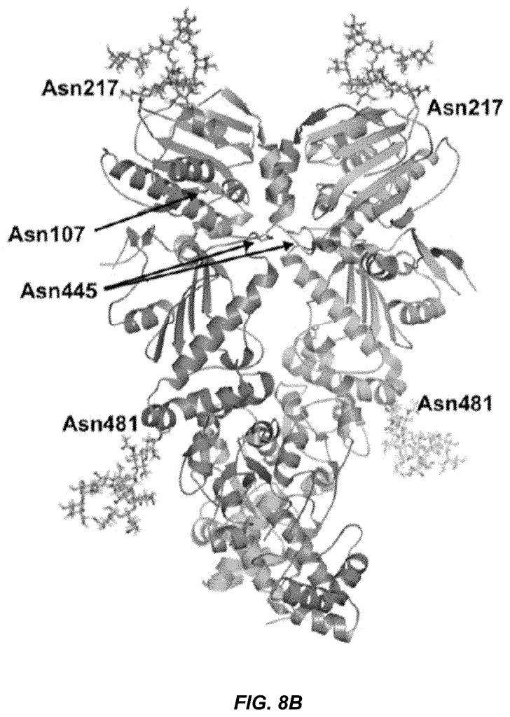

N-glycan modeling analysis of Grp94 in the open state (PDB ID 201U) shows that Asn107, Asn481, and Asn502 are not found on loops, but, rather, at positions that are closely packed into the protein structure, which is uncommon for N-glycan sites. Glycosylation modeling was performed using the GlyProt server on PDB ID 201U. A monomer of the Grp94 dimer is shown for clarity (FIG. 8A). Asn502, in particular, is the only site that is not surface accessible regardless of the side chain rotamer position. This suggests that glycosylation at Asn502 is likely to have a large impact on the Grp94 fold.

Modeling of N-glycans in the closed dimer state of Grp94 (PDB ID 5ULS), on the other hand, reveals that only Asn217 and Asn481 can be modified (FIG. 8B). Asn107 and Asn445 point inward toward the N-terminal dimer interface of the closed dimer conformation, so glycosylation at Asn107 and Asn445 would likely preclude the formation of the closed dimer as they would clash with other structural elements of the N-terminal dimer (FIG. 8B). Since the closed dimer conformation is required for ATP hydrolysis, and because ATPase activity is critical for client maturation, the inventors next asked if purified hgGrp94 was capable of hydrolyzing ATP. Due to their greater purification yield compared to Grp94^{Δ22-32}, the inventors tested proteins bearing mutations at either S109A or S109A/N502A, both of which still which expose the minor sites at residues 62, 445, and 481 for modification. Surprisingly, despite exhibiting oligomeric behavior on Native-PAGE and gel filtration, both proteins were capable of hydrolyzing ATP (FIG. 7C). In fact, Grp94Δ22-32; S109A, which exhibits unusual behavior by Native-PAGE and SEC, has increased ATPase activity when compared to bacterially expressed Grp94. To confirm that the measured ATPase activities are not due to sequon changes, the inventors purified bacterially expressed Grp94 containing S109A and MD-N3xA mutations. Curiously, the mutant construct had 4-fold higher ATPase activity than wild type protein derived from bacteria. Structural inspection of these glycosylation sites in the open state revealed that Asn445 makes stabilizing contacts with the NTD (FIGS. 8A-8B). Reverting 445 back to Asn restored wild type activity, suggesting that the interaction between Asn445 of the MD and the NTD is intrinsically suppressive for Grp94 (FIG. 7D).

Importantly, S109A, N481A, and N502A mutations had no impact on hydrolysis activity, confirming that these mutations did not lead to the observed ATPase activity for HEK293-derived proteins. Together, these data indicate that glycosylation at certain sites may alter the fold of Grp94 and lead to association between Grp94 molecules in an unknown fashion, but does not prevent ATP hydrolysis in the heterogeneous populations purified here.

This disclosure presents the results of an investigation of the role of the Pre-N domain in regulating total levels of Grp94 glycosylation and examined the effects of hyper-glycosylation on chaperone function and ATPase activity. Applicant's results indicate that when normal glycosylation is disrupted by high overexpression or mutations within the Pre-N domain, the chaperone becomes hyper-glycosylated with high glycosylation occupancy at all of the minor sites. Hyper-glycosylation renders the chaperone non-functional for client maturation, but function can be restored by mutating deleterious glycosylation sequons to prevent their modification. As the Pre-N domain is the N-terminal most portion of Grp94, the inventors speculate that this region may direct normal co-translational or post-translational folding and glycosylation during ER translocation. However, a molecular explanation behind how the intact Pre-N domain signals for normal glycosylation and therefore how the artificial lesions within this region tested here disrupt regulation remains unknown so far.

Two of the functionally deleterious sequons uncovered here, Asn107 and Asn445, are strictly conserved throughout hsp90 evolution due to their localization within ATP catalytic regions (Cherepanova et al., 2019). Asn107 is located on helix 2 of the NTD and directly interacts with bound nucleotides as described above, whereas Asn445 is part of the “catalytic loop” in the MD (Prodromou, 2016). Applicant's data show that these minor sites are heavily glycosylated when the Pre-N domain is truncated or absent. All other paralogs possess a shorter Pre-N extension, but retain the conserved sequons equivalent to Asn107 and Asn445 despite not being subject to N-linked glycosylation in their respective subcellular compartments. This observation suggests that one reason the other human paralogs may not functionally complement Grp94 is simply because the equivalent sequons are available for glycosylation. Analogous to Grp94 and the hsp90 family, BiP evolved as a result of gene duplication of an ancestral hsp70 gene. BiP, however, does not possess any N-linked glycosylation sequons nor does it contain a long extension from its N-terminus relative to cytosolic Hsp70. Thus there may exist an evolutionary correlation between the Pre-N domain and the need to self-regulate N-linked glycosylation.

Although Applicant's data indicate that small deletions of the Pre-N domain beyond the signal peptide are sufficient to hyper-glycosylate Grp94, the precise sequence downstream of this region is also highly important for maintaining chaperone activity. The observed specificity suggests that the nearly the entire length of the Pre-N domain, and not just residues 22-32, for example, may participate in specific interactions during co- or post-translational glycosylation of Grp94. As the signal peptide and the Pre-N domain are the first regions to be inserted into the ER, these regions may interact with components of the translocation or N-linked glycosylation machinery. As such, an unknown protein might recognize an extended surface or fold of the Pre-N domain early in translocation, leading to an interaction that ensures normal glycosylation is carried out. In support of this theory, the inventors found that wild type Grp94 is mono-glycosylated until an expression threshold has been reached. A hypothetical partner protein could become saturated at high Grp94 expression levels, which could then shunt a portion of newly synthesized Grp94 toward an alternate glycosylation pathway. Similar to the effect of saturating expression, truncation of the Pre-N domain led to a fundamental impairment of normal glycosylation, i.e. the critical interaction(s) cannot occur, and thus all newly synthesized mutant Grp94 becomes hyper-glycosylated. Alternatively, as pointed out by Qu et al., the usage of certain sites may be dictated by whether or not the specific site in question passes through the membrane co-translationally or post-translationally (Qu et al., 1994). In this way, the Pre-N domain and flanking regions may pause translation allowing the downstream amino acids to be translated and partially folded in the cytosol, which could lead the glycosylation machinery to skip the minor sites. A similar example is found in the N-terminal most portion of the simian rotavirus protein, VP7, but not the closely related bovine VP7, where the signal peptide and residues 64111 delay translocation into the ER (Stirzaker et al., 1990).

In multicellular organisms, OST is a membrane embedded complex consisting of at least eight subunits (Mohorko et al., 2011) (Braunger et al., 2018). Higher eukaryotes possess two paralogs of the catalytic subunit, STT3A and STT3B, with each associating with shared subunits as well as additional unique subunits. A recent study found that deletion of STT3A in cells led to hyper-glycosylation of all five of the minor glycosylation sites of Grp94 (Cherepanova et al., 2019), a phenomenon that appears to phenocopy the effect of mutating the Pre-N domain. Initially, it might seem surprising that genetic deletion a catalytic subunit could lead to an increase in glycosylation levels of Grp94. However, the authors speculate that there is a particular mechanism that normally restricts access of Grp94 to STT3A, which skips the minor sites and normally glycosylates Asn217. In this model, with STT3A deleted, STT3B must glycosylate Grp94 and it presumably does so without skipping any of the minor sites. Undoubtedly, determining if the Pre-N domain interacts specifically with STT3A or other proteins in the OST complex is an important area of future inquiry.

The question persists as to whether the expression of hgGrp94 is a programmed biological mechanism or if it forms simply as an unintended consequence of overexpression left only to be degraded as rapidly as possible. Arguing for functional value in humans is the fact that vertebrates retain six glycosylation sites, yet orthologs in invertebrates such as C. elegans and D. melanogaster have only two or three sites. It remains unclear if certain sites can be selectively modified or if hyper-glycosylation occurs stochastically to indiscriminately shut off activity. Does the biological fate of certain multi- or hyper-glycosylated species differ? Dersh et al. found that, in general, hgGrp94 is degraded at a faster rate than mgGrp94 by an OS-9 mediated, lysosomal-like mechanism (Dersh et al., 2014b). Yet, they found that only ˜50% of hgGrp94 is degraded over a period of 10-24 hours using pulse chase, indicating that this process is not rapid. Applicant's data supports the notion that hgGrp94 might be prone to degradation, as the inventors consistently observed lower intracellular levels of Pre-N truncated Grp94 compared to WT by flow cytometry and also observed that purified hgGrp94 has atypical behavior by Native-PAGE and SEC. Since OS-9 is involved in ERAD, it is unclear why these two proteins associate and get degraded, but yet they do so via an ERAD-independent mechanism.

In summary, the data presented here show that the Pre-N domain, a divergent structural feature within the hsp90 family, controls the total levels of N-linked glycosylation of Grp94. Further, the inventors revealed that this unique mechanism negatively regulates Grp94 activity under conditions of high Grp94 overexpression. Applicant's findings may provide context for cellular conditions in which Grp94 is highly up-regulated, including ER stress and multiple distinct cancers (Lee, 2014). Intriguingly, a recent study found that Grp94 is glycosylated at Asn107 in ovarian cancer cells (Sun and Zhang, 2015), suggesting that a significant portion of the total Grp94 population is non-functional for client maturation. It would be interesting to determine if the non-functional population of Grp94 is beneficial or pathological in the context of disease. In the former scenario, hgGrp94 could hypothetically help maintain ER proteostasis by binding misfolded proteins and facilitating their degradation alongside its own.

Further detail to enable a person of ordinary skill in the art to make and use the techniques described herein is provided in the following references, which are incorporated herein by reference and constitute part of the present disclosure: Huck, J. D., Que, N. L., Hong, F., Li, Z., and Gewirth, D. T. (2017a). Structural and Functional Analysis of Grp94 in the Closed State Reveals an Essential Role for the Pre-N Domain and a Potential Client-Binding Site. Cell Rep 20, 2800-2809; Huck, J. D., Que, N. L., Hong, F., Li, Z., and Gewirth, D. T. (2017b). Structural and Functional Analysis of Grp94 in the Closed State Reveals an Essential Role for the Pre-N Domain and a Potential Client-Binding Site. Cell reports 20, 2800-2809.

1. A recombinant glucose-regulated protein 94 (Grp94) protein comprising:
deletion of seven or more residues from a Pre-N domain of the recombinant Grp94; or
substitution of five or more residues of the Pre-N domain with alanine,
wherein the recombinant Grp94 is glycosylated at a minor glycosylation site.

2. The recombinant Grp94 protein of claim 1, wherein the recombinant Grp94 comprises SEQ ID NO. 2, SEQ ID NO. 3, or SEQ ID NO. 4.

3. The recombinant Grp94 protein of claim 1, wherein the recombinant Grp94 comprises SEQ ID NO. 5, SEQ ID NO. 6, SEQ ID NO. 7, or SEQ ID NO. 8.

4. The recombinant Grp94 protein of claim 1, wherein the minor glycosylation site comprises a glycosylation sequon, wherein the glycosylation sequon is Asn-X-Ser or Asn-X-Thr, and wherein X is any amino acid except proline.

5. The recombinant Grp94 protein of claim 1 further comprising a histidine tag, a flag tag, or a biotin tag.

6. The recombinant Grp94 protein of claim 1, wherein the recombinant Grp94 protein is a fusion protein and further comprises a glycosylated protein.

7. The recombinant Grp94 protein of claim 6, wherein the glycosylated protein is fused to the C-terminus of the Grp94 via a cleavable linker.

8. A method of producing the recombinant Grp94 protein of claim 1 comprising culturing a host cell including a vector which comprises a nucleic acid encoding the recombinant Grp94 protein under conditions permitting the production of the recombinant fusion protein.

9. The method of claim 8, wherein the vector comprises SEQ ID NO. 35, SEQ ID NO. 36, or SEQ ID NO. 34.

10. The method of claim 8, wherein the vector comprises SEQ ID NO. 48, SEQ ID NO. 49, SEQ ID NO. 50, or SEQ ID NO. 51.

11. The method of claim 8 further comprising a step of recovering the recombinant fusion protein.

12. A method of glycosylating a protein comprising:
expressing a recombinant fusion protein in a host cell,
wherein the recombinant fusion protein comprises (i) a wild type glucose-regulated protein 94 (Grp94) or a recombinant Grp94 and (ii) the protein, wherein the protein fused to the C-terminus of the wild type or recombinant Grp94.

13. The method of claim 12, wherein the recombinant fusion protein comprises the recombinant Grp94, and wherein the recombinant Grp94 comprises deletion of seven or more residues from a Pre-N domain of the recombinant Grp94.

14. The method of claim 13, wherein the recombinant Grp94 comprises SEQ ID NO. 2, SEQ ID NO. 3, or SEQ ID NO. 4.

15. The method of claim 12, wherein the recombinant fusion protein comprises the recombinant Grp94, and wherein the recombinant Grp94 comprises substitution of five or more residues of the Pre-N domain with alanine.

16. The method of claim 15, wherein the recombinant Grp94 comprises SEQ ID NO. 5, SEQ ID NO. 6, SEQ ID NO. 7, or SEQ ID NO. 8.

17. The claim 12, wherein the recombinant fusion protein further comprises a histidine tag, a flag tag, or a biotin tag, and wherein the method further comprises isolating the recombinant fusion protein.

18. The method of claim 12, wherein the protein is fused to the C-terminus of the Grp94 via a cleavable linker, and wherein the method further comprises cleaving the protein from the wild type or recombinant Grp94.

19. The method of claim 12, protein wherein the protein comprises a glycosylation sequon, wherein the glycosylation sequon is Asn-X-Ser or Asn-X-Thr, and wherein X is any amino acid except proline.

20. The method of claim 19, further comprising coexpressing oligosaccharyltransferase (OST) complex.


SEQUENCE LISTING (64 sequences)

SEQ ID NO: 1 - protein, 804 aa, Canis familiaris
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 2 - protein, 793 aa, Artificial Sequence
  Grp94_D22-32
MRALWVLGLCCVLLTFGSVRAEDLGKSREGSRTDDEVVQREEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 3 - protein, 778 aa, Artificial Sequence
  Grp94 D22-47
MRALWVLGLCCVLLTFGSVRAVVQREEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 4 - protein, 757 aa, Artificial Sequence
  Grp94_DPreN
MRALWVLGLCCVLLTFGSVRALREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 5 - protein, 804 aa, Artificial Sequence
  Grp94 33-37Ala
MRALWVLGLCCVLLTFGSVRADDEVDVDGTVEAAAAASREGSRTDDEVVQREEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 6 - protein, 804 aa, Artificial Sequence
  Grp94 38-42Ala
MRALWVLGLCCVLLTFGSVRADDEVDVDGTVEEDLGKAAAAARTDDEVVQREEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 7 - protein, 804 aa, Artificial Sequence
  Grp94 43-47Ala
MRALWVLGLCCVLLTFGSVRADDEVDVDGTVEEDLGKSREGSAAAAAVVQREEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 8 - protein, 804 aa, Artificial Sequence
  Grp94 48-52Ala
MRALWVLGLCCVLLTFGSVRADDEVDVDGTVEEDLGKSREGSRTDDEAAAAAEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 9 - protein, 757 aa, Artificial Sequence
  Grp94 DpreN MD3xA
MRALWVLGLCCVLLTFGSVRALREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLAVSRETLQQHKLLKVIRKKLVRKTLDMIKKIADEKYADTFWKEFGTNIKLGVIEDHSARTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 10 - protein, 757 aa, Artificial Sequence
  Grp94 DpreN MD3xA S109A
MRALWVLGLCCVLLTFGSVRALREKSEKFAFQAEVNRMMKLIINSLYKNKEIFLRELISNAADALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLAVSRETLQQHKLLKVIRKKLVRKTLDMIKKIADEKYADTFWKEFGTNIKLGVIEDHSARTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 11 - protein, 757 aa, Artificial Sequence
  Grp94 DpreN S109A
MRALWVLGLCCVLLTFGSVRALREKSEKFAFQAEVNRMMKLIINSLYKNKEIFLRELISNAADALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 12 - protein, 757 aa, Artificial Sequence
  Grp94 DpreN N445A
MRALWVLGLCCVLLTFGSVRALREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLAVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 13 - protein, 757 aa, Artificial Sequence
  Grp94 DpreN N481A
MRALWVLGLCCVLLTFGSVRALREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYADTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 14 - protein, 757 aa, Artificial Sequence
  Grp94_DpreN_N502A
MRALWVLGLCCVLLTFGSVRALREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSARTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 15 - protein, 757 aa, Artificial Sequence
  Grp94_DpreN_S109A_N445A
MRALWVLGLCCVLLTFGSVRALREKSEKFAFQAEVNRMMKLIINSLYKNKEIFLRELISNAADALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLAVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 16 - protein, 757 aa, Artificial Sequence
  Grp94_DpreN_S109A_N445A_N481A
MRALWVLGLCCVLLTFGSVRALREKSEKFAFQAEVNRMMKLIINSLYKNKEIFLRELISNAADALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLAVSRETLQQHKLLKVIRKKLVRKTLDMIKKIADEKYADTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 17 - protein, 757 aa, Artificial Sequence
  Grp94_DpreN_S109A_N445A_N502A
MRALWVLGLCCVLLTFGSVRALREKSEKFAFQAEVNRMMKLIINSLYKNKEIFLRELISNAADALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLAVSRETLQQHKLLKVIRKKLVRKTLDMIKKIADEKYNDTFWKEFGTNIKLGVIEDHSARTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 18 - protein, 793 aa, Artificial Sequence
  Grp94_D22-32_N62A_S109A_N445A_N481A_N502A
MRALWVLGLCCVLLTFGSVRAEDLGKSREGSRTDDEVVQREEEAIQLDGLAASQIRELREKSEKFAFQAEVNRMMKLIINSLYKNKEIFLRELISNAADALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLAVSRETLQQHKLLKVIRKKLVRKTLDMIKKIADEKYADTFWKEFGTNIKLGVIEDHSARTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 19 - protein, 804 aa, Artificial Sequence
  Grp94_28-32Ala
MRALWVLGLCCVLLTFGSVRADDEVDVAAAAAEDLGKSREGSRTDDEVVQREEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 20 - protein, 804 aa, Artificial Sequence
  Grp94_53_57Ala
MRALWVLGLCCVLLTFGSVRADDEVDVDGTVEEDLGKSREGSRTDDEVVQREAAAAALDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 21 - protein, 804 aa, Artificial Sequence
  Grp94_58-62Ala
MRALWVLGLCCVLLTFGSVRADDEVDVDGTVEEDLGKSREGSRTDDEVVQREEEAIQAAAAAASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 22 - protein, 804 aa, Artificial Sequence
  Grp94_63-67Ala
MRALWVLGLCCVLLTFGSVRADDEVDVDGTVEEDLGKSREGSRTDDEVVQREEEAIQLDGLNAAAAAELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 23 - protein, 804 aa, Artificial Sequence
  Grp94_68-72Ala
MRALWVLGLCCVLLTFGSVRADDEVDVDGTVEEDLGKSREGSRTDDEVVQREEEAIQLDGLNASQIRAAAAASEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 24 - protein, 804 aa, Artificial Sequence
  Grp94_73-77Ala
MRALWVLGLCCVLLTFGSVRADDEVDVDGTVEEDLGKSREGSRTDDEVVQREEEAIQLDGLNASQIRELREKAAAAAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 25 - protein, 804 aa, Artificial Sequence
  Grp94_78-82Ala
MRALWVLGLCCVLLTFGSVRADDEVDVDGTVEEDLGKSREGSRTDDEVVQREEEAIQLDGLNASQIRELREKSEKFAAAAAANRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 26 - protein, 810 aa, Artificial Sequence
  Grp94_D22-32_S109A
MRALWVLGLCCVLLTFGSVRAEDLGKSREGSRTDDEVVQREEEAIQLDGLNASQIRELREKSEKFAFQAEVNRMMKLIINSLYKNKEIFLRELISNASDALDKIRLISLTDENAADALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 27 - protein, 810 aa, Artificial Sequence
  Grp94_D22-32_S109A_N445A
MRALWVLGLCCVLLTFGSVRAEDLGKSREGSRTDDEVVQREEEAIQLDGLNASQIRELREKSEKFAFQAEVNRMMKLIINSLYKNKEIFLRELISNASDALDKIRLISLTDENAADALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLAVSRETLQQHKLLKVIRKKLVRKTLDMIKKIADEKYNDTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 28 - protein, 810 aa, Artificial Sequence
  Grp94_D22-32_S109A_N481A
MRALWVLGLCCVLLTFGSVRAEDLGKSREGSRTDDEVVQREEEAIQLDGLNASQIRELREKSEKFAFQAEVNRMMKLIINSLYKNKEIFLRELISNASDALDKIRLISLTDENAADALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYADTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 29 - protein, 810 aa, Artificial Sequence
  Grp94_D22-32_S109A_N502A
MRALWVLGLCCVLLTFGSVRAEDLGKSREGSRTDDEVVQREEEAIQLDGLNASQIRELREKSEKFAFQAEVNRMMKLIINSLYKNKEIFLRELISNASDALDKIRLISLTDENAADALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSARTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 30 - protein, 810 aa, Artificial Sequence
  Grp94_D22-32_N481A
MRALWVLGLCCVLLTFGSVRAEDLGKSREGSRTDDEVVQREEEAIQLDGLNASQIRELREKSEKFAFQAEVNRMMKLIINSLYKNKEIFLRELISNASDALDKIRLISLTDENASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYADTFWKEFGTNIKLGVIEDHSNRTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 31 - protein, 810 aa, Artificial Sequence
  Grp94_D22-32_N502A
MRALWVLGLCCVLLTFGSVRAEDLGKSREGSRTDDEVVQREEEAIQLDGLNASQIRELREKSEKFAFQAEVNRMMKLIINSLYKNKEIFLRELISNASDALDKIRLISLTDENASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEDSDDEAAVEEEEEEKKPKTKKVEKTVWDWELMNDIKPIWQRPSKEVEDDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSARTRLAKLLRFQSSHHPSDITSLDQYVERMKEKQDKIYFMAGSSRKEAESSPFVERLLKKGYEVIYLTEPVDEYCIQALPEFDGKRFQNVAKEGVKFDESEKTKESREAIEKEFEPLLNWMKDKALKDKIEKAVVSQRLTESPCALVASQYGWSGNMERIMKAQAYQTGKDISTNYYASQKKTFEINPRHPLIKDMLRRVKEDEDDKTVSDLAVVLFETATLRSGYLLPDTKAYGDRIERMLRLSLNIDPDAKVEEEPEEEPEETTEDTTEDTEQDDEEEMDAGTDDEEQETVKKSTAEKDEL

SEQ ID NO: 32 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 33 - DNA, 2415 bp, Canis familiaris
atgagggccctgtgggtgctgggcctctgctgcgtcctgctgaccttcgggtcagtccgagctgacgatgaagtcgatgtggatggtacagtggaagaggatctgggtaaaagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 34 - DNA, 2274 bp, Artificial Sequence
  Grp94_DPreN
atgagggccctgtgggtgctgggcctctgctgcgtcctgctgaccttcgggtcagtccgagctcttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 35 - DNA, 2382 bp, Artificial Sequence
  Grp94_D22-32
atgagggccctgtgggtgctgggcctctgctgcgtcctgctgaccttcgggtcagtccgagctgaggatctgggtaaaagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 36 - DNA, 2337 bp, Artificial Sequence
  Grp94_D22-47
atgagggccctgtgggtgctgggcctctgctgcgtcctgctgaccttcgggtcagtccgagctgtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 37 - DNA, 2274 bp, Artificial Sequence
  Grp94_DPreN_MD3xA
atgagggccctgtgggtgctgggcctctgctgcgtcctgctgaccttcgggtcagtccgagctcttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttggctgtttcccgggaaactcttcagcaacataaactgcttaaggtgattagaaagaagcttgtccgtaaaactctggacatgatcaagaagattgctgatgagaagtacgctgatactttttggaaagaatttggtaccaacatcaagcttggtgtaattgaagaccactcagctcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 38 - DNA, 2274 bp, Artificial Sequence
  Grp94_DpreN_MD3xA_S109A
atgagggccctgtgggtgctgggcctctgctgcgtcctgctgaccttcgggtcagtccgagctcttagagaaaaatcagaaaaatttgccttccaagctgaagtgaatagaatgatgaaacttatcatcaattcattgtataaaaataaagagattttcttgagagaactgatttcaaatgctgctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttggctgtttcccgggaaactcttcagcaacataaactgcttaaggtgattagaaagaagcttgtccgtaaaactctggacatgatcaagaagattgctgatgagaagtacgctgatactttttggaaagaatttggtaccaacatcaagcttggtgtaattgaagaccactcagctcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 39 - DNA, 2274 bp, Artificial Sequence
  Grp94_DpreN_S109A
atgagggccctgtgggtgctgggcctctgctgcgtcctgctgaccttcgggtcagtccgagctcttagagaaaaatcagaaaaatttgccttccaagctgaagtgaatagaatgatgaaacttatcatcaattcattgtataaaaataaagagattttcttgagagaactgatttcaaatgctgctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 40 - DNA, 2274 bp, Artificial Sequence
  Grp94_DpreN_N445A
atgagggccctgtgggtgctgggcctctgctgcgtcctgctgaccttcgggtcagtccgagctcttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttggctgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 41 - DNA, 2274 bp, Artificial Sequence
  Grp94_DpreN_N481A
atgagggccctgtgggtgctgggcctctgctgcgtcctgctgaccttcgggtcagtccgagctcttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacgctgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 42 - DNA, 2274 bp, Artificial Sequence
  Grp94_DpreN_N502A
atgagggccctgtgggtgctgggcctctgctgcgtcctgctgaccttcgggtcagtccgagctcttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcagctcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 43 - DNA, 2274 bp, Artificial Sequence
  Grp94_DpreN_S109A_N445A
atgagggccctgtgggtgctgggcctctgctgcgtcctgctgaccttcgggtcagtccgagctcttagagaaaaatcagaaaaatttgccttccaagctgaagtgaatagaatgatgaaacttatcatcaattcattgtataaaaataaagagattttcttgagagaactgatttcaaatgctgctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttggctgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 44 - DNA, 2274 bp, Artificial Sequence
  Grp94_DpreN_S109A_N445A_N481A
atgagggccctgtgggtgctgggcctctgctgcgtcctgctgaccttcgggtcagtccgagctcttagagaaaaatcagaaaaatttgccttccaagctgaagtgaatagaatgatgaaacttatcatcaattcattgtataaaaataaagagattttcttgagagaactgatttcaaatgctgctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttggctgtttcccgggaaactcttcagcaacataaactgcttaaggtgattagaaagaagcttgtccgtaaaactctggacatgatcaagaagattgctgatgagaagtacgctgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 45 - DNA, 2274 bp, Artificial Sequence
  Grp94_DpreN_S109A_N445A_N502A
atgagggccctgtgggtgctgggcctctgctgcgtcctgctgaccttcgggtcagtccgagctcttagagaaaaatcagaaaaatttgccttccaagctgaagtgaatagaatgatgaaacttatcatcaattcattgtataaaaataaagagattttcttgagagaactgatttcaaatgctgctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttggctgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcagctcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 46 - DNA, 2382 bp, Artificial Sequence
  Grp94_D22-32_N62A_S109A_N445A_N481A_N502A
atgagggccctgtgggtgctgggcctctgctgcgtcctgctgaccttcgggtcagtccgagctgaggatctgggtaaaagtagagaaggctccaggacagatgatgaagtagtgcagagagaggaagaagctattcagttggatggattagctgcatcccaaataagagaacttagagaaaaatcagaaaaatttgccttccaagctgaagtgaatagaatgatgaaacttatcatcaattcattgtataaaaataaagagattttcttgagagaactgatttcaaatgctgctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttggctgtttcccgggaaactcttcagcaacataaactgcttaaggtgattagaaagaagcttgtccgtaaaactctggacatgatcaagaagattgctgatgagaagtacgctgatactttttggaaagaatttggtaccaacatcaagcttggtgtaattgaagaccactcagctcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 47 - DNA, 2415 bp, Artificial Sequence
  Grp94_28-32Ala
atgagggccctgtgggtgctgggcctctgctgcgtcctgctgaccttcgggtcagtccgagctgacgatgaagtcgatgtggcagcggcagcggcagaggatctgggtaaaagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 48 - DNA, 2415 bp, Artificial Sequence
  Grp94_33-37Ala
atgagggccctgtgggtgctgggcctctgctgcgtcctgctgaccttcgggtcagtccgagctgacgatgaagtcgatgtggatggtacagtggaagcggcagcggcagcgagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 49 - DNA, 2415 bp, Artificial Sequence
  Grp94_38-42Ala
atgagggccctgtgggtgctgggcctctgctgcgtcctgctgaccttcgggtcagtccgagctgacgatgaagtcgatgtggatggtacagtggaagaggatctgggtaaagcggcagcggcagcgaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 50 - DNA, 2415 bp, Artificial Sequence
  Grp94_43-47Ala
atgagggccctgtgggtgctgggcctctgctgcgtcctgctgaccttcgggtcagtccgagctgacgatgaagtcgatgtggatggtacagtggaagaggatctgggtaaaagtagagaaggctccgcggcagcggcagcggtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 51 - DNA, 2415 bp, Artificial Sequence
  Grp94_48-52Ala
atgagggccctgtgggtgctgggcctctgctgcgtcctgctgaccttcgggtcagtccgagctgacgatgaagtcgatgtggatggtacagtggaagaggatctgggtaaaagtagagaaggctccaggacagatgatgaagcggcagcggcagcggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 52 - DNA, 2415 bp, Artificial Sequence
  Grp94_53_57Ala
atgagggccctgtgggtgctgggcctctgctgcgtcctgctgaccttcgggtcagtccgagctgacgatgaagtcgatgtggatggtacagtggaagaggatctgggtaaaagtagagaaggctccaggacagatgatgaagtagtgcagagagaggcggcagcggcagcgttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 53 - DNA, 2415 bp, Artificial Sequence
  Grp94_58-62Ala
atgagggccctgtgggtgctgggcctctgctgcgtcctgctgaccttcgggtcagtccgagctgacgatgaagtcgatgtggatggtacagtggaagaggatctgggtaaaagtagagaaggctccaggacagatgatgaagtagtgcagagagaggaagaagctattcaggcggcagcggcagcggcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 54 - DNA, 2415 bp, Artificial Sequence
  Grp94_63-67Ala
atgagggccctgtgggtgctgggcctctgctgcgtcctgctgaccttcgggtcagtccgagctgacgatgaagtcgatgtggatggtacagtggaagaggatctgggtaaaagtagagaaggctccaggacagatgatgaagtagtgcagagagaggaagaagctattcagttggatggattaaatgcggcagcggcagcggaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 55 - DNA, 2415 bp, Artificial Sequence
  Grp94_68-72Ala
atgagggccctgtgggtgctgggcctctgctgcgtcctgctgaccttcgggtcagtccgagctgacgatgaagtcgatgtggatggtacagtggaagaggatctgggtaaaagtagagaaggctccaggacagatgatgaagtagtgcagagagaggaagaagctattcagttggatggattaaatgcatcccaaataagagcggcagcggcagcgtcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 56 - DNA, 2415 bp, Artificial Sequence
  Grp94_73-77Ala
atgagggccctgtgggtgctgggcctctgctgcgtcctgctgaccttcgggtcagtccgagctgacgatgaagtcgatgtggatggtacagtggaagaggatctgggtaaaagtagagaaggctccaggacagatgatgaagtagtgcagagagaggaagaagctattcagttggatggattaaatgcatcccaaataagagaacttagagaaaaagcggcagcggcagcgttccaagctgaagtgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 57 - DNA, 2415 bp, Artificial Sequence
  Grp94_78-82Ala
atgagggccctgtgggtgctgggcctctgctgcgtcctgctgaccttcgggtcagtccgagctgacgatgaagtcgatgtggatggtacagtggaagaggatctgggtaaaagtagagaaggctccaggacagatgatgaagtagtgcagagagaggaagaagctattcagttggatggattaaatgcatcccaaataagagaacttagagaaaaatcagaaaaatttgccgcggcagcggcagcgaatagaatgatgaaacttatcatcaattcattgtataaaaataaagagattttcttgagagaactgatttcaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 58 - DNA, 2433 bp, Artificial Sequence
  Grp94_D22-32_S109A
atgagggccctgtgggtgctgggcctctgctgcgtcctgctgaccttcgggtcagtccgagctgaggatctgggtaaaagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctgctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 59 - DNA, 2433 bp, Artificial Sequence
  Grp94_D22-32_S109A_N445A
atgagggccctgtgggtgctgggcctctgctgcgtcctgctgaccttcgggtcagtccgagctgaggatctgggtaaaagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctgctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttggctgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 60 - DNA, 2433 bp, Artificial Sequence
  Grp94_D22-32_S109A_N481A
atgagggccctgtgggtgctgggcctctgctgcgtcctgctgaccttcgggtcagtccgagctgaggatctgggtaaaagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctgctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacgctgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 61 - DNA, 2433 bp, Artificial Sequence
  Grp94_D22-32_S109A_N502A
atgagggccctgtgggtgctgggcctctgctgcgtcctgctgaccttcgggtcagtccgagctgaggatctgggtaaaagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgctgctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcagctcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 62 - DNA, 2433 bp, Artificial Sequence
  Grp94_D22-32_N481A
atgagggccctgtgggtgctgggcctctgctgcgtcctgctgaccttcgggtcagtccgagctgaggatctgggtaaaagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacgctgatactttttggaaagaatttggtaccaacatcaagcttggtgtaattgaagaccactcaaatcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 63 - DNA, 2433 bp, Artificial Sequence
  Grp94_D22-32_N502A
atgagggccctgtgggtgctgggcctctgctgcgtcctgctgaccttcgggtcagtccgagctgaggatctgggtaaaagtagagaaggctccaggacagatgatgaagtagtgcagagagaggaagaagctattcagttggatggattaaatgcatcccaaataagagaacttagagaaaaatcagaaaaatttgccttccaagctgaagtgaatagaatgatgaaacttatcatcaattcattgtataaaaataaagagattttcttgagagaactgatttcaaatgcttctgatgccttagataagataaggttaatatcactgactgatgaaaatgcttctgatgccttagataagataaggttaatatcactgactgatgaaaatgctcttgctggaaatgaggaactaactgtcaaaattaagtgtgacaaggagaagaatctgctacatgtcacagacactggtgtgggaatgacccgggaagagttggttaaaaaccttggtaccatagccaaatctggaacaagcgagtttttaaacaaaatgactgaggcacaagaggatggccagtcaacttctgaactgattgggcagtttggtgtcggtttctattctgccttccttgtcgcagataaggttattgtcacatcaaaacacaacaacgatacccagcatatctgggaatctgactccaatgagttctctgtaattgctgacccacgagggaacaccctcggacggggaacaacaattacacttgttttaaaagaagaagcatctgattaccttgaattggacacaattaaaaatctcgtcaagaaatattcacagtttataaacttccctatttatgtgtggagcagcaagactgaaactgttgaggagcccatggaagaagaagaagcagcaaaagaagaaaaagaagattctgatgatgaagctgcagtggaagaagaagaggaggaaaaaaaaccaaaaaccaaaaaagttgagaaaactgtctgggattgggagcttatgaatgacatcaaaccaatatggcagagaccatcaaaagaagtagaagatgacgaatacaaagctttctacaaatcattttcaaaggaaagtgatgaccccatggcttatatccactttactgctgaaggggaagtcaccttcaaatcaattttatttgtacctacatctgctccacgtggtctgtttgatgaatatggatctaagaagagtgattacattaagctttacgtgcgcagagtattcatcacagatgacttccatgatatgatgcccaagtaccttaactttgtcaagggtgttgtggactcagatgatctccccttgaatgtttcccgggaaactcttcagcaacataaactgcttaaggtgattagaaagaagcttgtccgtaaaactctggacatgatcaagaagattgctgatgagaagtacaatgatactttttggaaagaatttggtaccaacatcaagcttggtgtaattgaagaccactcagctcgaacacgtcttgctaaacttcttagattccagtcatctcatcatccaagtgacataaccagtctagaccaatacgtggaaagaatgaaggagaagcaagacaaaatctacttcatggctgggtctagcagaaaagaggctgaatcttctccatttgttgagcgacttctgaaaaagggctatgaagtgatttatctcaccgaacctgtggacgaatactgcattcaggctcttcctgagtttgatgggaaaaggttccagaatgttgccaaagaaggtgtgaaatttgatgaaagtgagaaaacaaaggagagtcgtgaagcgattgagaaagaatttgagcctctgctcaactggatgaaagataaagctctcaaggacaagattgaaaaggccgtggtatctcagcgtctgacagagtctccgtgtgctctggtggccagccagtatggatggtctggcaacatggagagaatcatgaaagctcaagcataccagacgggcaaagacatctctacaaattactatgccagccaaaagaaaacatttgaaattaatcccagacatcccctgatcaaagacatgctgcgacgagttaaggaagatgaagatgacaaaacggtatcggatcttgctgtggttttgtttgagacagcaacgctgagatcaggctatctgctaccagacactaaagcatatggagatcgaatagaaagaatgcttcgcctcagtttaaacattgaccctgatgcaaaggtggaagaagaaccagaagaagaacccgaagagacaaccgaggacaccacagaagacacagagcaggacgatgaagaagaaatggatgcaggaacagacgacgaagaacaagaaacagtaaagaaatctacagctgaaaaagatgaattataa

SEQ ID NO: 64 - DNA, 2412 bp, Homo sapiens
atgagggccctgtgggtgctgggcctctgctgcgtcctgctgaccttcgggtcggtcagagctgacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaaaagatgaattgtaa